(19) United States
(12) Patent Application Publication
(10) Pub. No.: US 2010/0152047 A1
(43) Pub. Date: Jun. 17, 2010

(54) Piperazine Compounds Whith a Herbicidal Action

(75) Inventors: Eike Hupe, Mannheim (DE); Thomas Seitz, Viernheim (DE); Matthias Witschel, Bad Duerkheim (DE); Dschun Song, Mannheim (DE); William Karl Moberg, Hassloch (DE); Liliana Parra Rapado, Offenburg (DE); Frank Stelzer, Mannheim (DE); Andrea Vescovi, Mannheim (DE); Trevor William Newton, Neustadt (DE); Robert Reinhard, Limburgerhof (DE); Bernd Sievernich, Hassloch (DE); Klaus Grossmann, Neuhofen (DE); Thomas Ehrhardt, Speyer (DE); Michael Rack, Eppelheim (DE); Elmar Kibler, Hassloch (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/663,784

(22) PCT Filed: Jun. 11, 2008
(86) PCT No.: PCT/EP08/57329

§ 371 Date: Dec. 9, 2009

(30) Foreign Application Priority Data
Jun. 12, 2007 (EP) 07110124.0

(51) Int. Cl. A01N 43/58; C07D 403/10; C07D 401/10; C07D 241/02; C07D 413/10; A01N 43/54; A01N 43/60; A01P 13/00

(52) U.S. Cl. 504/236; 544/295; 544/360; 544/366; 544/370; 544/357; 544/238; 544/369; 504/239; 504/235

(57) ABSTRACT

The present invention relates to piperazine compounds of the general formula I defined below and to their use as herbicides. Moreover, the invention relates to compositions for crop protection and to a method for controlling unwanted vegetation.

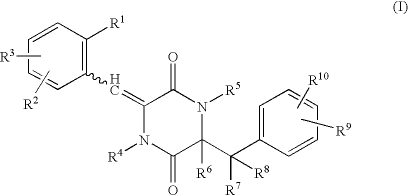

In formula I, the variables have the following meanings:
R¹ is selected from the group consisting of halogen, cyano, nitro, Z—C(═O)—R¹¹, phenyl and a 5- or 6-membered heterocyclic radical which has 1, 2, 3 or 4 heteroatoms selected from the group consisting of O, N and S as ring atoms, where phenyl and the heterocyclic radical are unsubstituted or may have 1, 2, 3 or 4 substituents R^{1a};
   Z is a covalent bond or a CH₂ group;
   R¹¹ is hydrogen, C₁-C₆-alkyl, C₃-C₆-cycloalkyl, C₂-C₆-alkenyl, C₅-C₆-cycloalkenyl, C₂-C₆-alkynyl and the like;
R² is hydrogen, halogen, nitro, cyano, C₁-C₄-alkyl, C₁-C₄-haloalkyl, C₂-C₄-alkenyl, C₁-C₄-alkoxy, C₁-C₄-haloalkoxy, benzyl or a group S(O)ₙR²¹ in which R²¹ is C₁-C₄-alkyl or C₁-C₄-haloalkyl and n is 0, 1 or 2;
R³ is hydrogen or halogen;
R⁴ is C₁-C₄-alkyl, C₃-C₄-alkenyl or C₃-C₄-alkynyl;
   R⁵ is hydrogen, C₁-C₄-alkyl, C₃-C₄-alkenyl, C₃-C₄-alkynyl or a group C(═O)R⁵¹ in which R⁵¹ is hydrogen, C₁-C₄-alkyl, C₁-C₄-haloalkyl, C₁-C₄-alkoxy or C₁-C₄-haloalkoxy;
R⁶ is C₁-C₄-alkyl, C₁-C₄-hydroxyalkyl or C₁-C₄-haloalkyl;
R⁷, R⁸ independently of one another are hydrogen, OH, C₁-C₄-alkoxy, C₁-C₄-haloalkyloxy, C₁-C₄-alkyl or C₁-C₄-haloalkyl; and
R⁹, R¹⁰ independently of one another are selected from the group consisting of hydrogen, halogen, CN, NO₂, C₁-C₄-alkyl, C₁-C₄-haloalkyl, C₂-C₄-alkenyl, C₁-C₄-alkoxy and C₁-C₄-haloalkoxy.

The present invention relates to piperazine compounds of the general formula I defined below and to their use as herbicides. Moreover, the invention relates to compositions for crop protection and to a method for controlling unwanted vegetation.

The thaxtomins A and B (King R. R. et al., J. Agric. Food Chem. (1992) 40, 834-837), which are produced by the plant pathogen S. scabies, are natural products having a central piperazine-2,5-dione ring which carries a 4-nitroindol-3-ylmethyl radical in the 3-position and an optionally OH-substituted benzyl radical in the 2-position. Because of their plant-damaging activity, this class of compounds was also examined for a possible use as herbicides (King R. R. et al., J. Agric. Food Chem. (2001) 49, 2298-2301).

In the context of synthetic investigations into the preparation of thaxtomin A and B, J. Gelin et al., J. Org. Chem. 58, 1993, pp. 3473-3475, and J. Moyroud et al., Tetrahedron 52, 1996, pp. 8525-8543 describe dehydrothaxtomin derivatives. Described are, inter alia, compounds of the formula

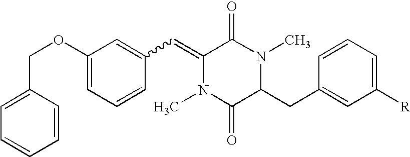

in which R is hydrogen or NO₂.

N. Saito et al., J. Chem. Soc. Perkin Trans 1997, pp. 53-69 describe, inter alia, compounds of the formula below

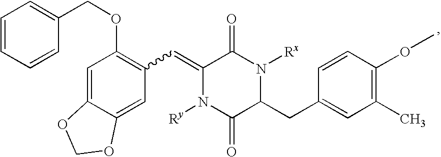

in which R^{y} is hydrogen or benzyl and R^{x} is hydrogen, acetyl or isopropyloxycarbonyl as precursors for the preparation of ecteinascidins.

In the context of synthetic investigations into the preparation of phthalascidin, Z. Z. Liu et al., Chinese Chem. Lett. 13(8) 2002, pp. 701-704 describe an intermediate of the formula below, in which Bn is benzyl:

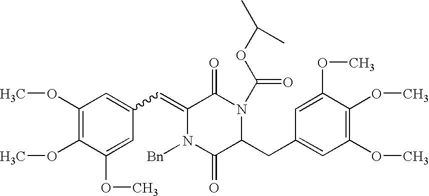

J. Bryans et al., Journal of Antibiotics 49(10), 1996, pp. 1014-1021 describe the compound of the formula below:

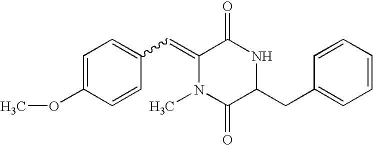

WO 99/48889, WO 01/53290 and WO 2005/011699 describe 2,5-diketopiperazine compounds having in one of the 3- and 6-positions a 4-imidazolyl radical which is attached via a methylene or methyne group and in the other 3- or 6-position a benzyl or benzylidene radical. These compounds have antitumor activity.

The earlier patent application PCT/EP2007/050067 (=WO 2007/077247) describes 2,5-diketopiperazine compounds which have an aryl or hetaryl radical attached via a methyne group in the 3-position and an aryl or hetaryl radical attached via a methylene group in the 6-position.

It is an object of the present invention to provide compounds having herbicidal action. To be provided are in particular compounds which have high herbicidal activity, in particular even at low application rates, and which are sufficiently compatible with crop plants for commercial utilization.

These and further objects are achieved by the compounds of the formula I, defined below, and by their agriculturally suitable salts.

Accordingly, the present invention provides piperazine compounds of the general formula I

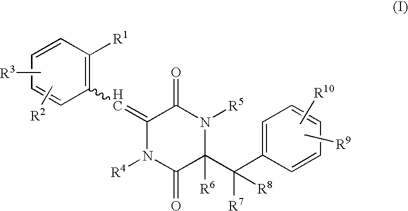

in which
R¹ is selected from the group consisting of halogen, cyano, nitro, Z—C(═O)—R¹¹, phenyl and a 5- or 6-membered heterocyclic radical which has 1, 2, 3 or 4 heteroatoms selected from the group consisting of O, N and S as ring atoms, where phenyl and the heterocyclic radical are unsubstituted or may have 1, 2, 3 or 4 substituents R^{1a} independently of one another selected from the group consisting of halogen, CN, NO₂, C₁-C₄-haloalkyl, C₁-C₄-alkoxy and C₁-C₄-haloalkoxy, and in which
   Z is a covalent bond or a CH₂ group;
   R¹¹ is hydrogen, C₁-C₆-alkyl, C₃-C₆-cycloalkyl, C₂-C₆-alkenyl, C₅-C₆-cycloalkenyl, C₂-C₆-alkynyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₁-C₆-alkoxyamino, C₁-C₆-alkylsulfonylamino, C₁-C₆-alkylaminosulfonylamino, [di-(C₁-C₆)-alkylamino]sulfonylamino, C₃-C₆-alkenylamino, C₃-C₆-alkynylamino, N—(C₂-C₆-alkenyl)-N—(C₁-C₆-alkyl)-amino, N—(C₂-C₆-alkynyl)-N—(C₁-C₆-alkyl)-amino, N—(C₁-C₆-alkoxy)-N—(C₁-C₆-alkyl)amino, N—(C₂-C₆-alkenyl)-N—(C₁-C₆-alkoxy)-amino, N—(C₂-C₆-alkynyl)-N—(C₁-C₆-alkoxy)-amino, phenyl, phenoxy or phenylamino;
   where the alkyl moieties in the radicals listed under R¹¹ may be partially or fully halogenated and the phenyl moieties in the radicals listed under R¹¹ may carry 1, 2, 3 or 4 substituents R^{11a} selected from the group consisting of halogen, CN, NO₂, C₁-C₄-alkyl, C₁-C₄-haloalkyl, C₁-C₄-alkoxy and C₁-C₄-haloalkoxy;
R² is hydrogen, cyano, nitro, halogen, C₁-C₄-alkyl, C₁-C₄-haloalkyl, C₂-C₄-alkenyl, C₁-C₄-alkoxy, C₁-C₄-haloalkoxy, benzyl or a group S(O)ₙR²¹ in which R²¹ is C₁-C₄-alkyl or C₁-C₄-haloalkyl and n is 0, 1 or 2;
R³ is hydrogen or halogen;
R⁴ is C₁-C₄-alkyl, C₃-C₄-alkenyl or C₃-C₄-alkynyl;
R⁵ is hydrogen, C₁-C₄-alkyl, C₃-C₄-alkenyl, C₃-C₄-alkynyl or a group C(═O)R⁵¹ in which R⁵¹ is hydrogen, C₁-C₄-alkyl, C₁-C₄-haloalkyl, C₁-C₄-alkoxy or C₁-C₄-haloalkoxy;
R⁶ is C₁-C₄-alkyl, C₁-C₄-hydroxyalkyl or C₁-C₄-haloalkyl;
R⁷, R⁸ independently of one another are hydrogen, OH, C₁-C₄-alkoxy, C₁-C₄-haloalkyloxy, C₁-C₄-alkyl or C₁-C₄-haloalkyl;
R⁹, R¹⁰ independently of one another are selected from the group consisting of hydrogen, halogen, CN, NO₂, C₁-C₄-alkyl, C₂-C₄-alkenyl, C₁-C₄-alkoxy and C₁-C₄-haloalkoxy;
   and the agriculturally useful salts of these compounds.

The present invention also provides the use of piperazine compounds of the general formula I or the agriculturally useful salts of piperazine compounds of the formula I as herbicides, i.e. for controlling harmful plants.

The present invention also provides compositions comprising at least one piperazine compound of the formula I or an agriculturally useful salt of I and auxiliaries customary for formulating crop protection agents.

The present invention furthermore provides a method for controlling unwanted vegetation where a herbicidally effective amount of at least one piperazine compound of the formula I or an agriculturally useful salt of I is allowed to act on plants, their seeds and/or their habitat.

Moreover, the invention relates to processes and intermediates for preparing compounds of the formula I.

Further embodiments of the present invention are evident from the claims, the description and the examples. It is to be understood that the features mentioned above and still to be illustrated below of the subject matter of the invention can be applied not only in the combination given in each particular case but also in other combinations, without leaving the scope of the invention.

The compounds of the formula I have a center of chirality at the carbon atom which carries the radical R⁶. Depending on the substitution pattern, they may comprise one or more further centers of chirality. Accordingly, the compounds according to the invention may be present as pure enantiomers or diastereomers or as enantiomer or diastereomer mixtures. The invention provides both the pure enantiomers or diastereomers and their mixtures.

The compounds of the formula I may be present as E isomer or Z isomer with respect to the exocyclic double bond. The invention provides both the pure E isomers and Z isomers and their mixtures.

The compounds of the formula I may also be present in the form of their agriculturally useful salts, the nature of the salt generally being immaterial. Suitable salts are, in general, the salts of those cations or the acid addition salts of those acids whose cations and anions, respectively, which have no adverse effect on the herbicidal action of the compounds I.

Suitable cations are in particular ions of the alkali metals, preferably lithium, sodium and potassium, of the alkaline earth metals, preferably calcium and magnesium, and of the transition metals, preferably manganese, copper, zinc and iron, and also ammonium, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-yl-ammonium, di(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and the anions of C₁-C₄-alkanoic acids, preferably formate, acetate, propionate and butyrate.

The organic moieties mentioned for the substituents of the compounds according to the invention are collective terms for individual enumerations of the specific group members. All hydrocarbon chains, such as alkyl, haloalkyl, alkenyl, alkynyl, and also the alkyl moieties and alkenyl moieties in alkoxy, haloalkoxy, alkylamino, dialkylamino, N-alkylsulfonylamino, alkenyloxy, alkynyloxy, alkoxyamino, alkylaminosulfonylamino, dialkylaminosulfonylamino, alkenylamino, alkynylamino, N-(alkenyl)-N-(alkyl)-amino, N-(alkynyl)-N-(alkyl)-amino, N-(alkoxy)-N-(alkyl)-amino, N-(alkenyl)-N-(alkoxy)-amino or N-(alkynyl)-N-(alkoxy)-amino may be straight-chain or branched.

The prefix Cₙ-Cₘ— indicates the respective carbon number of the hydrocarbon moiety. Unless indicated otherwise, halogenated substituents preferably carry one to five identical or different halogen atoms, in particular fluorine atoms or chlorine atoms.

The term halogen denotes in each case fluorine, chlorine, bromine or iodine.

Examples of other meanings are:

alkyl and also the alkyl moieties, for example, in alkoxy, alkylamino, dialkylamino, N-alkylsulfonylamino, alkylaminosulfonylamino, dialkylaminosulfonylamino, N-(alkenyl)-N-(alkyl)-amino, N-(alkynyl)-N-(alkyl)-amino, N-(alkoxy)-N-(alkyl)-amino: saturated straight-chain or branched hydrocarbon radicals having one or more carbon atoms, for example 1 to 2, 1 to 4 or 1 to 6 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. In one embodiment according to the invention, alkyl denotes small alkyl groups such as C₁-C₄-alkyl. In another embodiment according to the invention, alkyl denotes relatively large alkyl groups such as C₅-C₆-alkyl.

Haloalkyl: an alkyl radical as mentioned above whose hydrogen atoms are partially or fully substituted by halogen atoms such as fluorine, chlorine, bromine and/or iodine, for example chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl and nonafluorobutyl.

Cycloalkyl and also the cycloalkyl moieties, for example, in cycloalkoxy or cycloalkylcarbonyl: monocyclic saturated hydrocarbon groups having three or more carbon atoms, for example 3 to 6 carbon ring members, such as cyclopropyl, cyclobutyl, cyclopentyl and cyclohexyl.

Alkenyl and also alkenyl moieties, for example in alkenylamino, alkenyloxy, N-(alkenyl)-N-(alkyl)-amino, N-(alkenyl)-N-(alkoxy)-amino: monounsaturated straight-chain or branched hydrocarbon radicals having two or more carbon atoms, for example 2 to 4, 2 to 6, or 3 to 6 carbon atoms, and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl.

Cycloalkenyl: monocyclic, monounsaturated hydrocarbon groups having from 5 to 6, preferably 5 to 6, carbon ring members, such as cyclopenten-1-yl, cyclopenten-3-yl, cyclohexen-1-yl, cyclohexen-3-yl, cyclohexen-4-yl.

Alkynyl and also alkynyl moieties, for example in alkynyloxy, alkynylamino, N-(alkynyl)-N-(alkyl)-amino or N-(alkynyl)-N-(alkoxy)-amino: straight-chain or branched hydrocarbon groups having two or more carbon atoms, for example 2 to 4, 2 to 6, or 3 to 6 carbon atoms, and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl.

Alkoxy: alkyl, as defined above, which is attached via an oxygen atom: for example methoxy, ethoxy, n-propoxy, 1-methylethoxy, butoxy, 1-methylpropoxy, 2-methylpropoxy or 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

Aryl: monocyclic or polycyclic aromatic hydrocarbon radicals having 6 to 14 carbon atoms, such as phenyl, naphthyl, anthracenyl or phenanthrenyl, preferably phenyl or naphthyl.

A 5- or 6-membered heterocyclic radical: a heterocyclic radical which has 5 or 6 ring atoms, 1, 2, 3 or 4 ring atoms being heteroatoms selected from the group consisting of O, S and N, where the heterocyclic radical is saturated, partially unsaturated or aromatic. Examples of heterocyclic radicals are:
5-membered saturated rings attached via carbon, such as
tetrahydrofuran-2-yl, tetrahydrofuran-3-yl, tetrahydrothien-2-yl, tetrahydrothien-3-yl, tetrahydropyrrol-2-yl, tetrahydropyrrol-3-yl, tetrahydropyrazol-3-yl, tetrahydropyrazol-4-yl, tetrahydroisoxazol-3-yl, tetrahydroisoxazol-4-yl, tetrahydroisoxazol-5-yl, 1,2-oxathiolan-3-yl, 1,2-oxathiolan-4-yl, 1,2-oxathiolan-5-yl, tetrahydroisothiazol-3-yl, tetrahydroisothiazol-4-yl, tetrahydroisothiazol-5-yl, 1,2-dithiolan-3-yl, 1,2-dithiolan-4-yl, tetrahydroimidazol-2-yl, tetrahydroimidazol-4-yl, tetrahydrooxazol-2-yl, tetrahydrooxazol-4-yl, tetrahydrooxazol-5-yl, tetrahydrothiazol-2-yl, tetrahydrothiazol-4-yl, tetrahydrothiazol-5-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-dithiolan-2-yl, 1,3-dithiolan-4-yl, 1,3,2-dioxathiolan-4-yl;
6-membered saturated rings attached via carbon, such as:
   tetrahydropyran-2-yl, tetrahydropyran-3-yl, tetrahydropyran-4-yl, piperidin-2-yl, piperidin-3-yl, piperidin-4-yl, tetrahydrothiopyran-2-yl, tetrahydrothiopyran-3-yl, tetrahydrothiopyran-4-yl, 1,3-dioxan-2-yl, 1,3-dioxan-4-yl, 1,3-dioxan-5-yl, 1,4-dioxan-2-yl, 1,3-dithian-2-yl, 1,3-dithian-4-yl, 1,3-dithian-5-yl, 1,4-dithian-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, 1,2-dithian-3-yl, 1,2-dithian-4-yl, hexahydropyrimidin-2-yl, hexahydropyrimidin-4-yl, hexahydropyrimidin-5-yl, hexahydropyrazin-2-yl, hexahydropyridazin-3-yl, hexahydropyridazin-4-yl, tetrahydro-1,3-oxazin-2-yl, tetrahydro-1,3-oxazin-4-yl, tetrahydro-1,3-oxazin-5-yl, tetrahydro-1,3-oxazin-6-yl, tetrahydro-1,3-thiazin-2-yl, tetrahydro-1,3-thiazin-4-yl, tetrahydro-1,3-thiazin-5-yl, tetrahydro-1,3-thiazin-6-yl, tetrahydro-1,4-thiazin-2-yl, tetrahydro-1,4-thiazin-3-yl, tetrahydro-1,4-oxazin-2-yl, tetrahydro-1,4-oxazin-3-yl, tetrahydro-1,2-oxazin-3-yl, tetrahydro-1,2-oxazin-4-yl, tetrahydro-1,2-oxazin-5-yl, tetrahydro-1,2-oxazin-6-yl;
5-membered saturated rings attached via nitrogen, such as:
tetrahydropyrrol-1-yl, tetrahydropyrazol-1-yl, tetrahydroisoxazol-2-yl, tetrahydroisothiazol-2-yl, tetrahydroimidazol-1-yl, tetrahydrooxazol-3-yl, tetrahydrothiazol-3-yl;
6-membered saturated rings attached via nitrogen, such as:
piperidin-1-yl, hexahydropyrimidin-1-yl, hexahydropyrazin-1-yl, hexahydropyridazin-1-yl, tetrahydro-1,3-oxazin-3-yl, tetrahydro-1,3-thiazin-3-yl, tetrahydro-1,4-thiazin-4-yl, tetrahydro-1,4-oxazin-4-yl, tetrahydro-1,2-oxazin-2-yl;
5-membered partially unsaturated rings attached via carbon, such as:
2,3-dihydrofuran-2-yl, 2,3-dihydrofuran-3-yl, 2,5-dihydrofuran-2-yl, 2,5-di-hydrofuran-3-yl, 4,5-dihydrofuran-2-yl, 4,5-dihydrofuran-3-yl, 2,3-dihydrothien-2-yl, 2,3-dihydrothien-3-yl, 2,5-dihydrothien-2-yl, 2,5-dihydrothien-3-yl, 4,5-dihydrothien-2-yl, 4,5-dihydrothien-3-yl, 2,3-dihydro-1H-pyrrol-2-yl, 2,3-dihydro-1H-pyrrol-3-yl, 2,5-dihydro-1H-pyrrol-2-yl, 2,5-dihydro-1H-pyrrol-3-yl, 4,5-dihydro-1H-pyrrol-2-yl, 4,5-dihydro-1H-pyrrol-3-yl, 3,4-dihydro-2H-pyrrol-2-yl, 3,4-dihydro-2H-pyrrol-3-yl, 3,4-dihydro-5H-pyrrol-2-yl, 3,4-dihydro-5H-pyrrol-3-yl, 4,5-dihydro-1H-pyrazol-3-yl, 4,5-dihydro-1H-pyrazol-4-yl, 4,5-dihydro-1H-pyrazol-5-yl, 2,5-dihydro-1H-pyrazol-3-yl, 2,5-dihydro-1H-pyrazol-4-yl, 2,5-dihydro-1H-pyrazol-5-yl, 4,5-dihydroisoxazol-3-yl, 4,5-dihydroisoxazol-4-yl, 4,5-dihydroisoxazol-5-yl, 2,5-dihydroisoxazol-3-yl, 2,5-dihydroisoxazol-4-yl, 2,5-dihydroisoxazol-5-yl, 2,3-dihydroisoxazol-3-yl, 2,3-dihydroisoxazol-4-yl, 2,3-dihydroisoxazol-5-yl, 4,5-dihydroisothiazol-3-yl, 4,5-dihydroisothiazol-4-yl, 4,5-dihydroisothiazol-5-yl, 2,5-dihydroisothiazol-3-yl, 2,5-dihydroisothiazol-4-yl, 2,5-dihydroisothiazol-5-yl, 2,3-dihydroisothiazol-3-yl, 2,3-dihydroisothiazol-4-yl, 2,3-dihydroisothiazol-5-yl, Δ³-1,2-dithiol-3-yl, Δ³-1,2-dithiol-4-yl, Δ³-1,2-dithiol-5-yl, 4,5-dihydro-1H-imidazol-2-yl, 4,5-dihydro-1H-imidazol-4-yl, 4,5-dihydro-1H-imidazol-5-yl, 2,5-dihydro-1H-imidazol-2-yl, 2,5-dihydro-1H-imidazol-4-yl, 2,5-dihydro-1H-imidazol-5-yl, 2,3-dihydro-1H-imidazol-2-yl, 2,3-dihydro-1H-imidazol-4-yl, 4,5-dihydrooxazol-2-yl, 4,5-dihydrooxazol-4-yl, 4,5-dihydrooxazol-5-yl, 2,5-dihydrooxazol-2-yl, 2,5-dihydrooxazol-4-yl, 2,5-dihydrooxazol-5-yl, 2,3-dihydrooxazol-2-yl, 2,3-dihydrooxazol-4-yl, 2,3-dihydrooxazol-5-yl, 4,5-dihydrothiazol-2-yl, 4,5-dihydrothiazol-4-yl, 4,5-dihydrothiazol-5-yl, 2,5-dihydrothiazol-2-yl, 2,5-dihydrothiazol-4-yl, 2,5-dihydrothiazol-5-yl, 2,3-dihydrothiazol-2-yl, 2,3-dihydrothiazol-4-yl, 2,3-dihydrothiazol-5-yl, 1,3-dioxol-2-yl, 1,3-dioxol-4-yl, 1,3-dithiol-2-yl, 1,3-dithiol-4-yl, 1,3-oxathiol-2-yl, 1,3-oxathiol-4-yl, 1,3-oxathiol-5-yl;
6-membered partially unsaturated rings attached via carbon, such as:
2H-3,4-dihydropyran-6-yl, 2H-3,4-dihydropyran-5-yl, 2H-3,4-dihydropyran-4-yl, 2H-3,4-dihydropyran-3-yl, 2H-3,4-dihydropyran-2-yl, 2H-3,4-dihydropyran-6-yl, 2H-3,4-dihydrothiopyran-5-yl, 2H-3,4-dihydrothiopyran-4-yl, 2H-3,4-dihydropyran-3-yl, 2H-3,4-dihydropyran-2-yl, 1,2,3,4-tetrahydropyridin-6-yl, 1,2,3,4-tetrahydropyridin-5-yl, 1,2,3,4-tetrahydropyridin-4-yl, 1,2,3,4-tetrahydropyridin-3-yl, 1,2,3,4-tetrahydropyridin-2-yl, 2H-5,6-dihydropyran-2-yl, 2H-5,6-dihydropyran-3-yl, 2H-5,6-dihydropyran-4-yl, 2H-5,6-dihydropyran-5-yl, 2H-5,6-dihydropyran-6-yl, 2H-5,6-dihydrothiopyran-2-yl, 2H-5,6-dihydrothiopyran-3-yl, 2H-5,6-dihydrothiopyran-4-yl, 2H-5,6-dihydrothiopyran-5-yl, 2H-5,6-dihydrothiopyran-6-yl, 1,2,5,6-tetrahydropyridin-2-yl, 1,2,5,6-tetrahydropyridin-3-yl, 1,2,5,6-tetrahydropyridin-4-yl, 1,2,5,6-tetrahydropyridin-5-yl, 1,2,5,6-tetrahydropyridin-6-yl, 2,3,4,5-tetrahydropyridin-2-yl, 2,3,4,5-tetrahydropyridin-3-yl, 2,3,4,5-tetrahydropyridin-4-yl, 2,3,4,5-tetrahydropyridin-5-yl, 2,3,4,5-tetrahydropyridin-6-yl, 4H-pyran-2-yl, 4H-pyran-3-yl, 4H-pyran-4-yl, 4H-thiopyran-2-yl, 4H-thiopyran-3-yl, 4H-thiopyran-4-yl, 1,4-dihydropyridin-2-yl, 1,4-dihydropyridin-3-yl, 1,4-dihydropyridin-4-yl, 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 3,4-dihydropyridin-2-yl, 3,4-dihydropyridin-3-yl, 3,4-dihydropyridin-4-yl, 3,4-dihydropyridin-5-yl, 3,4-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2H-5,6-dihydro-1,2-oxazin-3-yl, 2H-5,6-dihydro-1,2-oxazin-4-yl, 2H-5,6-dihydro-1,2-oxazin-5-yl, 2H-5,6-dihydro-1,2-oxazin-6-yl, 2H-5,6-dihydro-1,2-thiazin-3-yl, 2H-5,6-dihydro-1,2-thiazin-4-yl, 2H-5,6-dihydro-1,2-thiazin-5-yl, 2H-5,6-dihydro-1,2-thiazin-6-yl, 4H-5,6-dihydro-1,2-oxazin-3-yl, 4H-5,6-dihydro-1,2-oxazin-4-yl, 4H-5,6-dihydro-1,2-oxazin-5-yl, 4H-5,6-dihydro-1,2-oxazin-6-yl, 4H-5,6-dihydro-1,2-thiazin-3-yl, 4H-5,6-dihydro-1,2-thiazin-4-yl, 4H-5,6-dihydro-1,2-thiazin-5-yl, 4H-5,6-dihydro-1,2-thiazin-6-yl, 2H-3,6-dihydro-1,2-oxazin-3-yl, 2H-3,6-dihydro-1,2-oxazin-4-yl, 2H-3,6-dihydro-1,2-oxazin-5-yl, 2H-3,6-dihydro-1,2-oxazin-6-yl, 2H-3,6-dihydro-1,2-thiazin-3-yl, 2H-3,6-dihydro-1,2-thiazin-4-yl, 2H-3,6-di-hydro-1,2-thiazin-5-yl, 2H-3,6-dihydro-1,2-thiazin-6-yl, 2H-3,4-dihydro-1,2-oxazin-3-yl, 2H-3,4-dihydro-1,2-oxazin-4-yl, 2H-3,4-dihydro-1,2-oxazin-5-yl, 2H-3,4-dihydro-1,2-oxazin-6-yl, 2H-3,4-dihydro-1,2-thiazin-3-yl, 2H-3,4-dihydro-1,2-thiazin-4-yl, 2H-3,4-dihydro-1,2-thiazin-5-yl, 2H-3,4-dihydro-1,2-thiazin-6-yl, 2,3,4,5-tetrahydropyridazin-3-yl, 2,3,4,5-tetrahydropyridazin-4-yl, 2,3,4,5-tetrahydropyridazin-5-yl, 2,3,4,5-tetrahydropyridazin-6-yl, 3,4,5,6-tetrahydropyridazin-3-yl, 3,4,5,6-tetrahydropyridazin-4-yl, 1,2,5,6-tetrahydropyridazin-3-yl, 1,2,5,6-tetrahydropyridazin-4-yl, 1,2,5,6-tetrahydropyridazin-5-yl, 1,2,5,6-tetrahydropyridazin-6-yl, 1,2,3,6-tetrahydropyridazin-3-yl, 1,2,3,6-tetrahydropyridazin-4-yl, 4H-5,6-dihydro-1,3-oxazin-2-yl, 4H-5,6-dihydro-1,3-oxazin-4-yl, 4H-5,6-dihydro-1,3-oxazin-5-yl, 4H-5,6-dihydro-1,3-oxazin-6-yl, 4H-5,6-dihydro-1,3-thiazin-2-yl, 4H-5,6-dihydro-1,3-thiazin-4-yl, 4H-5,6-dihydro-1,3-thiazin-5-yl, 4H-5,6-dihydro-1,3-thiazin-6-yl, 3,4,5-6-tetrahydropyrimidin-2-yl, 3,4,5,6-tetrahydropyrimidin-4-yl, 3,4,5,6-tetrahydropyrimidin-5-yl, 3,4,5,6-tetrahydropyrimidin-6-yl, 1,2,3,4-tetrahydropyrazin-2-yl, 1,2,3,4-tetrahydropyrazin-5-yl, 1,2,3,4-tetrahydropyrimidin-2-yl, 1,2,3,4-tetrahydropyrimidin-4-yl, 1,2,3,4-tetrahydropyrimidin-5-yl, 1,2,3,4-tetrahydropyrimidin-6-yl, 2,3-dihydro-1,4-thiazin-2-yl, 2,3-dihydro-1,4-thiazin-3-yl, 2,3-dihydro-1,4-thiazin-5-yl, 2,3-dihydro-1,4-thiazin-6-yl, 2H-1,2-oxazin-3-yl, 2H-1,2-oxazin-4-yl, 2H-1,2-oxazin-5-yl, 2H-1,2-oxazin-6-yl, 2H-1,2-thiazin-3-yl, 2H-1,2-thiazin-4-yl, 2H-1,2-thiazin-5-yl, 2H-1,2-thiazin-6-yl, 4H-1,2-oxazin-3-yl, 4H-1,2-oxazin-4-yl, 4H-1,2-oxazin-5-yl, 4H-1,2-oxazin-6-yl, 4H-1,2-thiazin-3-yl, 4H-1,2-thiazin-4-yl, 4H-1,2-thiazin-5-yl, 4H-1,2-thiazin-6-yl, 6H-1,2-oxazin-3-yl, 6H-1,2-oxazin-4-yl, 6H-1,2-oxazin-5-yl, 6H-1,2-oxazin-6-yl, 6H-1,2-thiazin-3-yl, 6H-1,2-thiazin-4-yl, 6H-1,2-thiazin-5-yl, 6H-1,2-thiazin-6-yl, 2H-1,3-oxazin-2-yl, 2H-1,3-oxazin-4-yl, 2H-1,3-oxazin-5-yl, 2H-1,3-oxazin-6-yl, 2H-1,3-thiazin-2-yl, 2H-1,3-thiazin-4-yl, 2H-1,3-thiazin-5-yl, 2H-1,3-thiazin-6-yl, 4H-1,3-oxazin-2-yl, 4H-1,3-oxazin-4-yl, 4H-1,3-oxazin-5-yl, 4H-1,3-oxazin-6-yl, 4H-1,3-thiazin-2-yl, 4H-1,3-thiazin-4-yl, 4H-1,3-thiazin-5-yl, 4H-1,3-thiazin-6-yl, 6H-1,3-oxazin-2-yl, 6H-1,3-oxazin-4-yl, 6H-1,3-oxazin-5-yl, 6H-1,3-oxazin-6-yl, 6H-1,3-thiazin-2-yl, 6H-1,3-oxazin-4-yl, 6H-1,3-oxazin-5-yl, 6H-1,3-thiazin-6-yl, 2H-1,4-oxazin-2-yl, 2H-1,4-oxazin-3-yl, 2H-1,4-oxazin-5-yl, 2H-1,4-oxazin-6-yl, 2H-1,4-thiazin-2-yl, 2H-1,4-thiazin-3-yl, 2H-1,4-thiazin-5-yl, 2H-1,4-thiazin-6-yl, 4H-1,4-oxazin-2-yl, 4H-1,4-oxazin-3-yl, 4H-1,4-thiazin-2-yl, 4H-1,4-thiazin-3-yl, 1,4-dihydropyridazin-3-yl, 1,4-dihydropyridazin-4-yl, 1,4-dihydropyridazin-5-yl, 1,4-dihydropyridazin-6-yl, 1,4-dihydropyrazin-2-yl, 1,2-dihydropyrazin-2-yl, 1,2-dihydropyrazin-3-yl, 1,2-dihydropyrazin-5-yl, 1,2-dihydropyrazin-6-yl, 1,4-dihydropyrimidin-2-yl, 1,4-dihydropyrimidin-4-yl, 1,4-dihydropyrimidin-5-yl, 1,4-dihydropyrimidin-6-yl, 3,4-dihydropyrimidin-2-yl, 3,4-dihydropyrimidin-4-yl, 3,4-dihydropyrimidin-5-yl or 3,4-dihydropyrimidin-6-yl;
5-membered partially unsaturated rings attached via nitrogen, such as:
2,3-dihydro-1H-pyrrol-1-yl, 2,5-dihydro-1H-pyrrol-1-yl, 4,5-dihydro-1H-pyrazol-1-yl, 2,5-dihydro-1H-pyrazol-1-yl, 2,3-dihydro-1H-pyrazol-1-yl, 2,5-dihydroisoxazol-2-yl, 2,3-dihydroisoxazol-2-yl, 2,5-dihydroisothiazol-2-yl, 2,3-dihydroisoxazol-2-yl, 4,5-dihydro-1H-imidazol-1-yl, 2,5-dihydro-1H-imidazol-1-yl, 2,3-dihydro-1H-imidazol-1-yl, 2,3-dihydrooxazol-3-yl, 2,3-dihydrothiazol-3-yl, 1,2,4-Δ⁴-oxadiazolin-2-yl, 1,2,4-Δ²-oxadiazolin-4-yl, 1,2,4-Δ³-oxadiazolin-2-yl, 1,3,4-Δ²-oxadiazolin-4-yl, 1,2,4-Δ⁵-thiadiazolin-2-yl, 1,2,4-Δ³-thiadiazolin-2-yl, 1,2,4-Δ²-thiadiazolin-4-yl, 1,3,4-Δ²-thiadiazolin-4-yl, 1,2,3-Δ²-triazolin-1-yl, 1,2,4-Δ²-triazolin-1-yl, 1,2,4-Δ²-triazolin-4-yl, 1,2,4-Δ³-triazolin-1-yl, 1,2,4-Δ¹-triazolin-4-yl;
6-membered partially unsaturated rings attached via nitrogen, such as:
1,2,3,4-tetrahydropyridin-1-yl, 1,2,5,6-tetrahydropyridin-1-yl, 1,4-dihydropyridin-1-yl, 1,2-dihydropyridin-1-yl, 2H-5,6-dihydro-1,2-oxazin-2-yl, 2H-5,6-dihydro-1,2-thiazin-2-yl, 2H-3,6-dihydro-1,2-oxazin-2-yl, 2H-3,6-dihydro-1,2-thiazin-2-yl, 2H-3,4-dihydro-1,2-oxazin-2-yl, 2H-3,4-dihydro-1,2-thiazin-2-yl, 2,3,4,5-tetrahydropyridazin-2-yl, 1,2,5,6-tetrahydropyridazin-1-yl, 1,2,5,6-tetrahydropyridazin-2-yl, 1,2,3,6-tetrahydropyridazin-1-yl, 3,4,5,6-tetrahydropyrimidin-3-yl, 1,2,3,4-tetrahydropyrazin-1-yl, 1,2,3,4-tetrahydropyrimidin-1-yl, 1,2,3,4-tetrahydropyrimidin-3-yl, 2,3-dihydro-1,4-thiazin-4-yl, 2H-1,2-oxazin-2-yl, 2H-1,2-thiazin-2-yl, 4H-1,4-oxazin-4-yl, 4H-1,4-thiazin-4-yl, 1,4-dihydropyridazin-1-yl, 1,4-dihydropyrazin-1-yl, 1,2-dihydropyrazin-1-yl, 1,4-dihydropyrimidin-1-yl or 3,4-dihydropyrimidin-3-yl;
5-membered heteroaromatic rings attached via carbon, such as:
2-furyl, 3-furyl, 2-thienyl, 3-thienyl, pyrrol-2-yl, pyrrol-3-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-2-yl, imidazol-4-yl, oxazol-2-yl, oxazol-4-yl, oxazol-5-yl, thiazol-2-yl, thiazol-4-yl, thiazol-5-yl, 1,2,3-oxadiazol-4-yl, 1,2,3-oxadiazol-5-yl, 1,2,4-oxadiazol-3-yl, 1,2,4-oxadiazol-5-yl, 1,3,4-oxadiazol-2-yl, 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl, 1,2,3-triazol-4-yl, 1,2,4-triazol-3-yl, [1H]-tetrazol-5-yl and [21-1]-tetrazol-5-yl;
6-membered heteroaromatic rings attached via carbon, such as:
pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1,2,4-triazin-3-yl, 1,2,4-triazin-5-yl and 1,2,4-triazin-6-yl;
5-membered heteroaromatic rings attached via nitrogen, such as:
pyrrol-1-yl, pyrazol-1-yl, imidazol-1-yl, 1,2,3-triazol-1-yl, 1,2,4-triazol-1-yl, [1H]-tetrazol-1-yl and [2H]-tetrazol-2-yl.

The heterocycles mentioned above may be substituted in the manner stated. Sulfur atoms in the heterocycles mentioned above may be oxidized to S═O or S(═O)₂.

Other meanings are:
alkenyloxy: alkenyl as mentioned above which is attached via an oxygen atom;
alkynyloxy: alkynyl as mentioned above which is attached via an oxygen atom;
alkylamino: a group NHR in which R is alkyl as defined above;
[dialkyl]amino: a group NR′R in which R and R′ are alkyl as defined above;
alkoxyamino: a group NH(OR) in which R is alkyl as defined above;
alkylsulfonylamino: a group NHS(O)₂R;
alkylaminosulfonylamino: a group NHS(O)₂NHR in which R is alkyl as defined above;
[dialkylamino]sulfonylamino: a group NHS(O)₂NR′R in which R and R′ are alkyl as defined above;
alkenylamino: a group NHR in which R is alkenyl as defined above;
alkenylamino: a group NHR in which R is alkynyl as defined above;
N-(alkenyl)-N-(alkyl)-amino: a group NR′R in which R is alkenyl and R′ is alkyl as defined above;
N-(alkynyl)-N-(alkyl)-amino: a group NR′R in which R is alkynyl and R′ is alkyl as defined above;
N-(alkoxy)-N-(alkyl)-amino: a group NR′R in which R is alkyl and R′ is alkoxy as defined above;
N-(alkenyl)-N-(alkoxy)-amino: a group NR′R in which R is alkenyl and R′ is alkoxy as defined above; and
N-(alkynyl)-N-(alkoxy)-amino: a group NR′R in which R is alkynyl and R′ is alkoxy as defined above.

In a particular embodiment, the variables of the compounds of the formula I have the meanings below, these meanings—both on their own and in combination with one another—being particular embodiments of the compounds of the formula I:

R¹ is in particular cyano, nitro or a 5- or 6-membered heteroaromatic radical as defined above which preferably has either 1, 2, 3 or 4 nitrogen atoms or 1 oxygen or 1 sulfur atom and, if appropriate, 1 or 2 nitrogen atoms as ring members and which is unsubstituted or may have 1 or 2 substituents selected from R^{1a}.

In a first preferred embodiment of the invention, R¹ is cyano or nitro.

In a further preferred embodiment of the invention, R¹ is a 5- or 6-membered heteroaromatic radical as defined above which preferably has either 1, 2, 3 or 4 nitrogen atoms or 1 oxygen or 1 sulfur atom and, if appropriate, 1 or 2 nitrogen atoms as ring members and which is unsubstituted or may have 1 or 2 substituents selected from R^{1a}. Examples of preferred heteroaromatic radicals are pyridazin-3-yl, pyridazin-4-yl, pyrimidin-2-yl, pyrimidin-4-yl, pyrimidin-5-yl, pyrazin-2-yl, 2-furyl, 3-furyl, 2-thienyl, 3-thienyl, pyrazol-1-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-1-yl, imidazol-2-yl, imidazol-4-yl, imidazol-5-yl, oxazol-2-yl, oxazol-4-yl, oxazol-5-yl, thiazol-2-yl, thiazol-4-yl and thiazol-5-yl, in particular heteroaromatic radicals attached via carbon, such as pyrazol-3-yl, imidazol-5-yl, oxazol-2-yl, thiazol-2-yl, thiazol-4-yl, thiazol-5-yl, pyridin-2-yl, pyridin-3-yl, pyridin-4-yl, pyrimidin-2-yl, pyrimidin-4-yl, pyrimidin-5-yl, pyridazin-4-yl, pyrazin-2-yl, [1H]-tetrazol-5-yl and [2H]-tetrazol-5-yl, where the heterocycles mentioned here in an exemplary manner may have 1 or 2 substituents selected from R^{1a}. Preferred radicals R^{1a} are in particular F, Cl, CN, nitro, methyl, ethyl, methoxy, ethoxy, difluoromethoxy, trifluoromethoxy and trifluoromethyl.

Preference is likewise given to compounds of the general formula I and salts thereof in which R¹ is halogen, in particular chlorine or bromine.

The radical R² is preferably hydrogen, fluorine, chlorine, C₁-C₂-alkyl, C₁-C₂-fluoroalkyl, ethenyl, C₁-C₂-alkoxy or C₁-C₂-fluoroalkoxy, in particular fluorine, chlorine, methyl, ethyl, methoxy, ethenyl or trifluoromethoxy. R² is especially preferably hydrogen, fluorine or chlorine.

From among the compounds of the formula I in which R² is different from hydrogen, preference is given to those compounds in which R² is located in the ortho-position to the point of attachment of the phenyl ring.

In a particularly preferred embodiment, R² is halogen, in particular chlorine or fluorine, which is located in the ortho-position to the point of attachment of the phenyl ring.

From among the compounds of the formula I in which R³ is halogen, preference is given to those compounds in which R³ is located in the para-position to the group R¹.

From among the compounds of the formula I in which R³ is halogen, preference is given to those compounds in which R³ is fluorine or chlorine. In another, likewise preferred, embodiment, R³ is hydrogen.

R⁴ is preferably methyl.

R⁵ is preferably hydrogen, methyl or ethyl, especially methyl.

Preference is likewise given to compounds of the formula I in which R⁵ is a group C(═O)R⁵¹ in which R⁵¹ has one of the meanings given above and is in particular hydrogen, C₁-C₄-alkyl, especially methyl or ethyl, or C₁-C₄-haloalkyl, especially C₁-C₂-fluoroalkyl, such as trifluoromethyl.

R⁶ is preferably C₁-C₃-alkyl or C₁-C₂-fluoroalkyl, in particular methyl, ethyl, n-propyl, or trifluoromethyl, and especially methyl or ethyl.

Preferably at least one and in particular both radicals R⁷ and R⁸ are hydrogen.

From among the compounds of the formula I in which R⁹ is a radical different from hydrogen, preference is given to those compounds in which R⁹ is located in the para-position to the group CR⁷R⁸.

From among the compounds of the formula I in which R⁹ is a radical different from hydrogen, preference is given to those compounds in which R⁹ is halogen, in particular fluorine or chlorine. In another, likewise preferred, embodiment, R⁹ is hydrogen.

R¹⁰ is preferably hydrogen.

In group C(O)R¹¹, R¹¹ is preferably hydrogen, C₁-C₄-alkyl or C₁-C₄-haloalkyl.

From among the compounds of the formula I and their salts, preference is given to the compounds of the general formula Ia and their agriculturally useful salts:

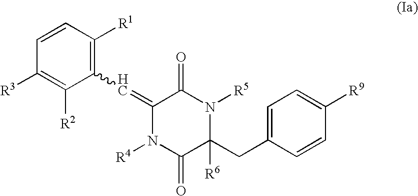

in which R¹, R², R³, R⁴, R⁵, R⁶ and R⁹ have one of the meanings given above, in particular the meanings given as being preferred. In formula Ia the radicals R¹, R², R³, R⁴, R⁵, R⁶ and R⁹ independently of one another, but preferably in combination, have in particular the meanings below:
R¹ cyano or nitro;
R² hydrogen, fluorine, chlorine, C₁-C₂-alkyl, ethenyl or C₁-C₂-alkoxy, in particular hydrogen, fluorine or chlorine;
R³ fluorine or hydrogen;
R⁴ methyl;
R⁵ hydrogen, methyl or ethyl, especially methyl;
R⁶ methyl or ethyl; and
R⁹ hydrogen or halogen, in particular hydrogen or fluorine.

At the carbon atom which carries the group R⁶, the compounds of the formula I have a center of chirality. A preferred embodiment of the invention relates to the pure enantiomers of the formula I-S given below in which R¹, R², R³, R⁴, R⁵, R⁶, R⁷, R⁸, R⁹ and R¹⁰ have one of the meanings given above, in particular one of the meanings given as being preferred or as being particularly preferred, and also to enantiomer mixtures having an enantiomeric excess with respect to the enantiomer of the formula I-S.

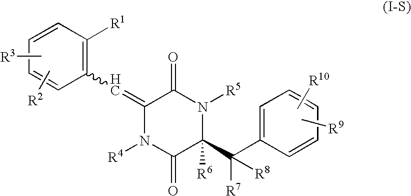

Enantiomeric excess means preferably an ee (enantiomeric excess) of at least 70%, in particular at least 80% and especially at least 90%. Preference is also given to the agriculaturally useful salts of the enantiomers I-S and enantiomer mixtures of the salts having an enantiomeric excess with respect to the enantiomer of the formula I-S.

Another, likewise preferred, embodiment relates to the racemates of I and their salts.

A particularly preferred embodiment relates to the pure enantiomers of the formula I-S.a shown below in which R¹, R², R³, R⁴, R⁵, R⁶ and R⁹ have one of the meanings given above, in particular one of the meanings given as being preferred or as being particularly preferred, and also to enantiomer mixtures having an enantiomeric excess with respect to the enantiomer of the formula I-S.a.

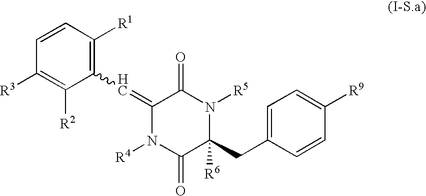

Preference is also given to the agriculturally useful salts of the enantiomers I-S.a and to enantiomer mixtures of the salts having an enantiomeric excess with respect to the enantiomer of the formula I-S.a.

In formula I-S.a, the radicals R¹, R², R³, R⁴, R⁵, R⁶ and R⁹ independently of one another, but preferably in combination, have in particular the meanings below:
R¹ cyano or nitro;
R² hydrogen, fluorine, chlorine, C₁-C₂-alkyl, ethenyl or C₁-C₂-alkoxy, in particular hydrogen, fluorine or chlorine;
R³ fluorine or hydrogen;
R⁴ methyl;
R⁵ hydrogen, methyl or ethyl, especially methyl;
R⁶ methyl or ethyl; and
R⁹ hydrogen or halogen, in particular hydrogen or fluorine.

Another particularly preferred embodiment of the invention relates to the racemates of Ia and salts thereof.

From among the compounds of the formulae I, I.a, I-S and I-S.a, preference is given to those compounds in which the exo double bond at the piperazine ring has the (Z) configuration. Preference is also given to mixtures of the (E) isomer with the (Z) isomer in which the Z isomer is present in excess, in particular to isomer mixtures having an E/Z ratio of not more than 1:2, in particular not more than 1:5.

Examples of compounds which are preferred according to the invention are the compounds mentioned below and their salts:
2-[5-benzyl-1,4,5-trimethyl-3,6-dioxopiperazin-2-ylidenemethyl]benzonitrile,
2-[5-benzyl-1,4,5-trimethyl-3,6-dioxopiperazin-2-ylidenemethyl]-3-fluorobenzonitrile,
2-[5-benzyl-1,4,5-trimethyl-3,6-dioxopiperazin-2-ylidenemethyl]-3-methoxybenzonitrile,
2-[5-benzyl-1,4,5-trimethyl-3,6-dioxopiperazin-2-ylidenemethyl]-3,4-difluorobenzonitrile,
2-[5-benzyl-1,4,5-trimethyl-3,6-dioxopiperazin-2-ylidenemethyl]-3-methylbenzonitrile,
2-[5-benzyl-1,4,5-trimethyl-3,6-dioxopiperazin-2-ylidenemethyl]-3-ethenylbenzonitrile,
2-[5-benzyl-1,5-dimethyl-3,6-dioxopiperazin-2-ylidenemethyl]benzonitrile,
2-[5-benzyl-1,5-dimethyl-3,6-dioxopiperazin-2-ylidenemethyl]-3-fluorobenzonitrile,
2-[5-benzyl-1,5-dimethyl-3,6-dioxopiperazin-2-ylidenemethyl]-3-methoxybenzonitrile,
2-[5-benzyl-1,5-dimethyl-3,6-dioxopiperazin-2-ylidenemethyl]-3,4-difluorobenzonitrile,
2-[5-benzyl-1,5-dimethyl-3,6-dioxopiperazin-2-ylidenemethyl]-3-methylbenzonitrile,
2-[5-benzyl-1,5-dimethyl-3,6-dioxopiperazin-2-ylidenemethyl]-3-ethenylbenzonitrile,
2-[5-benzyl-5-ethyl-1,4-dimethyl-3,6-dioxopiperazin-2-ylidenemethyl]benzonitrile,
2-[5-benzyl-5-ethyl-1,4-dimethyl-3,6-dioxopiperazin-2-ylidenemethyl]-3-fluorobenzonitrile,
2-[5-benzyl-5-ethyl-1,4-dimethyl-3,6-dioxopiperazin-2-ylidenemethyl]-3-methoxy-benzonitrile,
2-[5-benzyl-5-ethyl-1,4-dimethyl-3,6-dioxopiperazin-2-ylidenemethyl]-3,4-difluorobenzonitrile,
2-[5-benzyl-5-ethyl-1,4-dimethyl-3,6-dioxopiperazin-2-ylidenemethyl]-3-methylbenzonitrile,
2-[5-benzyl-5-ethyl-1,4-dimethyl-3,6-dioxopiperazin-2-ylidenemethyl]-3-ethenylbenzonitrile,
2-[5-benzyl-5-ethyl-1-methyl-3,6-dioxopiperazin-2-ylidenemethyl]benzonitrile,
2-[5-benzyl-5-ethyl-1-methyl-3,6-dioxopiperazin-2-ylidenemethyl]-3-fluorobenzonitrile,
2-[5-benzyl-5-ethyl-1-methyl-3,6-dioxopiperazin-2-ylidenemethyl]-3-methoxybenzonitrile,
2-[5-benzyl-5-ethyl-1-methyl-3,6-dioxopiperazin-2-ylidenemethyl]-3,4-difluorobenzonitrile,
2-[5-benzyl-5-ethyl-1-methyl-3,6-dioxopiperazin-2-ylidenemethyl]-3-methylbenzonitrile,
2-[5-benzyl-5-ethyl-1-methyl-3,6-dioxopiperazin-2-ylidenemethyl]-3-ethenylbenzonitrile,
3-benzyl-6-[1-(2-nitrophenyl)methylidene]-1,3,4-trimethylpiperazine-2,5-dione,
3-benzyl-6-[1-(2-fluoro-6-nitrophenyl)methylidene]-1,3,4-trimethylpiperazine-2,5-dione,
3-benzyl-6-[1-(2,3-difluoro-6-nitrophenyl)methylidene]-1,3,4-trimethylpiperazine-2,5-dione,
3-benzyl-6-[1-(2-methoxy-6-nitrophenyl)methylidene]-1,3,4-trimethylpiperazine-2,5-dione,
3-benzyl-6-[1-(2-methyl-6-nitrophenyl)methylidene]-1,3,4-trimethylpiperazine-2,5-dione,
3-benzyl-6-[1-(2-ethenyl-6-nitrophenyl)methylidene]-1,3,4-trimethylpiperazine-2,5-dione,
3-benzyl-6-[1-(2-nitrophenyl)methylidene]-1,3-dimethylpiperazine-2,5-dione,
3-benzyl-6-[1-(2-fluoro-6-nitrophenyl)methylidene]-1,3-dimethylpiperazine-2,5-dione,
3-benzyl-6-[1-(2,3-difluoro-6-nitrophenyl)methylidene]-1,3-dimethylpiperazine-2,5-dione,
3-benzyl-6-[1-(2-methoxy-6-nitrophenyl)methylidene]-1,3-dimethylpiperazine-2,5-dione,
3-benzyl-6-[1-(2-methyl-6-nitrophenyl)methylidene]-1,3-dimethylpiperazine-2,5-dione,
3-benzyl-6-[1-(2-ethenyl-6-nitrophenyl)methylidene]-1,3-dimethylpiperazine-2,5-dione,
3-benzyl-6-[1-(2-nitrophenyl)methylidene]-3-ethyl-1,4-dimethylpiperazine-2,5-dione,
3-benzyl-6-[1-(2-fluoro-6-nitrophenyl)methylidene]-3-ethyl-1,4-dimethylpiperazine-2,5-dione,
3-benzyl-6-[1-(2,3-difluoro-6-nitrophenyl)methylidene]-3-ethyl-1,4-dimethylpiperazine-2,5-dione,
3-benzyl-6-[1-(2-methoxy-6-nitrophenyl)methylidene]-3-ethyl-1,4-dimethylpiperazine-2,5-dione,
3-benzyl-6-[1-(2-methyl-6-nitrophenyl)methylidene]-3-ethyl-1,4-dimethylpiperazine-2,5-dione,
3-benzyl-6-[1-(2-ethenyl-6-nitrophenyl)methylidene]-3-ethyl-1,4-dimethylpiperazine-2,5-dione,
3-benzyl-6-[1-(2-nitrophenyl)methylidene]-3-ethyl-1-methylpiperazine-2,5-dione,
3-benzyl-6-[1-(2-fluoro-6-nitrophenyl)methylidene]-3-ethyl-1-methylpiperazine-2,5-dione,
3-benzyl-6-[1-(2,3-difluoro-6-nitrophenyl)methylidene]-3-ethyl-1-methylpiperazine-2,5-dione,
3-benzyl-6-[1-(2-methoxy-6-nitrophenyl)methylidene]-3-ethyl-1-methylpiperazine-2,5-dione,
3-benzyl-6-[1-(2-methyl-6-nitrophenyl)methylidene]-3-ethyl-1-methylpiperazine-2,5-dione,
3-benzyl-6-[1-(2-ethenyl-6-nitrophenyl)methylidene]-3-ethyl-1-methylpiperazine-2,5-dione,
2-[5-(4-fluorobenzyl)-1,4,5-trimethyl-3,6-dioxopiperazin-2-ylidenemethyl]benzonitrile,
2-[5-(4-fluorobenzyl)-1,4,5-trimethyl-3,6-dioxopiperazin-2-ylidenemethyl]-3-fluorobenzonitrile,
2-[5-(4-fluorobenzyl)-1,4,5-trimethyl-3,6-dioxopiperazin-2-ylidenemethyl]-3-methoxybenzonitrile,
2-[5-(4-fluorobenzyl)-1,4,5-trimethyl-3,6-dioxopiperazin-2-ylidenemethyl]-3,4-difluorobenzonitrile,
2-[5-(4-fluorobenzyl)-1,4,5-trimethyl-3,6-dioxopiperazin-2-ylidenemethyl]-3-methylbenzonitrile,
2-[5-(4-fluorobenzyl)-1,4,5-trimethyl-3,6-dioxopiperazin-2-ylidenemethyl]-3-ethenylbenzonitrile,
2-[5-(4-fluorobenzyl)-1,5-dimethyl-3,6-dioxopiperazin-2-ylidenemethyl]-benzonitrile,
2-[5-(4-fluorobenzyl)-1,5-dimethyl-3,6-dioxopiperazin-2-ylidenemethyl]-3-fluorobenzonitrile,
2-[5-(4-fluorobenzyl)-1,5-dimethyl-3,6-dioxopiperazin-2-ylidenemethyl]-3-methoxybenzonitrile,
2-[5-(4-fluorobenzyl)-1,5-dimethyl-3,6-dioxopiperazin-2-ylidenemethyl]-3,4-difluorobenzonitrile,
2-[5-(4-fluorobenzyl)-1,5-dimethyl-3,6-dioxopiperazin-2-ylidenemethyl]-3-methylbenzonitrile,
2-[5-(4-fluorobenzyl)-1,5-dimethyl-3,6-dioxopiperazin-2-ylidenemethyl]-3-ethenylbenzonitrile,
2-[5-(4-fluorobenzyl)-5-ethyl-1,4-dimethyl-3,6-dioxopiperazin-2-ylidenemethyl]benzonitrile,
2-[5-(4-fluorobenzyl)-5-ethyl-1,4-dimethyl-3,6-dioxopiperazin-2-ylidenemethyl]-3-fluorobenzonitrile,
2-[5-(4-fluorobenzyl)-5-ethyl-1,4-dimethyl-3,6-dioxopiperazin-2-ylidenemethyl]-3-methoxybenzonitrile,
2-[5-(4-fluorobenzyl)-5-ethyl-1,4-dimethyl-3,6-dioxopiperazin-2-ylidenemethyl]-3,4-difluorobenzonitrile,
2-[5-(4-fluorobenzyl)-5-ethyl-1,4-dimethyl-3,6-dioxopiperazin-2-ylidenemethyl]-3-methylbenzonitrile,
2-[5-(4-fluorobenzyl)-5-ethyl-1,4-dimethyl-3,6-dioxopiperazin-2-ylidenemethyl]-3-ethenylbenzonitrile,
2-[5-(4-fluorobenzyl)-5-ethyl-1-methyl-3,6-dioxopiperazin-2-ylidenemethyl]benzonitrile,
2-[5-(4-fluorobenzyl)-5-ethyl-1-methyl-3,6-dioxopiperazin-2-ylidenemethyl]-3-fluorobenzonitrile,
2-[5-(4-fluorobenzyl)-5-ethyl-1-methyl-3,6-dioxopiperazin-2-ylidenemethyl]-3-methoxybenzonitrile,
2-[5-(4-fluorobenzyl)-5-ethyl-1-methyl-3,6-dioxopiperazin-2-ylidenemethyl]-3,4-difluorobenzonitrile,
2-[5-(4-fluorobenzyl)-5-ethyl-1-methyl-3,6-dioxopiperazin-2-ylidenemethyl]-3-methylbenzonitrile,
2-[5-(4-fluorobenzyl)-5-ethyl-1-methyl-3,6-dioxopiperazin-2-ylidenemethyl]-3-ethenylbenzonitrile,
3-(4-fluorobenzyl)-6-[1-(2-nitrophenyl)methylidene]-1,3,4-trimethylpiperazine-2,5-dione,
3-(4-fluorobenzyl)-6-[1-(2-fluoro-6-nitrophenyl)methylidene]-1,3,4-trimethylpiperazine-2,5-dione,
3-(4-fluorobenzyl)-6-[1-(2,3-difluoro-6-nitrophenyl)methylidene]-1,3,4-trimethylpiperazine-2,5-dione,
3-(4-fluorobenzyl)-6-[1-(2-methoxy-6-nitrophenyl)methylidene]-1,3,4-trimethylpiperazine-2,5-dione,
3-(4-fluorobenzyl)-6-[1-(2-methyl-6-nitrophenyl)methylidene]-1,3,4-trimethylpiperazine-2,5-dione,
3-(4-fluorobenzyl)-6-[1-(2-ethenyl-6-nitrophenyl)methylidene]-1,3,4-trimethylpiperazine-2,5-dione,
3-(4-fluorobenzyl)-6-[1-(2-nitrophenyl)methylidene]-1,3-dimethylpiperazine-2,5-dione,
3-(4-fluorobenzyl)-6-[1-(2-fluoro-6-nitrophenyl)methylidene]-1,3-dimethylpiperazine-2,5-dione,
3-(4-fluorobenzyl)-6-[1-(2,3-difluoro-6-nitrophenyl)methylidene]-1,3-dimethylpiperazine-2,5-dione,
3-(4-fluorobenzyl)-6-[1-(2-methoxy-6-nitrophenyl)methylidene]-1,3-dimethylpiperazine-2,5-dione,
3-(4-fluorobenzyl)-6-[1-(2-methyl-6-nitrophenyl)methylidene]-1,3-dimethylpiperazine-2,5-dione,
3-(4-fluorobenzyl)-6-[1-(2-ethenyl-6-nitrophenyl)methylidene]-1,3-dimethylpiperazine-2,5-dione,
3-(4-fluorobenzyl)-6-[1-(2-nitrophenyl)methylidene]-3-ethyl-1,4-dimethylpiperazine-2,5-dione,
3-(4-fluorobenzyl)-6-[1-(2-fluoro-6-nitrophenyl)methylidene]-3-ethyl-1,4-dimethylpiperazine-2,5-dione,
3-(4-fluorobenzyl)-6-[1-(2,3-difluoro-6-nitrophenyl)methylidene]-3-ethyl-1,4-dimethylpiperazine-2,5-dione,
3-(4-fluorobenzyl)-6-[1-(2-methoxy-6-nitrophenyl)methylidene]-3-ethyl-1,4-dimethylpiperazine-2,5-dione,
3-(4-fluorobenzyl)-6-[1-(2-methyl-6-nitrophenyl)methylidene]-3-ethyl-1,4-dimethylpiperazine-2,5-dione,
3-(4-fluorobenzyl)-6-[1-(2-ethenyl-6-nitrophenyl)methylidene]-3-ethyl-1,4-dimethylpiperazine-2,5-dione,
3-(4-fluorobenzyl)-6-[1-(2-nitrophenyl)methylidene]-3-ethyl-1-methylpiperazine-2,5-dione,
3-(4-fluorobenzyl)-6-[1-(2-fluoro-6-nitrophenyl)methylidene]-3-ethyl-1-methylpiperazine-2,5-dione,
3-(4-fluorobenzyl)-6-[1-(2,3-difluoro-6-nitrophenyl)methylidene]-3-ethyl-1-methylpiperazine-2,5-dione,
3-(4-fluorobenzyl)-6-[1-(2-methoxy-6-nitrophenyl)methylidene]-3-ethyl-1-methylpiperazine-2,5-dione,
3-(4-fluorobenzyl)-6-[1-(2-methyl-6-nitrophenyl)methylidene]-3-ethyl-1-methylpiperazine-2,5-dione, 3-(4-fluorobenzyl)-6-[1-(2-ethenyl-6-nitrophenyl)methylidene]-3-ethyl-1-methylpiperazine-2,5-dione,
2-[5-benzyl-1,4,5-trimethyl-3,6-dioxopiperazin-2-ylidenemethyl]-3-bromobenzonitrile,
2-[5-benzyl-1,4,5-trimethyl-3,6-dioxopiperazin-2-ylidenemethyl]-isophtalonitrile,
3-benzyl-6-[1-(2,3-difluoro-6-nitrophenyl)-methylidene]-1,3,4-trimethylpiperazin-2,5-dione,
3-benzyl-6-[1-(2-nitro-5-methoxyphenyl)-methylidene]-1,3,4-trimethylpiperazin-2,5-dione,
2-[5-benzyl-1,4,5-trimethyl-3,6-dioxopiperazin-2-ylidenemethyl]-3-nitrobenzonitrile,
3-benzyl-6-[1-(2-ethenyl-6-nitrophenyl)-methylidene]-1,3,4-trimethylpiperazin-2,5-dione,
3-benzyl-6-[1-(3-chloro-2-nitrophenyl)-methylidene]-1,3,4-trimethylpiperazin-2,5-dione,
3-benzyl-6-[1-(2-nitro-6-trifluoromethylphenyl)-methylidene]-1,3,4-trimethylpiperazin-2,5-dione,
2-[5-benzyl-1,5-dimethyl-3,6-dioxopiperazin-2-ylidenemethyl]benzonitrile,
3-benzyl-6-[1-(2-methoxy-6-nitrophenyl)-methylidene]-1,3,4-trimethylpiperazin-2,5-dione,
2-[5-benzyl-1,4,5-trimethyl-3,6-dioxopiperazin-2-ylidenemethyl]-4-fluorobenzonitrile,
2-[5-benzyl-1,4,5-trimethyl-3,6-dioxopiperazin-2-ylidenemethyl]-5-methylbenzonitrile,
2-[5-benzyl-1,4,5-trimethyl-3,6-dioxopiperazin-2-ylidenemethyl]-6-fluorobenzonitrile,
3-benzyl-1,3,4-trimethyl-6-[2-(1-methyl-1H-pyrrol-2-yl)-benzylidene]piperazin-2,5-dione,
3-benzyl-6-(2-furan-2-yl-benzylidene)-1,3,4-trimethylpiperazin-2,5-dione,
2-[5-benzyl-5-fluoromethyl-1,4-dimethyl-3,6-dioxopiperazin-2-ylidenemethyl]-benzonitrile,
3-benzyl-1,3,4-trimethyl-6-(4-methyl-2-nitrobenzylidene)-piperazin-2,5-dione,
2-[5-benzyl-1,4,5-trimethyl-3,6-dioxopiperazin-2-ylidenemethyl]-4-methoxybenzonitrile,
3-benzyl-6-[2-(2-chloropyrimidin-5-yl)-benzylidene]-1,3,4-trimethylpiperazin-2,5-dione,
3-benzyl-6-[2-(6-fluoropyridin-2-yl)-benzylidene]-1,3,4-trimethylpiperazin-2,5-dione,
2-[5-benzyl-1,4,5-trimethyl-3,6-dioxopiperazin-2-ylidenemethyl]-4-fluorobenzonitrile,
2-[5-benzyl-1,4,5-trimethyl-3,6-dioxopiperazin-2-ylidenmethyl]-4-trifluoromethylbenzonitril,
3-benzyl-1,3,4-trimethyl-6-[2-(1-methyl-1H-imidazol-2-yl)-benzylidene]-piperazin-2,5-dione,
3-benzyl-3-fluoromethyl-1,4-dimethyl-6-(2-nitrobenzylidene)-piperazin-2,5-dione,
3-benzyl-6-(5-bromo-2-nitrobenzylidene)-1,3,4-trimethylpiperazin-2,5-dione,
2-[5-benzyl-1,4,5-trimethyl-3,6-dioxopiperazin-2-ylidenemethyl]-4-difluoromethoxybenzonitrile,
2-[5-benzyl-1,4,5-trimethyl-3,6-dioxopiperazin-2-ylidenemethyl]-4-methansulfonylbenzonitrile,
2-[5-benzyl-1,4,5-trimethyl-3,6-dioxopiperazin-2-ylidenemethyl]-4-methansulfinylbenzonitrile,
2-[5-benzyl-1,4,5-trimethyl-3,6-dioxopiperazin-2-ylidenemethyl]-4-methylsulfanylbenzonitrile,
2-[5-benzyl-1,4,5-trimethyl-3,6-dioxopiperazin-2-ylidenemethyl]-3-fluoro-4-methoxybenzonitrile,
2-[5-benzyl-1,4,5-trimethyl-3,6-dioxopiperazin-2-ylidenemethyl]-4,6-difluorobenzonitrile,
3-benzyl-1,3,4-trimethyl-6-[2-(2-methyl-2H-pyrazol-3-yl)-benzylidene]piperazin-2,5-dione,
3-benzyl-1,3,4-trimethyl-6-[2-(5-methyl-thiophen-2-yl)-benzylidene]-piperazin-2,5-dione,
3-benzyl-1,3,4-trimethyl-6-[2-(3-methyl-thiophen-2-yl)-benzylidene]piperazin-2,5-dione,
2-[5-benzyl-4-ethyl-1,5-dimethyl-3,6-dioxopiperazin-2-ylidenemethyl]benzonitrile,
2-[5-benzyl-4-isopropyl-1,5-dimethyl-3,6-dioxopiperazin-2-ylidenemethyl]-benzonitrile,
2-[5-benzyl-4-butyl-1,5-dimethyl-3,6-dioxopiperazin-2-ylidenemethyl]-benzonitrile,
2-[4-allyl-5-benzyl-1,5-dimethyl-3,6-dioxopiperazin-2-ylidenemethyl]benzonitrile,
2-[5-benzyl-5-trifluoromethyl-1,4-dimethyl-3,6-dioxopiperazin-2-ylidenemethyl]benzonitrile,
3-benzyl-6-[1-(2-nitrophenyl)-methylidene]-1,4-dimethyl-3-trifluoromethylpiperazin-2,5-dione,
3-benzyl-6-[2-(6-chloropyridin-3-yl)-benzylidene]-1,3,4-trimethylpiperazin-2,5-dione,
2-[5-benzyl-1,5-dimethyl-4-prop-2-ynyl-3,6-dioxopiperazin-2-ylidenemethyl]-benzonitrile,
3-(3-fluorbenzyl)-6-[1-(2-nitrophenyl)-methylidene]-1,3,4-trimethylpiperazin-2,5-dione,
3-(3,5-difluorobenzyl)-6-[1-(2-nitrophenyl)-methylidene]-1,3,4-trimethylpiperazin-2,5-dione,
2-[5-(2,3-difluorobenzyl)-1,4,5-trimethyl-3,6-dioxopiperazin-2-ylidenemethyl]-benzonitrile,
2-[5-(2,5-difluorobenzyl)-1,4,5-trimethyl-3,6-dioxopiperazin-2-ylidenemethyl]-benzonitrile,
2-[5-(2,6-difluorobenzyl)-1,4,5-trimethyl-3,6-dioxopiperazin-2-ylidenemethyl]-benzonitrile,
2-[5-(2-difluoromethoxybenzyl)-1,4,5-trimethyl-3,6-dioxopiperazin-2-ylidenemethyl]-benzonitrile,
2-[5-(3-difluoromethoxybenzyl)-1,4,5-trimethyl-3,6-dioxopiperazin-2-ylidenemethyl]-benzonitrile,
2-[5-(3-trifluoromethylbenzyl)-1,4,5-trimethyl-3,6-dioxopiperazin-2-ylidenemethyl]-benzonitrile,
3-(3-fluorobenzyl)-6-[1-(2-nitrophenyl)-methylidene]-1,3,4-trimethylpiperazin-2,5-dione,
2-[5-(2-cyanobenzyl)-1,4,5-trimethyl-3,6-dioxopiperazin-2-ylidenemethyl]-benzonitrile,
2-[5-(3-cyanobenzyl)-1,4,5-trimethyl-3,6-dioxopiperazin-2-ylidenemethyl]benzonitrile,
2-[5-(3,5-difluorobenzyl)-1,4,5-trimethyl-3,6-dioxopiperazin-2-ylidenemethyl]-benzonitrile,
2-[5-(3-nitrobenzyl)-1,4,5-trimethyl-3,6-dioxopiperazin-2-ylidenemethyl]-benzonitrile,
2-[5-(4-fluoro-3-methylbenzyl)-1,4,5-trimethyl-3,6-dioxopiperazin-2-ylidenemethyl]-benzonitrile,
2-[5-(4-fluoro-3-methoxybenzyl)-1,4,5-trimethyl-3,6-dioxopiperazin-2-ylidenemethyl]-benzonitrile,
1-allyl-3-benzyl-3,4-dimethyl-6-[1-(2-nitrophenyl)-methylidene]-piperazin-2,5-dione and
3-benzyl-6-[1-(2-nitrophenyl)-methylidene]-1-prop-2-ynyl-3,4-dimethylpiperazin-2,5-dione.

From among the compounds mentioned here in an exemplary manner and their salts, preference is given to those compounds and salts in which the exo double bond at the piperazine ring has the (Z) configuration. Also preferred are mixtures of the (E) isomer with the (Z) isomer in which the Z isomer is present in excess, in particular isomer mixtures having an E/Z ratio of not more than 1:2, in particular not more than 1:5.

From among the compounds mentioned here in an exemplary manner and their salts, preference is given to those compounds and salts in which the carbon atoms which carries the radical R⁶ has the S configuration, and also to enantiomer mixtures having an enantiomeric excess with respect to the S enantiomer, in particular those having an ee (enantiomeric excess) of at least 70%, particularly preferably at least 80% and especially at least 90%. Preference is also given to the racemates of these compounds and their salts.

The compounds according to the invention can be prepared by standard processes of organic chemistry, for example a process (hereinbelow referred to as process A) which comprises the following steps:
i) provision of a compound of the general formula II

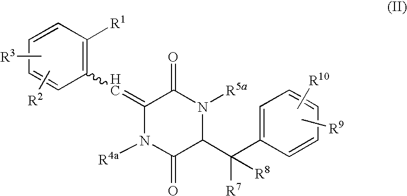

in which R¹, R², R³, R⁷, R⁸, R⁹ and R¹⁰ have the meanings mentioned above, in particular one of the meanings mentioned as being preferred, R^{4a} is hydrogen or a protective group or has one of the meanings given for R⁴ and R^{5a} has one of the meanings given for R⁵ or is a protective group;
ii) if appropriate reaction of the compound II in which R^{4a} is hydrogen with an alkylating agent of the formula R⁴—X¹ in which R⁴ has the meanings given above and X¹ is a nucleophilically displaceable leaving group, in the presence of a base;
iii) if appropriate reaction of the compound II in which R^{5a} is hydrogen with an alkylating agent of the formula R⁵—X¹ or an acylating agent of the formula R⁵—X² in which R⁵ has the meanings given above different from hydrogen and X¹ and X² are a nucleophilically displaceable leaving group, in the presence of a base;
iv) reaction of the compound II with an alkylating agent of the formula R⁶—X in which R⁶ has the meanings given above and X is a nucleophilically displaceable leaving group, in the presence of a base; and
v) if R^{4a} and/or R^{5a} are/is a protective group, removal of the protective group and, if appropriate, reaction of the resulting compound II in which R^{4a} and/or R^{5a} are/is hydrogen with an alkylating agent of the formula R⁴—X¹ and/or R⁵—X¹ or an acylating agent R⁵—X² in which R⁴ and/or R⁵ have/has the meanings given above different from hydrogen and X¹ and X² are a nucleophilically displaceable leaving group, in the presence of a base.

If the radical R^{4a} in formula II is hydrogen, the alkylation step ii) introduces the radical R⁴. If the radical R^{4a} in formula II is a protective group, this group is initially removed, which gives a compound in which R^{4a} is hydrogen into which the alkylation step ii) introduces the radical R⁴. If R^{5a} in formula II is hydrogen, the radical R⁵ can be introduced by an alkylation or acylation step iii). If R⁴ and R⁵ are identical, the steps ii) and iii) can be carried out simultaneously or successively in any order. If the radicals R⁴, R⁵ and R⁶ are identical, the step iv) can be carried out simultaneously with step(s) ii) and/or iii) or subsequently thereto.

The alkylation in step iv) and also the alkylation or acylation in steps ii) and iii) can be carried out analogously to standard processes of alkylation or acylation, for example according to the methods described by I. O. Donkor et al., Bioorg. Med. Chem. Lett. 11 (19) (2001), 2647-2649, B. B. Snider et al., Tetrahedron 57 (16) (2001), 3301-3307,1. Yasuhiro et al., Heterocycles, 45, 1997, 1151, J. Am. Chem. Soc. 105, 1983, 3214, J. Am. Chem. Soc. 124(47) (2002), 14017-14019, Chem. Commun. 1998, 659 or M. Falorni et al., Europ. J. Org. Chem. (8) (2000), 1669-1675.

To this end, in step iv) the piperazine compound of the formula II is reacted with a suitable alkylating agent, hereinbelow compound X—R⁶, which gives a piperazine compound of the formula I (see, for example, J. Am. Chem. Soc. 105, 1983, 3214).

In the alkylating agents X—R⁶, X can be halogen, in particular chlorine, bromine or iodine, or O—SO₂—R^{m} with R^{m} having the meaning C₁-C₄-alkyl or aryl, which are optionally substituted by halogen, C₁-C₄-alkyl or halo-C₁-C₄-alkyl.

The reaction is usually carried out at temperatures in the range from −78° C. to the boiling point of the reaction mixture, preferably from −50° C. to 65° C., particularly preferably from −30° C. to 65° C. In general, the reaction is carried out in a solvent, preferably in an inert organic solvent.

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butanol, water, dimethyl sulfoxide, N-methylpyrrolidone, dimethylformamide and dimethylacetamide, and also morpholine and N-methylmorpholine and mixtures thereof. Preferred solvents are toluene, dichloromethane, tetrahydrofuran, N-methylpyrrolidone, dimethylformamid and mixtures thereof.

In general, the alkylation of the compound II in step iv) is carried out using the alkylating agent R⁶—X in the presence of a base. Suitable bases are inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. The bases are generally employed in equimolar amounts. They can also be used in excess or even as solvent. In a preferred embodiment, the base is employed in an equimolar amount or an essentially equimolar amount. In a further preferred embodiment, the base used is sodium hydride.

The alkylation or acylation in the optional steps ii) and iii) can be carried out analogously to the methods given for step iv), for example according to the methods described in Heterocycles, 45, 1997, 1151, and Chem. Commun. 1998, 659. The optional alkylation or acylation in step v) can be carried out in the same manner.

To this end, in steps ii) and iii) the piperazine compound of the formula II where R^{4a}=hydrogen and/or R^{5a}=hydrogen is reacted with a suitable alkylating agent, hereinbelow compound X¹—R⁴ or X¹—R⁵, or an acylating agent, hereinbelow compound X²—R⁵, which gives a piperazine compound of the formula I where R⁵≠hydrogen.

In the alkylating agents X¹—R⁴ and X¹—R⁵, X¹ may be halogen or O—SO₂—R^{m} where R^{m} has the meaning C₁-C₄-alkyl or aryl which are optionally substituted by halogen, C₁-C₄-alkyl or halo-C₁-C₄-alkyl. In the alkylating agents X¹—R⁴ and X¹—R⁵, R⁴ and R⁵ independently of one another are C₁-C₄-alkyl, C₃-C₄-alkenyl or C₃-C₄-alkynyl. In the acylating agent R⁵—X², R⁵ is a radical C(O)R⁵¹ in which R⁵¹ has the meanings mentioned above. X² is generally halogen, for example chlorine, or a group O—C(O)—R⁵¹.

With respect to temperatures, bases and solvents, what was said for step iv) applies in an analogous manner.

If in formula II one or both radicals R^{4a} and R^{5a} is/are a protective group/protective groups, this/these protective group(s) is/are removed in step v). This gives a compound of the general formula I where R⁴ and R⁵═H, hereinbelow also referred to as compound I*. One or two new radical(s) R⁴ or/and R⁵ different from hydrogen is/are then introduced by alkylation or acylation analogously to steps ii) and iii) into the compound I*.

Suitable protective groups for the nitrogen atoms of the piperazine ring are in particular the radicals C(O)R⁵¹ mentioned above, for example the acetyl radical. The introduction of these protective groups can be carried out analogously to known processes of protective group chemistry, for example by reaction with anhydrides of the formula (R⁵¹C(O))₂O, for example according to the method described in Green, Wuts, Protective Groups in Organic Synthesis, 3rd ed. 1999, John Wiley and Sons, p. 553. The removal of a protective group R^{4a}, R^{5a} can be carried out analogously to known processes of protective group chemistry.

Besides, compounds of the formula II are known, for example from PCT/EP2007/050067 (=WO 2007/077247), the entire content of which is hereby included be way of reference.

The preparation of the compounds II is generally carried out by dehydrating the corresponding alcohol IIa,

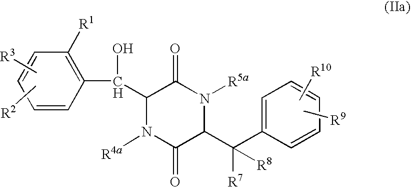

In formula IIa, R¹, R², R³, R^{4a}, R^{5a}, R⁷, R⁸, R⁹ and R¹⁰ have the meanings mentioned above, in particular one of the meanings mentioned as being preferred. In a first variant (variant A.1), the alcohol function of the compound IIa can initially be converted into a suitable leaving group, and this can then be eliminated formally as compound H-LG. The elimination reaction is preferably carried out in the presence of a suitable base.

The leaving group LG is a customary leaving group easy to prepare from a hydroxyl group. Examples of these are 4-toluenesulfonyloxy (LG=-O—SO₂C₆H₄CH₃), trifluoromethanesulfonyloxy (LG=-O—SO₂CF₃) and methanesulfonyloxy (LG=-O—SO₂CH₃), the latter being particularly suitable. Such a leaving group is introduced according to customary processes, for example by reacting the alcohol IIa with a base and then with the appropriate sulfonyl chloride, for example with methanesulfonyl chloride or trifluoromethanesulfonyl chloride. Suitable bases are the bases listed below for the elimination. However, preference is given to using bases which are soluble in organic solvents, for example the amines or nitrogen heterocycles mentioned below. In particular, use is made of pyridine or substituted pyridines, such as dimethylaminopyridine, lutidine or collidine, or mixtures thereof. Expediently, the organic bases are chosen such that they also act as solvent.

Bases suitable for the elimination are, in general inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. It is, of course, also possible to use a mixture of different bases.

Particularly suitable are, however, bases which are sufficiently basic, but essentially not nucleophilic, for example sterically hindered alkali metal alkoxides, for example alkali metal tert-butoxides, such as potassium tert-butoxide, and in particular cyclic amidines, such as DBU (1,8-diazabicyclo[5.4.0]undec-7-ene) and DBN (1,5-diazabicyclo[3.4.0]-non-5-ene). Preference is given to using the amidines mentioned last.

The elimination is generally carried out in a solvent, preferably in an inert organic solvent. Suitable inert organic solvents include aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butanol, water, and also dimethyl sulfoxide, dimethylformamide and dimethylacetamide, and also morpholine and N-methylmorpholine. It is also possible to use mixtures of the solvents mentioned.

Preference is given to using tetrahydrofuran.

The dehydration of alcohols IIa by conversion of the alcohol function into a good leaving group and subsequent elimination can be carried out analogously to known processes of the prior art, for example analogously to the processes described in Helv. Chim. Acta 1947, 30, 1454; Liebigs Ann. Chem. 1992, (7), 687-692, Carbanions. 24. Rearrangements of (E)- and (Z)-2,2-diphenyl-3-pentenylalkali metal compounds; Sch. Chem., Georgia Inst. Technol., Atlanta, Ga., USA; J. Org. Chem. 1989, 54(7), 1671-1679; Chemical & Pharmaceutical Bulletin 1986, 34(7), 2786-2798, the entire contents of which are included herein by way of reference.

In a second variant (variant A.2), the preparation of the compound II by dehydration of the compound IIa is carried out in the presence of a suitable dehydrating agent.

Suitable dehydrating agents are, for example, the system triphenylphosphine/DEAD (DEAD=diethyl azodicarboxylate) and Burgess reagent. In general, the combination of triphenylphosphine and DEAD is employed for the targeted inversion at a hydroxyl-substituted center of chirality (Mitsunobu reaction); however, in the presence of nucleophiles it acts as a mild dehydrating agent. With respect to the compound IIa, the system is preferably employed in excess, where the two components triphenylphosphine and DEAD are suitably present in an approximately equimolar ratio.

Burgess reagent is the zwitterion methyl N-(triethylammoniumsulfonylcarbamate ((C₂H₅)₃N⁺—SO₂—N⁻—COOCH₃), a mild dehydrating agent. With respect to the alcohol II, this can be employed in equimolar amounts or in a molar excess. The reaction with Burgess reagent is usually carried out in an inert organic solvent. Suitable inert organic solvents include aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, and ketones, such as acetone, methyl ethyl ketone, diethyl ketone and tert-butyl methyl ketone. Preference is given to using aromatic hydrocarbons or mixtures thereof and especially toluene.

The dehydration of alcohols IIa with dehydrating agents can be carried out analogously to known processes of the prior art, for example analogously to the processes described in Synthesis 2003, 201 and J. Indian Sci. 2001, 81, 461, the entire contents of which are included herein by way of reference.

The alcohols of the formula IIa can be prepared, for example, analogously to processes known from the literature by cyclization of corresponding dipeptide precursors, for example analogously to the method described by T. Kawasaki et al., Org. Lett. 2(19) (2000), 3027-3029, Igor L. Rodionov et al., Tetrahedron 58(42) (2002), 8515-8523 or A. L. Johnson et al., Tetrahedron 60 (2004), 961-965.

The alcohols of the formula IIa can also be prepared by coupling, in an aldol reaction, a benzaldehyde of the formula III with a piperazine compound IV, as illustrated in the scheme below:

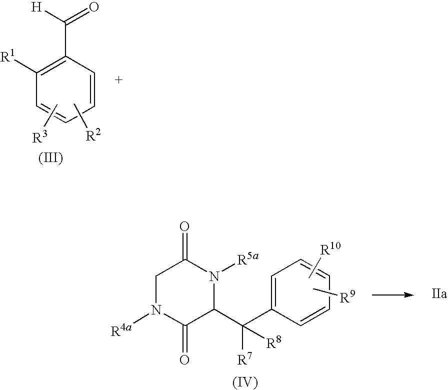

In the formulae III and IV, the variables R¹, R², R³, R^{4a}, R^{5a}, R⁷, R⁸, R⁹ and R¹⁰ have the meanings given for formula II.

The reaction of III with IV in the sense of an aldol reaction is generally carried out in the presence of suitable bases. Suitable bases are those which are usually employed for aldol reactions. Suitable reaction conditions are known from the prior art and are described, for example, in J. Org. Chem. 2000, 65 (24), 8402-8405, the entire content of which is hereby included by way of reference.

The reaction of the compound III with the compound IV can also afford the corresponding aldol condensation product, i.e. compounds of the formula II, directly. This is the case in particular when in the compound IV the radicals R^{4a} and R^{5a} are acyl groups, for example a group of the formula R⁵²C(O)— in which R⁵² has one of the meanings given for R⁵¹ and is in particular C₁-C₄-alkyl, for example methyl.

Such aldol condensations can be carried out analogously to the processes described in J. Org. Chem. 2000, 65 (24), 8402-8405, Synlett 2006, 677 and J. Heterocycl. Chem. 1988, 25, 591, the entire contents of which are hereby included by way of reference.

The aldol condensation is typically carried out in the presence of suitable bases. Suitable bases are those which are usually employed for aldol condensations. Preference is given to using an alkali metal or alkaline earth metal carbonate as base, for example sodium carbonate, potassium carbonat or cesium carbonate or mixtures thereof.

The reaction is preferably carried out in an inert, preferably aprotic organic solvent. Examples of suitable solvents are in particular dichloromethane, dichloroethane, chlorbenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, dimethylformamide, N-methylpyrrolidone and dimethylacetamide. Preferred solvents are in particular selected from the group consisting of dimethylformamide, N-methylpyrrolidone and dimethylacetamide.

The temperatures required for the aldol condensation are generally in the range of from 0° C. to the boiling point of the solvent used and in particular in the range of from 10 to 80° C.

For the reaction of III with IV, it has been found to be advantageous for the radicals R^{4a} and R^{5a} in the compound IV to represent an acyl group, for example a group of the formula R⁵²C(O)—. The introduction of these protective groups into the compound IV can be carried out analogously to known processes of protective group chemistry, for example by reacting the corresponding NH-free compound (the compound of the formula IV where R^{4a}, R^{5a}=H) with anhydrides of the formula (R⁵²C(O))₂O, for example according to the method described in Green, Wuts, Protective Groups in Organic Synthesis, 3rd ed. 1999, John Wiley and Sons, p. 553. The removal of a protective group R^{4a}, R^{5a} can be carried out analogously to known processes of protective group chemistry.

If the radicals R^{4a} and R^{5a} in the compound IV represent an acyl group, these radicals are preferably removed after the aldol condensation, which gives a compound of the formula II where R^{4a}=R^{5a}=hydrogen. The radicals R^{4a} and R^{5a} are generally removed by hydrolysis, the radical R^{4a} frequently already being cleaved off under the conditions of an aldol condensation. Into the resulting compound II where R^{4a}=R^{5a}=hydrogen, the radical R⁴ and, if appropriate, the radical R⁵ are then introduced according to steps ii) and iii).

In a manner analogously to the method described here, it is also possible to prepare compounds of the formula I′:

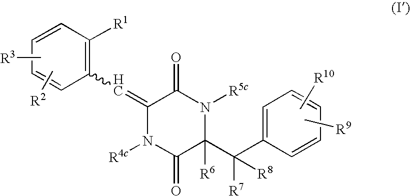

in which R¹, R², R³, R⁶, R⁷, R⁸, R⁹ and R¹⁰ have the meanings mentioned above, in particular one of the meanings mentioned as being preferred, R^{4c} is hydrogen or a protective group and R^{5c} has one of the meanings given for R⁵ or is a protective group. Preferred protective groups are the acyl groups mentioned above of the formula R⁵²C(O)— in which R⁵² has one of the meanings given for R⁵¹ and is in particular C₁-C₄-alkyl, for example methyl.

If R^{4c} and/or R^{5c} in formula I′ are protective groups/is a protective group, the protective groups R^{4c} and/or R^{5c} will be removed. This gives a compound I′ in which R^{4c} and, if appropriate, R^{5c} is hydrogen.

This compound I′ in which R^{4c} is hydrogen is then reacted with an alkylating agent of the formula R⁴—X¹, preferably in the presence of a base. If R^{5c} is hydrogen, the compound I′ is reacted with an alkylating agent of the formula R⁵—X¹ or an acylating agent of the formula R⁵—X², preferably in the presence of a base. For the reaction of compound I′ with the alkylating agents X¹—R^{4a}, X¹—R⁵ or X²—R⁵, what was said above for steps ii) and iii) applies analogously.

The compound I′ can be prepared analogously to the preparation of compound II by aldol addition of compound III with a compound IVa with subsequent elimination of water or, preferably, by reacting III with a compound IVa under the conditions of an aldol condensation:

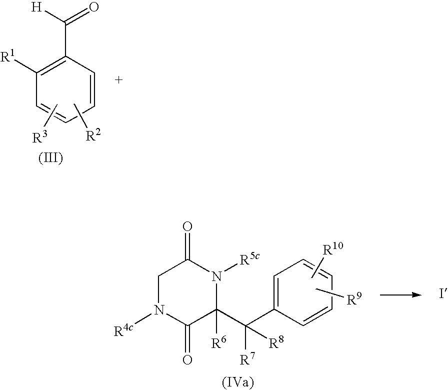

In this scheme, the variables R¹, R², R³, R^{4c}, R⁸, R⁶, R⁷, R⁸, R⁹ and R¹⁰ have the meanings mentioned above.

The aldehyde III is either commercially available or can be synthesized according to known processes for preparing aldehydes.

The compounds of the formulae IV and IVa can be prepared by intramolecular cyclization of compounds of the general formula V and Va, respectively, analogously to other processes known from the literature, for example according to T. Kawasaki et al., Org. Lett. 2(19) (2000), 3027-3029, Igor L. Rodionov et al., Tetrahedron 58(42) (2002), 8515-8523 or A. L. Johnson et al., Tetrahedron 60 (2004), 961-965.

If appropriate, the cyclization is followed by the introduction of a group R^{4a} or R^{4c}, R^{5a} or R^{5b} different from hydrogen if R^{4a} or R^{4c} and/or R^{5b} in the formulae V and Va is hydrogen.

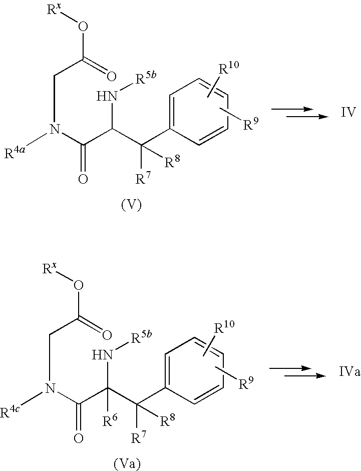

In formula V, the variables R^{4a}, R⁷, R⁸, R⁹ and R¹⁰ have the meanings mentioned above. R^{5b} is hydrogen, C₁-C₄-alkyl, C₃-C₄-alkenyl or C₃-C₄-alkynyl. R^{x} is here, for example, C₁-C₆-alkyl, in particular methyl or ethyl, or phenyl-C₁-C₆-alkyl, for example benzyl. In formula Va, the variables R^{4c}, R⁷, R⁸, R⁹ and R¹⁰ have the meanings mentioned above. R^{5b} is hydrogen, C₁-C₄-alkyl, C₃-C₄-alkenyl or C₃-C₄-alkynyl. R^{x} is here, for example, C₁-C₆-alkyl, in particular methyl or ethyl, or phenyl-C₁-C₆-alkyl, for example benzyl.

The cyclization of the compounds of the formula V or Va can be carried out in the presence of a base. In this case, the reaction is generally carried out at temperatures in the range of from 0° C. to the boiling point of the reaction mixture, preferably from 10° C. to 50° C., particularly preferably from 15° C. to 35° C. The reaction can be carried out in a solvent, preferably in an inert organic solvent.

Suitable inert organic solvents include aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, 2-butanol, isobutanol, tert-butanol, water, and also dimethyl sulfoxide, dimethylformamide and dimethylacetamide, and also morpholine and N-methylmorpholine. It is also possible to use mixtures of the solvents mentioned. The preferred solvent is a tetrahydrofuran/water mixture having a mixing ratio of from 1:10 to 10:1.

Suitable bases are, for example, inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. It is, of course, also possible to use a mixture of different bases. Preference is given in particular to potassium tert-butoxide, 2-hydroxypyridine or an aqueous solution of ammonia or a mixture of these bases. Preferably, only one of these bases is used. In a particularly preferred embodiment, the reaction is carried out in the presence of an aqueous solution of ammonia which may, for example, be of a strength of from 10 to 50% w/v. In another particularly preferred embodiment, the cyclization is carried out in a mixture comprising n-butanol or a mixture of butanol isomers (for example, a mixture of n-butanol and 2-butanol and/or isobutanol) and N-methylmorpholine, preferably under reflux conditions.

The cyclization of V or Va can also be carried out with acid catalysis, in the presence of activating compounds or thermally. The reaction of V in the presence of an acid is usually carried out at temperatures in the range of from 10° C. to the boiling point of the reaction mixture, preferably from 50° C. to the boiling point, particularly preferably at the boiling point under reflux. In general, the reaction is carried out in a solvent, preferably in an inert organic solvent.

Suitable solvents are, in principle, those which can also be used for the basic cyclization, in particular alcohols. In a preferred embodiment, the reaction is carried out in n-butanol or a mixture of different butanol isomers (for example a mixture of n-butanol and 2-butanol and/or isobutanol).

Suitable acids for the cyclization of V or Va are, in principle, both Brönstedt and Lewis acids. Use may be made in particular of inorganic acids, for example hydrohalic acids, such as hydrofluoric acid, hydrochloric acid, hydrobromic acid, inorganic oxoacids, such as sulfuric acid and perchloric acid, furthermore of inorganic Lewis acids, such as borin trifluoride, aluminum trichloride, iron(III) chloride, tin(IV) chloride, titanium(IV) chloride and zinc(II) chloride, and also of organic acids, for example carboxylic acids and hydroxycarboxylic acids, such as formic acid, acetic acid, propionic acid, oxalic acid, citric acid and trifluoroacetic acid, and also organic sulfonic acids, such as toluenesulfonic acid, benzenesulfonic acid, camphorsulfonic acid and the like. Of course, it is also possible to use a mixture of different acids.

In one embodiment of the process according to the invention, the reaction is carried out in the presence of organic acids, for example in the presence of carboxylic acids, such as formic acid, acetic acid or trifluoroacetic acid or a mixture of these acids. Preferably, only one of these acids is used. In a preferred embodiment, the reaction is carried out in acetic acid.

In a particularly preferred embodiment, the acidic cyclization is carried out in a mixture comprising n-butanol or a butanol isomer mixture (for example a mixture of n-butanol and 2-butanol and/or isobutanol), N-methylmorpholine and acetic acid, preferably under reflux conditions.

In a further embodiment of the invention, the conversion of V or Va is carried out by treatment with an activating agent in the presence of a base. In this case, R^{x} is hydrogen. An example of a suitable activating agent is di-(N-succinimidinyl) carbonate. Suitable activating agents are furthermore polystyrene- or non-polystyrene-supported di-cyclohexylcarbodiimide (DCC), diisopropylcarbodiimide, 1-ethyl-3-(dimethylaminopropyl)carbodiimide (EDAC), carbonyldiimidazole (CDI), chloroformic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCI) or sulfonyl chlorides, such as methanesulfonyl chloride, toluenesulfonyl chloride or benzenesulfonyl chloride. Suitable bases are the compounds cited for the basic cyclization. In one embodiment, the base used is triethylamine or N-ethyldiisopropylamine or mixtures thereof, particularly preferably N-ethyldiisopropylamine.

In a further embodiment of the invention, the conversion of V or Va is carried out exclusively by heating the reaction mixture (thermal cyclization). Here, the reaction is usually carried out at temperatures in the range of from 10° C. to the boiling point of the reaction mixture, preferably from 50° C. to the boiling point of the reaction mixture, particularly preferably at the boiling point of the reaction mixture under reflux. The reaction is generally carried out in a solvent, preferably in an inert organic solvent.

In principle, suitable solvents are those solvents which can be used for the basic cyclization. Preference is given to polar aprotic solvents, for example dimethyl sulfoxide or dimethylformamide or mixtures thereof. In a preferred embodiment, the reaction is carried out in dimethyl sulfoxide.

If in compound V or Va one or both radicals R^{4a} or R^{4c} and/or R^{5b} is/are hydrogen, the piperazine nitrogens can then, to introduce the radicals R^{4a} or R^{4c} and/or R^{5a} or R^{5c}, be alkylated using an acylating agent R^{4a}—X¹, R^{5a}—X¹, R^{4c}—X¹ or R^{5c}—X¹ or be provided with a protective group by reaction with an acylating agent R^{4a}—X², R^{5a}—X², R^{4c}—X² or R^{5c}—X². Here, R^{4a}, R^{4c}, R^{5a}, R^{5c}, X¹ and X² have the meanings given above.

For their part, the compounds of the formula V or Va can be prepared by the scheme shown below analogously to processes from the literature, for example according to Wilford L. Mendelson et al., Int. J. Peptide & Protein Research 35(3), (1990), 249-57, Glenn L. Stahl et al., J. Org. Chem. 43(11), (1978), 2285-6 or A. K. Ghosh et al., Org. Lett. 3(4), (2001), 635-638.

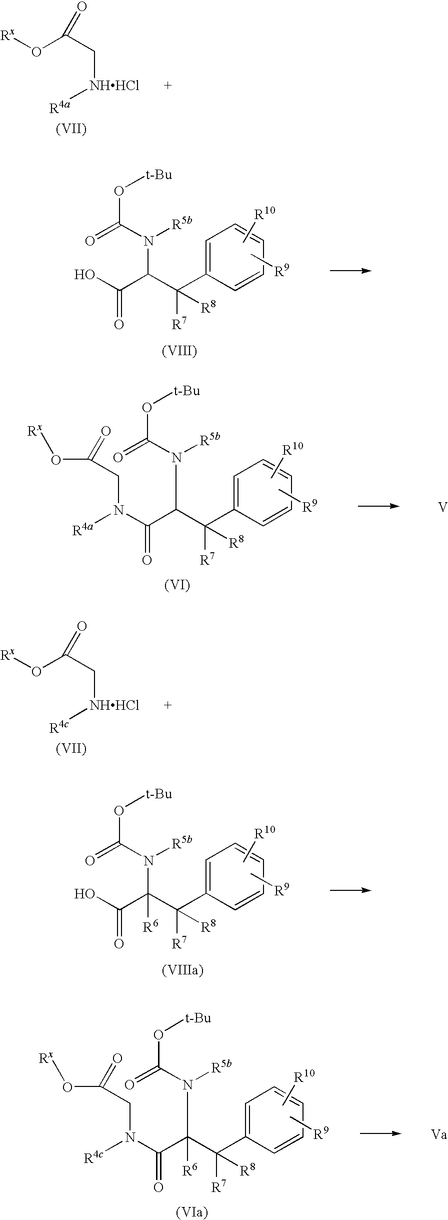

In the scheme, the variables R^{x}, R^{4a}, R^{4c}, R^{5b}, R⁶, R⁷, R⁸, R⁹ and R¹⁰ have the meanings given for formula V. The synthesis comprises, in a first step, the coupling of glycine ester compounds of the formula VII with Boc-protected phenylalanine compounds VIII or VIIIa in the presence of an activating agent. Instead of Boc, it is also possible to use another amino-protective group.

The reaction of a compound of the formula VII with a compound of the formula VIII or VIIIa is usually carried out at temperatures in the range of from −30° C. to the boiling point of the reaction mixture, preferably of from 0° C. to 50° C., particularly preferably of from 20° C. to 35° C. The reaction can be carried out in a solvent, preferably in an inert organic solvent.

In general, the reaction requires the presence of an activating agent. Suitable activating agents are condensing agents, such as, for example, polystyrene- or non-polystyrene-supported dicyclohexylcarbodiimide (DCC), diisopropylcarbodiimide, carbonyldiimidazole (CDI), 1-ethyl-3-(dimethylaminopropyl)carbodiimide (EDAC), chloroformic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)phosphoryl chloride (BOPCI) or sulfonyl chlorides, such as methane-sulfonyl chloride, toluenesulfonyl chloride or benzenesulfonyl chloride. According to one embodiment, a preferred activating agent is EDAC or DCC.

The reaction of VII with VIII or VIIIa is preferably carried out in the presence of a base. Suitable bases are the compounds listed for the cyclization of the dipeptide V to the piperazine IV. In one embodiment, the base used is triethylamine or N-ethyldiisopropylamine or a mixture thereof, particularly preferably N-ethyl-diisopropylamine.

The deprotection of the compound VI or VIa to give the compound V or Va can be carried out by customary processes, such as, for example, according to Glenn L. Stahl et al., J. Org. Chem. 43(11), (1978), 2285-6 or A. K. Ghosh et al., Org. Lett. 3(4), (2001), 635-638. The deprotection is typically carried out by treatment with an acid. Suitable acids are both Brönstedt acids and Lewis acids, preferably organic carboxylic acids, for example formic acid, acetic acid or trifluoroacetic acid or mixtures thereof. In a preferred embodiment, the reaction is carried out in the presence of trifluoroacetic acid.

The reaction is usually carried out at temperatures in the range of from −30° C. to the boiling point of the reaction mixture, preferably from 0° C. to 50° C., particularly preferably from 20° C. to 35° C. The reaction can be carried out in a solvent, preferably in an inert organic solvent.

Suitable solvents are, in principle, the solvents mentioned above in connection with the basic cyclization of V to IV, in particular tetrahydrofuran or dichloromethane or mixtures thereof. In a preferred embodiment, the reaction is carried out in dichloromethane.

If another protective group is used instead of Boc, the deprotection method used will, of course, be suitable for the protective group in question.

If the groups R^{4a} and R^{5a} or R^{4c} and R^{5c} in the compounds IV and IVa are hydrogen, the compounds IV and IVa can also be prepared by intermolecular cyclization of a glycine ester derivative VIIla with a phenylalanine compound VIIIb or VIIIc according to the schemes below:

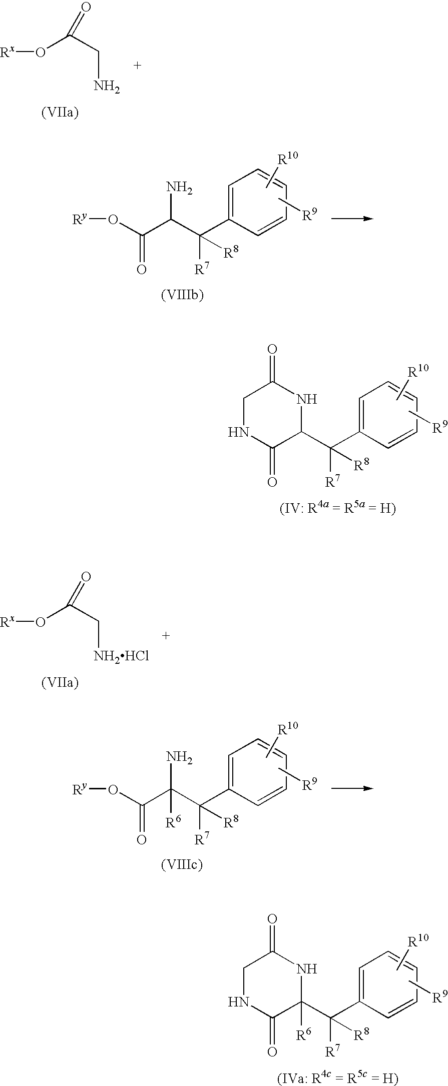

In the schemes, R^{x}, R⁶, R⁷, R⁸, R⁹ and R¹⁰ have the meanings given above. R^{y} is alkyl, for example methyl or ethyl. The intermolecular cyclization can be effected, for example, by a base, for example ammonia. The compounds VIIa and/or VIIIb or VIIIc can also be employed in the form of their acid addition salts, for example as hydrochlorides.

According to another embodiment (hereinbelow referred to as process B), the preparation of the compounds I comprises
i) providing a compound of the general formula IX

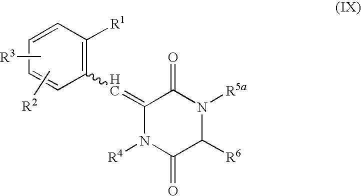

in which R¹, R², R³, R⁴ and R⁶ have the meanings mentioned above and R^{5a} has one of the meanings given for R⁵ different from hydrogen or is a protective group;
ii) reacting the compound IX with the benzyl compound of the formula X

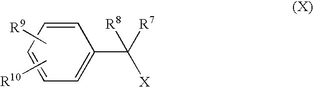

in which R⁷, R⁶, R⁹ and R¹⁰ have the meanings given above and X is a nucleophilically displaceable leaving group, in the presence of a base; and
iii) if R^{5a} is a protective group, removing the protective group.

In formula IX, R^{5a} has preferably one of the meanings given for R⁵ different from hydrogen. In formula X, the variable X has preferably one of the following meanings: halogen, in particular chlorine, bromine or iodine, or O—SO₂—R^{m} where R^{m} has the meaning of C₁-C₄-alkyl or aryl which are optionally substituted by halogen, C₁-C₄-alkyl or halo-C₁-C₄-alkyl. Suitable protective groups for the nitrogen atoms of the piperazine rings in IX are in particular the radicals C(O)R⁵¹ mentioned above, for example the acetyl radical.

The reaction of the compound IX with the compound X in step ii) can be carried out analogously to the method described in process A, step iv) or, for example, according to the method described in J. Am. Chem. Soc. 105, 1983, 3214. In a preferred embodiment, the reaction is carried out in the presence of sodium hydride as base in N-methylpyrrolidone as solvent.

The compounds IX can be provided, for example, by reacting the compound XI with a benzaldehyde compound XII, as illustrated in the scheme below.

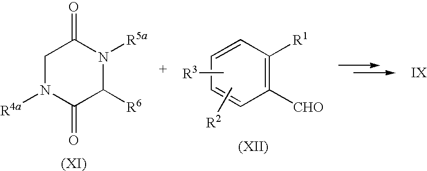

Here, R¹, R², R³, R^{5a} and R⁶ have the meanings mentioned above. R^{4a} has one of the meanings given above or is a protective group. Suitable protective groups for the nitrogen atoms of the piperazine ring in XI are in particular the radicals C(O)R⁵¹ mentioned above, for example the acetyl radical. R^{4a} and R^{5a} are in particular one of the radicals C(O)R⁵² mentioned above, for example acetyl radicals.

The reaction of XI with XII can be carried out under the conditions of an aldol condensation, as already described for the reaction of III with IV or IVa. Such aldol condensations can be carried out analogously to the processes described in J. Org. Chem. 2000, 65 (24), 8402-8405, Synlett 2006, 677, J. Heterocycl. Chem. 1988, 25, 591, which are hereby incorporated herein in their entirety.

The reaction is generally carried out in the presence of a base. The base used is preferably an alkali metal or alkaline earth metal carbonate, for example sodium carbonate, potassium carbonate or cesium carbonate, or mixtures thereof.

The reaction is preferably carried out in an inert, preferably aprotic organic solvent. Examples of suitable solvents are in particular dichloromethane, dichloroethane, chlorobenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, dimethylformamide, N-methylpyrrolidone and dimethylacetamide.

The compounds reacted are preferably those compounds XI in which R^{4a} and R^{5a} are a protective group and in particular an acyl radical R⁵²C(O)— (R⁵²=C₁-C₄-alkyl), for example an acetyl radical. Accordingly, the condensation reaction is generally followed by a removal of the protective groups. The removal of a protective group R^{4a}, R^{5a} can be carried out analogously to known processes of protective group chemistry, for example by the method described in Green, Wuts, Protective Groups in Organic Synthesis, 3rd ed. 1999, John Wiley and Sons, p. 553. A subsequent alkylation for introducing the radicals R⁴ and/or R⁵ can be carried out by the method given above in process A for the steps ii) and iii).

The compounds XI are known. Their preparation can be carried out analogously to the preparation of the compounds V described above, according to the scheme shown below:

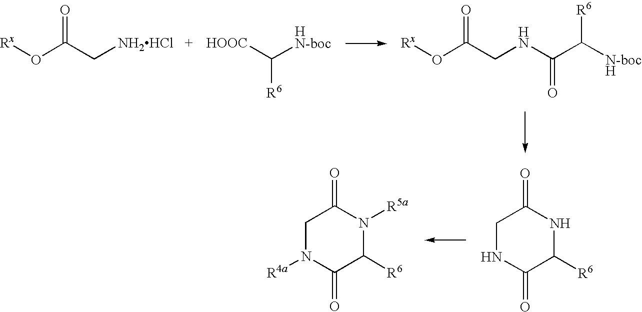

In this scheme, R^{4a}, R^{5a} and R⁶ have the meanings mentioned above. R^{x} is preferably C₁-C₄-alkyl or benzyl. Boc is a tert-butoxycarbonyl radical.

With respect to further details for the first reaction step, reference is made to the reaction of compound VII with the compound VIII or VIIIa or to the reaction of VIIa with VIIIb or VIIIc. The subsequent removal of the Boc protective group can be carried out analogously to the conversion of the compound VI into the compound V. The cyclization of the resulting deprotected compound can be carried out using the methods mentioned for the cyclization of the compound V. If R^{4a} and R^{5a} are a protective group, for example a radical C(O)R⁵¹, these protective groups can be introduced analogously to known processes of protective group chemistry, for example by reaction with anhydrides of the formula (R⁵¹C(O))₂O, for example by the method described in Green, Wuts, Protective Groups in Organic Synthesis, 3rd ed. 1999, John Wiley and Sons, p. 553.

The compounds of the formula I where R⁵≠H can also be prepared by reacting a piperazine compound of the formula I in which R⁵ is hydrogen with an alkylating agent or acylating agent which contains the radical R⁵ different from hydrogen. Such reactions can be carried out analogously to the methods discussed in connection with process A steps ii), iii) and v). Corresponding alkylations can also be carried out at the stages of the compounds VII, VIIa, VIII, VIIIa, VIIIb and VIIIc.

To this end, the piperazine compound of the formula I where R⁵=hydrogen is reacted with a suitable alkylating agent, hereinbelow compound X¹—R⁵, or acylating agent, hereinbelow compound X²—R⁵, which gives a piperazine compound of the formula I where R⁵≠hydrogen.

In the alkylating agents X¹—R⁵, X¹, can be halogen or O—SO₂—R^{m} where R^{m} has the meaning C₁-C₄-alkyl or aryl which are optionally substituted by halogen, C₁-C₄-alkyl or halo-C₁-C₄-alkyl. In acylating agents X²—R⁵, X² can be halogen, in particular Cl. Here, R⁵ is a radical (CO)R⁵¹.

The reaction is usually carried out at temperatures in the range of from −78° C. to the boiling point of the reaction mixture, preferably from −50° C. to 65° C., particularly preferably from −30° C. to 65° C. In general, the reaction is carried out in a solvent, preferably in an inert organic solvent.

Suitable solvents are the compounds mentioned for the cyclization of the dipeptide V to the piperazine IV, inter alia toluene, dichloromethane, tetrahydrofuran or dimethylformamide or mixtures thereof.

In a preferred embodiment, the compound I where R⁵═H is reacted with the alkylating or acylating agent in the presence of a base. Suitable bases are the compounds mentioned for the cyclization of the dipeptide V to the piperazine IV. The bases are generally employed in equimolar amounts. They can also be used in excess or even as solvent. In a preferred embodiment, the base is added in an equimolar amount or in an essentially equimolar amount. In a further preferred embodiment, the base used is sodium hydride.

Alternatively, the alkylation or acylation of the group NR⁵ in which R⁵ is H can also be carried out using the precursors. Thus, for example, compounds II, IV, V, VI, VIII in which R^{5a} or R^{5b} is H can be N-alkylated or N-acylated as described above. In the same manner, it is possible to alkylate the precursors II, IV, V, VI, VII in which the radical referred to as R⁴ or R^{4a} is hydrogen.

The compounds of the formula I can furthermore be modified at group R¹. Thus, for example, compounds of the formula I in which R¹ is CN, optionally substituted phenyl or an optionally substituted heterocyclic radical can be prepared from compounds I in which R¹ is halogen, such as chlorine, bromine or iodine, by conversion of the substituent R¹, for example analogously to the methods described by J. Tsuji, Top. Organomet. Chem. (14) (2005), 332 pp., J. Tsuji, Organic Synthesis with Palladium Compounds, (1980), 207 pp., Tetrahedron Lett. 42, 2001, p. 7473 or Org. Lett. 5, 2003, 1785.

To this end, a piperazine compound of the formula I which, as substituent R¹, has a halogen atom, such as chlorine, bromine or iodine, can be converted by reaction with a coupling partner which contains a group R¹ (compound R¹—X³) into another piperazine derivative of the formula I. In an analogous manner, it is also possible to react the compounds Ia, II and IIa.

The reaction is usually carried out in the presence of a catalyst, preferably in the presence of a transition metal catalyst. In general, the reaction is carried out in the presence of a base.

Suitable coupling reagents X³—R¹ are in particular those compounds in which X³, if R¹ is phenyl or a heterocyclic radical (heterocyclyl), denotes one of the following groups:
Zn—R^{l} where R^{l} is halogen, phenyl or heterocyclyl;
B(OR^{m})₂, where R^{m} is H or C₁-C₆-alkyl, where two alkyl substituents together may form a C₂-C₄-alkylene chain; or
SnRⁿ₃ where Rⁿ is C₁-C₆-alkyl.

This reaction is usually carried out at temperatures in the range from −78° C. to the boiling point of the reaction mixture, preferably from −30° C. to 65° C., particularly preferably at temperatures from 30° C. to 65° C. In general, the reaction is carried out in an inert organic solvent in the presence of a base.

Suitable solvents are the compounds mentioned in connection with the cyclization of the dipeptide IV to the piperazine V. In one embodiment of the process according to the invention, use is made of tetrahydrofuran with a catalytic amount of water; in another embodiment, only tetrahydrofuran is used.

Suitable bases are the compounds mentioned for the cyclization of the dipeptide IV to the piperazine V.

The bases are generally employed in equimolar amounts. They can also be employed in excess or even as solvent.

In a preferred embodiment of the process according to the invention, the base is added in an equimolar amount. In a further preferred embodiment, the base used is triethylamine or cesium carbonate, particularly preferably cesium carbonate.

Suitable catalysts for the process according to the invention are, in principle, compounds of the transition metals Ni, Fe, Pd, Pt, Zr or Cu. It is possible to use organic or inorganic compounds. Pd(PPh₃)₂Cl₂, Pd(OAc)₂, PdCl₂ or Na₂PdCl₄ may be mentioned by way of example. Here, Ph is phenyl; Ac is acetyl.

The different catalysts can be employed either individually or else as mixtures. In a preferred embodiment of the invention, Pd(PPh₃)₂Cl₂ is used.

To prepare the compound I in which R¹ is CN, the compound Ia in which L is chlorine, bromine or iodine can also be reacted with copper cyanide, analogously to known processes (see, for example, Organikum, 21. edition, 2001, Wiley, p. 404, Tetrahedron Lett. 42, 2001, p. 7473 or Org. Lett. 5, 2003, 1785 and the literature cited therein).

These conversions are usually carried out at temperatures in the range of from 100° C. to the boiling point of the reaction mixture, preferably at from 100° C. to 250° C. In general, the reaction is carried out in an inert organic solvent. Suitable solvents are in particular aprotic polar solvents, for example dimethylformamide, N-methylpyrrolidone, N,N′-dimethylimidazolidin-2-one and dimethylacetamide.

Alternatively, the conversion of group R¹ can also be carried out on the precursors of the compound I. Thus, for example, compounds II in which R¹ is a halogen atom such as chlorine, bromine or iodine can be subjected to the reaction described above.

Alternatively, the alkylation or acylation of the group NR^{4a}, NR^{5a} in which R^{4a} or R^{5a} is H can also be carried out using the precursors, Thus, for example, compounds II, IV, V, VI, VIII in which R^{5a} or R^{5b} is H can be N-alkylated or N-acylated as described above. In the same manner, it is possible to alkylate the precursors II, IV, V, VI, VII in which the radical referred to as R⁴ or R^{4a} is hydrogen.

The compounds I and their agriculturally useful salts are suitable, both in the form of isomer mixtures and in the form of the pure isomers, as herbicides. They are suitable as such or as an appropriately formulated composition. The herbicidal compositions comprising the compound I or Ia control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

Depending on the application method in question, the compounds I or Ia, or compositions comprising them, can additionally be employed in a further number of crop plants for eliminating undesirable plants. Examples of suitable crops are the following:

Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (S. vulgare), Theobroma cacao, Trifolium pratense, Triticum aestivurn, Triticale, Triticum durum, Vicia faba, Vitis vinifera and Zea mays.

Preferred crops are the following: Arachis hypogaea, Beta vulgaris spec. altissima, Brassica napus var. napus, Brassica oleracea, Citrus limon, Citrus sinensis, Coffea arabica (Coffea canephora, Coffea liberica), Cynodon dactylon, Glycine max, Gossypium hirsutum, (Gossypium arboreum, Gossypium herbaceum, Gossypium vitifolium), Helianthus annuus, Hordeum vulgare, Juglans regia, Lens culinaris, Linum usitatissimum, Lycopersicon lycopersicum, Malus spec., Medicago sativa, Nicotiana tabacum (N. rustica), Olea europaea, Oryza sativa, Phaseolus lunatus, Phaseolus vulgaris, Pistacia vera, Pisum sativum, Prunus dulcis, Saccharum officinarum, Secale cereale, Solanum tuberosum, Sorghum bicolor (S. vulgare), Triticale, Triticum aestivum, Triticum durum, Vicia faba, Vitis vinifera and Zea mays

In addition, the compounds of the formula I may also be used in crops which tolerate the action of herbicides owing to breeding, including genetic engineering methods.

In addition, the compounds of the formula I can also be used in crops which tolerate insects or fungal attack as the result of breeding, including genetic engineering methods.

Furthermore, it has been found that the compounds of the formula I are also suitable for the defoliation and/or desiccation of plant parts, for which crop plants such as cotton, potato, oilseed rape, sunflower, soybean or field beans, in particular cotton, are suitable. In this regard, there have been found compositions for the desiccation and/or defoliation of plants, processes for preparing these compositions and methods for desiccating and/or defoliating plants using the compounds of the formula I.

As desiccants, the compounds of the formula I are particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pernicious fruit, stone fruit and nuts. The same mechanism, i.e. the promotion of the development of abscission tissue between fruit part or leaf part and shoot part of the plants is also essential for the controlled defoliation of useful plants, in particular cotton.

Moreover, a shortening of the time interval in which the individual cotton plants mature leads to an increased fiber quality after harvesting.

The compounds I, or the herbicidal compositions comprising the compounds I, can be used, for example, in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading, watering or treatment of the seed or mixing with the seed. The use forms depend on the intended purpose; in any case, they should ensure the finest possible distribution of the active ingredients according to the invention.

The herbicidal compositions comprise a herbicidally effective amount of at least one compound of the formula I or an agriculturally useful salt of I, and auxiliaries which are customary for the formulation of crop protection agents.

Examples of auxiliaries customary for the formulation of crop protection agents are inert auxiliaries, solid carriers, surfactants (such as dispersants, protective colloids, emulsifiers, wetting agents and tackifiers), organic and inorganic thickeners, bactericides, antifreeze agents, antifoams, optionally colorants and, for seed formulations, adhesives.

Examples of thickeners (i.e. compounds which impart to the formulation modified flow properties, i.e. high viscosity in the state of rest and low viscosity in motion) are polysaccharides, such as xanthan gum (Keizan® from Kelco), Rhodopol® 23 (Rhone Poulenc) or Veegurn® (from R. T. Vanderbilt), and also organic and inorganic sheet minerals, such as Attaclay® (from Engelhardt).

Examples of antifoams are silicone emulsions (such as, for example, Silikon® SRE, Wacker or Rhodorsil® from Rhodia), long-chain alcohols, fatty acids, salts of fatty acids, organofluorine compounds and mixtures thereof.

Bactericides can be added for stabilizing the aqueous herbicidal formulations. Examples of bactericides are bactericides based on dichlorophen and benzyl alcohol hemiformal (Proxel® from ICI or Acticide® RS from Thor Chemie and Kathon® MK from Rohm & Haas), and also isothiazolinone derivates, such as alkylisothiazolinones and benzisothiazolinones (Acticide MBS from Thor Chemie).

Examples of antifreeze agents are ethylene glycol, propylene glycol, urea or glycerol.

Examples of colorants are both sparingly water-soluble pigments and water-soluble dyes. Examples which may be mentioned are the dyes known under the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of adhesives are polyvinylpyrrolidone, polyvinyl acetate, polyvinyl alcohol and tylose.

Suitable inert auxiliaries are, for example, the following:

mineral oil fractions of medium to high boiling point, such as kerosene and diesel oil, furthermore coal tar oils and oils of vegetable or animal origin, aliphatic, cyclic and aromatic hydrocarbons, for example paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone or strongly polar solvents, for example amines such as N-methylpyrrolidone, and water.

Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

Suitable surfactants (adjuvants, wetting agents, tackifiers, dispersants and also emulsifiers) are the alkali metal salts, alkaline earth metal salts and ammonium salts of aromatic sulfonic acids, for example lignosulfonic acids (e.g. Borrespers-types, Borregaard), phenolsulfonic acids, naphthalenesulfonic acids (Morwet types, Akzo Nobel) and dibutylnaphthalenesulfonic acid (Nekal types, BASF AG), and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denaturated proteins, polysaccharides (e.g. methylcellulose), hydrophobically modified starches, polyvinyl alcohol (Mowiol types Clariant), polycarboxylates (BASF AG, Sokalan types), polyalkoxylates, polyvinylamine (BASF AG, Lupamine types), polyethyleneimine (BASF AG, Lupasol types), polyvinylpyrrolidone and copolymers thereof.

Powders, materials for broadcasting and dusts can be prepared by mixing or grinding the active ingredients together with a solid carrier.

Granules, for example coated granules, impregnated granules and homogeneous granules, can be prepared by binding the active ingredients to solid carriers.

Aqueous use forms can be prepared from emulsion concentrates, suspensions, pastes, wettable powders or water-dispersible granules by adding water. To prepare emulsions, pastes or oil dispersions, the compounds of the formula I or Ia, either as such or dissolved in an oil or solvent, can be homogenized in water by means of a wetting agent, tackifier, dispersant or emulsifier. Alternatively, it is also possible to prepare concentrates comprising active compound, wetting agent, tackifier, dispersant or emulsifier and, if desired, solvent or oil, which are suitable for dilution with water. The concentrations of the compounds of the formula I in the ready-to-use preparations can be varied within wide ranges. In general, the formulations comprise approximately from 0.001 to 98% by weight, preferably 0.01 to 95% by weight of at least one active ingredient. The active ingredients are employed in a purity of from 90% to 100%, preferably 95% to 100% (according to NMR spectrum).

The compounds I of the invention can for example be formulated as follows:

1. Products for Dilution with Water

### A Water-Soluble Concentrates

10 pails by weight of active compound are dissolved in 90 parts by weight of water or a water-soluble solvent. As an alternative, wetters or other adjuvants are added. The active compound dissolves upon dilution with water. This gives a formulation with an active compound content of 10% by weight.

### B Dispersible Concentrates

20 parts by weight of active compound are dissolved in 70 parts by weight of cyclohexanone with addition of 10 parts by weight of a dispersant, for example polyvinylpyrrolidone. Dilution with water gives a dispersion. The active compound content is 20% by weight.

### C Emulsifiable Concentrates

15 parts by weight of active compound are dissolved in 75 parts by weight of an organic solvent (eg. alkylaromatics) with addition of calcium dodecylbenzenesulfonate and castor oil ethoxylate (in each case 5 parts by weight). Dilution with water gives an emulsion. The formulation has an active compound content of 15% by weight.

### D Emulsions

25 parts by weight of active compound are dissolved in 35 parts by weight of an organic solvent (eg. alkylaromatics) with addition of calcium dodecylbenzenesulfonate and castor oil ethoxylate (in each case 5 parts by weight). This mixture is introduced into 30 parts by weight of water by means of an emulsifier (Ultraturrax) and made into a homogeneous emulsion. Dilution with water gives an emulsion. The formulation has an active compound content of 25% by weight.

### E Suspensions

In an agitated ball mill, 20 parts by weight of active compound are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules

50 parts by weight of active compound are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders

75 parts by weight of active compound are ground in a rotor-stator mill with addition of 25 parts by weight of dispersants, wetters and silica gel. Dilution with water gives a stable dispersion or solution of the active compound. The active compound content of the formulation is 75% by weight.

### H Gel Formulations

In a ball mill, 20 parts by weight of active compound, 10 parts by weight of dispersant, 1 part by weight of gelling agent and 70 parts by weight of water or of an organic solvent are mixed to give a fine suspension. Dilution with water gives a stable suspension with active compound content of 20% by weight.

### 2. Products to be Applied Undiluted

### I Dusts

5 parts by weight of active compound are ground finely and mixed intimately with 95 parks by weight of finely divided kaolin. This gives a dusting powder with an active compound content of 5% by weight.

### J Granules (GR, FG, GG, MG)

0.5 parts by weight of active compound are ground finely and associated with 99.5 parts by weight of carriers. Current methods here are extrusion, spray-drying or the fluidized bed. This gives granules to be applied undiluted with an active compound content of 0.5% by weight.

### K ULV Solutions (UL)

10 parts by weight of active compound are dissolved in 90 parts by weight of an organic solvent, for example xylene. This gives a product to be applied undiluted with an active compound content of 10% by weight.

The compounds of the formula I or the herbicidal compositions comprising them can be applied pre- or post-emergence, or together with the seed of a crop plant. It is also possible to apply the herbicidal composition or active compounds by applying seed, pretreated with the herbicidal compositions or active compounds, of a crop plant. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

In a further embodiment, the compounds of the formula I or the herbicidal compositions can be applied by treating seed.

The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the compounds of the formula I according to the invention or the compositions prepared therefrom. Here, the herbicidal compositions can be applied diluted or undiluted.

The term seed comprises seed of all types, such as, for example, corns, seeds, fruits, tubers, seedlings and similar forms. Here, preferably, the term seed describes corns and seeds.

The seed used can be seed of the useful plants mentioned above, but also the seed of transgenic plants or plants obtained by customary breeding methods.

The rates of application of the active compound are from 0.001 to 3.0, preferably 0.01 to 1.0, kg/ha of active substance (a.s.), depending on the control target, the season, the target plants and the growth stage. To treat the seed, the compounds I are generally employed in amounts of from 0.001 to 10 kg per 100 kg of seed.

To widen the spectrum of action and to achieve synergistic effects, the compounds of the formula I may be mixed with a large number of representatives of other herbicidal or growth-regulating active ingredient groups or with safeners and then applied concomitantly. Suitable representatives of other herbicidal or growth-regulating active ingredient groups for mixtures are, for example, 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-aroyl-1,3-cyclohexanediones, 2-hetaroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils, phenyl pyrazolines and isoxazolines and derivatives thereof.

Moreover, it may be useful to apply the compounds of the formula I in combination with safeners. Safeners are chemical compounds which prevent or reduce damage on useful plants without having a major impact on the herbicidal action of the compounds of the formula I towards unwanted plants. They can be applied either before sowings (e.g. on seed treatments, shoots or seedlings) or in the pre-emergence application or post-emergence application of the useful plant. The safeners and the compounds of the formula I can be applied simultaneously or in succession. Suitable safener are e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]-sulfonyl]-2-benzoic amides, 1,8-naphthalic anhydride, 2-halo-4-(haloalkyl)-5-thiazol carboxylic acids, phosphorthiolates and N-alkyl-O-phenylcarbamates and their agriculturally acceptable salts and their agriculturally acceptable derivatives such amides, esters, and thioesters, provided they have an acid group.

It may furthermore be beneficial to apply the compounds of the formula I alone or in combination with other herbicides, or else in the form of a mixture with other crop protection agents, for example together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates may also be added.

Hereinbelow, the preparation of piperazine compounds of the formula I is illustrated by examples; however, the subject matter of the present invention is not limited to the examples given.

### EXAMPLES

The products shown below were characterized by determination of the melting point, by

NMR spectroscopy or by the masses determined by HPLC-MS spectrometry ([m/z]) or by the retention time (RT; [min.]).

[HPLC-MS=high performance liquid chromatography coupled with mass spectrometry; HPLC column: RP-18 column (Chromolith Speed ROD from Merck KgaA, Germany), 50×4.6 mm; mobile phase: acetonitrile+0.1% trifluoroacetic acid (TFA)/water+0.1% TFA, gradient from 5:95 to 100:0 over 5 minutes at 40° C., flow rate 1.8 ml/min;

MS: quadrupole electrospray ionisation, 80 V (positive mode)].

### I. Preparation Examples

### Example 1

### 3-Benzyl-6-(2-bromobenzylidene)-1,3,4-trimethylpiperazine-2,5-dione

### 1.1 Preparation of methyl (2-tert-butoxycarbonylamino-3-phenylpropionylamino)-acetate

At 0° C., ethyldiisopropylamine (259 g, 2.0 mol), N-tert-butoxycarbonyl-L-phenyl-alanine (212 g, 0.8 mol) and 1-ethyl-3-(3′-dimethylaminopropyl)carbodiimide (EDAC, 230 g, 1.2 mol) were added to a solution of glycine methyl ester hydrochloride (100 g, 0.8 mol) in tetrahydrofuran (THF, 1000 ml). The reaction mixture was then stirred at room temperature for 24 h. The reaction mixture obtained was freed under reduced pressure from volatile components, and the residue obtained in this manner was taken up in water (1000 ml). The aqueous phase was extracted repeatedly with CH₂Cl₂. The organic phases obtained in this manner were combined, washed with water, dried over Na₂SO₄, filtered and freed from the solvent under reduced pressure. Methyl (2-tert-butoxycarbonylamino-3-phenylpropionylamino)acetate was obtained as a yellow oil in an amount of 300 g. The crude product obtained was reacted further without further purification.

### 1.2 Preparation of 3-benzylpiperazine-2,5-dione

At room temperature, trifluoroacetic acid (342 g, 3 mol) was added dropwise to a solution of methyl (2-tert-butoxycarbonylamino-3-phenylpropionylamino)acetate (300 q, about 0.8 mol) in CH₂Cl₂. The reaction mixture obtained was stirred at room temperature for 24 h and then concentrated under reduced pressure. The residue obtained was taken up in THF (500 ml), and an aqueous solution of ammonia (25% strength, 500 ml) was added slowly. The reaction mixture was stirred at room temperature for a further 72 h. The precipitated solid was isolated by filtration and washed with water. 3-Benzylpiperazine-2,5-dione was obtained in an amount of 88 g (yield 54%).

### 1.3 Preparation of 1,4-diacetyl-3-benzyl-piperazine-2,5-dione

A solution of 3-benzylpiperazine-2,5-dione (20.4 g, 0.1 mol) in acetic anhydride (200 ml) was stirred under reflux conditions for 4 h. The reaction mixture obtained was concentrated under reduced pressure. The residue was taken up in CH₂Cl₂, washed successively with an aqueous NaHCO₃ solution and water, dried over Na₂SO₄, filtered and freed from the solvent under reduced pressure. 1,4-Diacetyl-3-benzylpiperazine-2,5-dione was obtained as a yellow oil in an amount of 28.5 g (quantitative) and reacted further as crude product.

HPLC-MS [m/z]: 289.1 [M+1]⁺.

### 1.4 Preparation of 1-acetyl-6-benzyl-3-(2-bromobenzylidene)piperazine-2,5-dione

Bromobenzaldehyde (5.55 g, 0.03 mol) and Cs₂CO₃ (9.8 g, 0.03 mol) were added to a solution of 1,4-diacetyl-3-benzylpiperazine-2,5-dione (17.4 g, 0.06 mol) in dimethylformamide (DMF, 100 ml). The reaction mixture was stirred at room temperature for 36 h, water (500 ml) and citric acid (10 g) were then added and the mixture was extracted repeatedly with CH₂Cl₂. The organic phases obtained in this manner were combined, washed with water, dried over Na₂SO₄, filtered and freed from the solvent under reduced pressure. After purification by column chromatography (mobile phase: CH₂Cl₂), 1-acetyl-6-benzyl-3-(2-bromobenzylidene)piperazine-2,5-dione was obtained as a yellow oil in an amount of 12 g (yield 48%).

HPLC-MS [m/z]: 413.9 [M+1]⁺.

### 1.5 Preparation of 3-benzyl-6-(2-bromobenzylidene)-piperazine-2,5-dione

Dilute aqueous hydrochloric acid (5% strength, 250 ml) was added to a solution of 1-acetyl-6-benzyl-3-(2-bromobenzylidene)piperazine-2,5-dione (12 g, 0.03 mol) in THF (50 ml). The reaction mixture was stirred under reflux conditions for 8 h. After cooling of the reaction solution, the precipitated solid was isolated by filtration. The solid obtained in this manner was washed with water and THF. 3-Benzyl-6-(2-bromobenzylidene)piperazine-2,5-dione was obtained as a colorless solid in an amount of 8.3 g (yield 75%).

HPLC-MS [m/z]: 371.2 [M]⁺.

### 1.6 3-Benzyl-6-(2-bromobenzylidene)-1,3,4-trimethylpiperazine-2,5-dione

At 0° C., NaH (0.85 g, 60% pure, 21 mmol) was added to a solution of 3-benzyl-6-(2-bromobenzylidene)piperazine-2,5-dione (2.00 g, 5.4 mmol) in DMF (50 ml). The reaction mixture was stirred at 0° C. for 2 h, and methyl iodide (5.0 g, 35 mmol) was then added. The reaction mixture was stirred at room temperature for a further 18 h, and water was then added. The mixture was extracted repeatedly with methyl tert-butyl ether. The organic phases obtained in this manner were combined, washed with water, dried over Na₂SO₄, filtered and freed from the solvent under reduced pressure. After purification by column chromatography, 3-benzyl-6-(2-bromobenzylidene)-1,3,4-trimethylpiperazine-2,5-dione was obtained in an amount of 1.6 g (yield 72%).

HPLC-MS [m/z]: 413.0 [M]⁺.

### Example 2

### 2-(5-Benzyl-1,4,5-trimethyl-3,6-dioxopiperazin-2-ylidenemethyl)benzonitrile

CuCN (0.7 g, 7.8 mmol) was added to a solution of 3-benzyl-6-(2-bromo-benzylidene)-1,3,4-trimethylpiperazine-2,5-dione (1.5 g, 3.6 mmol) in N-methylpyrrolidin (NMP, 25 ml). The reaction mixture was stirred at 155 C for 16 h and, after cooling to room temperature, introduced into ethyl acetate. The reaction mixture was diluted with methyl tert-butyl ether. The organic phase obtained in this manner was washed with water, dried over Na₂SO₄, filtered and freed from the solvent under reduced pressure. Purification by column chromatography gave 2-(5-benzyl-1,4,5-trimethyl-3,6-dioxopiperazin-2-ylidenemethyl)benzonitrile in an amount of 0.79 g (yield 61%).

HPLC-MS [m/z]: 360.5 [M+1]⁺.

### Example 3

### 2-(5-Benzyl-5-ethyl-1,4-dimethyl-3,6-dioxopiperazin-2-ylidenemethyl)-benzonitrile

### 3.1 Preparation of 3-benzyl-6-(2-bromobenzyliden)-1,4-dimethylpiperazine-2,5-dione

At 0° C., NaH (0.8 g, 60% pure, 0.02 mol) was added to a solution of 3-benzyl-6-(2-bromobenzylidene)piperazine-2,5-dione (3.71 g, 0.01 mol) in DMF (50 ml). The mixture was stirred at 0° C. for 1 h, and methyl iodide (14.2 g, 0.1 mol) was then added. The reaction mixture obtained was stirred at room temperature for a further 18 h and then introduced into a water (500 ml)/citric acid (5 g) solution, The reaction mixture obtained was extracted repeatedly with CH₂Cl₂. The organic phases obtained in this manner were combined, washed with water, dried over Na₂SO₄, filtered and freed from the solvent under reduced pressure. After trituration with diisopropyl ether, 3-benzyl-6-(2-bromobenzylidene)-1,4-dimethylpiperazine-2,5-dione was obtained in an amount of 2 g (yield 50%).

HPLC-MS [m/z]: 401.4 [M+1]⁺.

### 3.2 Preparation of 2-(5-benzyl-1,4-dimethyl-3,6-dioxopiperazin-2-ylidenemethyl)-benzonitrile

CuCN (0.9 g, 0.1 mol) was added to a solution of 3-benzyl-6-(2-bromobenzylidene)-1,4-dimethylpiperazine-2,5-dione (2 g, 0.005 mol) in NMP (20 ml). The reaction mixture was stirred at 150° C. for 18 h and then introduced into an aqueous NaCN solution (6% strength, 50 ml). The reaction mixture was extracted repeatedly with CH₂Cl₂. The organic phases obtained in this manner were combined, washed with water, dried over Na₂SO₄, filtered and freed from the solvent under reduced pressure. After purification by column chromatography and trituration with diisopropyl ether, 2-(5-benzyl-1,4-dimethyl-3,6-dioxopiperazin-2-ylidenemethyl)benzonitrile was obtained as a beige solid in an amount of 1.2 g (yield 67%).

HPLC-MS [m/z]: 346.4 [M+1]⁺.

### 3.3 Preparation of 2-(5-benzyl-5-ethyl-1,4-dimethyl-3,6-dioxopiperazin-2-ylidenemethyl)benzonitrile

At 0° C., NaH (0.12 g, 60% pure, about 3 mmol) was added to a solution of 2-(5-benzyl-1,4-dimethyl-3,6-dioxopiperazin-2-ylidenemethyl)benzonitrile (1.04 g, 3.0 mmol) in DMF (10 ml). The reaction mixture was stirred at 0° C. for 1 h, and iodoethane (0.47 g, 3.1 mmol) was then added. The reaction mixture obtained was stirred at room temperature for 18 h and then introduced into water (100 ml) and acidified. The mixture was extracted 3 times with dichloromethane, and the combined organic phases were washed with water and dried over sodium sulfate. Concentration of the dried organic phase gave 1.2 g of the title compound as a crude product. The crude product was treated initially with n-hexane and then with hot ethyl acetate. The solid residue was purified by flash chromatography using ethyl acetate as mobile phase. In this manner, 200 mg of the title compound were obtained as a white solid (200 mg, Z isomer, melting point 141° C.). Work-up of the mother liquor obtained on trituration with ethyl acetate gave a further 400 mg of the title compound as an E/Z isomer mixture having a melting point of 120° C. (E/Z about 1:1).

The preparation of the compounds of the formula I compiled in Tables 1, 2 and 3 (Examples 4 to 190) was carried out analogously to Examples 1 to 3 shown above.

TABLE 1
Compounds of the general formula I in which R⁴ is CH₃ and R⁷, R⁸, R⁹ and R¹⁰
are each hydrogen (compounds of the formula I.b)

| | |
|---|---|
| | (I.b) |
| 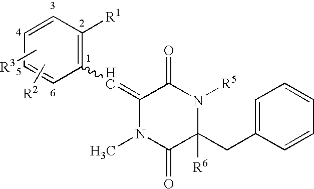 | |

| | | | | | | RT, [m/z] | |
|---|---|---|---|---|---|---|---|
| Ex. | R¹ | R² | R³ | R⁵ | R⁶ | and/or m.p. | Isomer*) |
| 4 | CN | 5-CN | 6-Cl | CH₃ | CH₃ | 3.246 min | Z |
| | | | | | | m/z = 419.5 | |
| | | | | | | [M + H]⁺ | |
| 5 | Br | 5-Cl | 6-Cl | CH₃ | CH₃ | 3.933 min | Z |
| | | | | | | m/z = 482.8 | |
| | | | | | | [M]⁺ | |
| 6 | CN | 5-Cl | 6-Cl | CH₃ | CH₃ | 3.576 min | Z |
| | | | | | | m/z = 428.4 | |
| | | | | | | [M]⁺ | |
| 7 | CN | H | H | CH₃ | CH₃ | 3.014 min | Z |
| | | | | | | m/z = 360.5 | |
| | | | | | | [M + H]⁺ | |
| | | | | | | 160-162° C. | |
| 8**⁾ | CN | H | H | CH₃ | CH₃ | 136° C. | Z |
| 9 | CN | H | H | CH₃ | CH₃ | 2.871 min | E |
| | | | | | | m/z = 360.0 | |
| | | | | | | [M + H]⁺ | |
| 10 | Br | H | H | CH₃ | CH₃ | 3.425 min | Z |
| | | | | | | m/z = 413.0 | |
| | | | | | | [M]⁺ | |
| 11 | Br | 6-Cl | H | CH₃ | CH₃ | 3.563 min | Z |
| | | | | | | m/z = 448.8 | |
| | | | | | | [M + H]⁺ | |
| 12 | CN | 6-F | H | CH₃ | CH₃ | 3.092 min | Z |
| | | | | | | m/z = 378.3 | |
| | | | | | | [M + H]⁺ | |
| | | | | | | 88-90° C. | |
| 13 | CN | 6-Cl | H | CH₃ | CH₃ | 3.246 min | Z |
| | | | | | | m/z = 394.4 | |
| | | | | | | [M + H]⁺ | |
| 14 | CN | 6-OCH₃ | H | CH₃ | CH₃ | 3.121 min | Z |
| | | | | | | m/z = 390.4 | |
| | | | | | | [M]⁺ | |
| | | | | | | 116° C. | |
| 15 | CN | 6-OCH₃ | H | CH₃ | CH₃ | 3.006 min | Z |
| | | | | | | m/z = 390.4 | |
| | | | | | | [M + H]⁺ | |
| 16 | CN | 6-CH₃ | H | CH₃ | CH₃ | 3.110 min | Z |
| | | | | | | m/z = 374.4 | |
| | | | | | | [M + H]⁺ | |
| 17 | CN | 6-CH₃ | H | CH₃ | CH₃ | 3.204 min | E |
| | | | | | | m/z = 374.4 | |
| | | | | | | [M + H]⁺ | |
| 18 | CN | 6-F | 5-F | CH₃ | CH₃ | 3.170 min | Z |
| | | | | | | m/z = 396.0 | |
| | | | | | | [M + H]⁺ | |
| | | | | | | 58° C. | |
| 19 | CN | 6-n-butyl | H | CH₃ | CH₃ | 3.739 | Z |
| | | | | | | m/z = 416.5 | |
| | | | | | | [M + H]⁺ | |
| | | | | | | 58° C. | |
| 20 | Br | 6-F | 5-F | CH₃ | CH₃ | 3.494 min | Z |
| | | | | | | m/z = 450.8 | |
| | | | | | | [M + H]⁺ | |
| 21 | Br | 6-allyl | H | CH₃ | CH₃ | 3.767 min | Z |
| | | | | | | m/z = 455.4 | |
| | | | | | | [M + H]⁺ | |
| 22 | CN | H | H | CH₃ | n-propyl | 3.320 min | Z |
| | | | | | | m/z = 388.4 | |
| | | | | | | [M + H]⁺ | |
| | | | | | | 142° C. | |
| 23 | CN | 6-allyl | H | CH₃ | CH₃ | 3.433 min | Z |
| | | | | | | m/z = 400.4 | |
| | | | | | | [M + H]⁺ | |
| | | | | | | 125° C. | |
| 24 | CN | H | H | CH₃ | isopropyl | 3.319 min | Z |
| | | | | | | m/z = 388.0 | |
| | | | | | | [M + H]⁺ | |
| | | | | | | 55° C. | |
| 25 | CN | H | H | CH₃ | —CH₂OH | 2.625 min | Z |
| | | | | | | m/z = 376.4 | |
| | | | | | | [M + H]⁺ | |
| | | | | | | 134° C. | |
| 26 | NO₂ | H | H | CH₃ | CH₃ | 2.980 min | Z:E = |
| | | | | | | m/z = 379.9 | 60:40 |
| | | | | | | [M + H]⁺ | |
| 27 | NO₂ | H | H | CH₃ | CH₃ | 152° C. | Z- |
| 28 | NO₂ | H | H | CH₃ | CH₃ | 106° C. | Z:E = |
| | | | | | | | 9:1 |
| 28**⁾ | CN | 6-n-propyl | H | CH₃ | CH₃ | 3.665 min | Z |
| | | | | | | m/z = 402.0 | |
| | | | | | | [M + H]⁺ | |
| 29 | CN | 6-ethyl | H | CH₃ | CH₃ | 3.315 min | Z |
| | | | | | | m/z = 388.0 | |
| | | | | | | [M + H]⁺ | |
| | | | | | | 74-76° C. | |
| 30 | CN | 6-benzyl | H | CH₃ | CH₃ | 3.613 min | Z |
| | | | | | | m/z = 450.0 | |
| | | | | | | [M + H]⁺ | |
| | | | | | | 152-153° C. | |
| 31 | Br | 6-F | H | CH₃ | CH₃ | 130-132° C. | Z |
| 32 | Br | H | H | CH₃ | CH₃ | 3.359 min | Z |
| | | | | | | m/z = 431.4 | |
| | | | | | | [M]⁺ | |
| 33 | Cl | 6-SCH₃ | H | CH₃ | CH₃ | 3.484 min | Z |
| | | | | | | m/z = 414.9 | |
| | | | | | | [M]⁺ | |
| | | | | | | 60-62° C. | |
| 34 | Br | 6-benzyl | H | CH₃ | CH₃ | 3.944 min | Z |
| | | | | | | m/z = 504.9 | |
| | | | | | | [M + H]⁺ | |
| 35 | Br | 6-n-butyl | H | CH₃ | CH₃ | 4.095 min | Z |
| | | | | | | m/z = 470.9 | |
| | | | | | | [M + H]⁺ | |
| 36 | Br | 6-n-propyl | H | CH₃ | CH₃ | 3.906 min | Z |
| | | | | | | m/z = 455.4 | |
| | | | | | | [M]⁺ | |
| 37 | CN | 6-SCH₃ | H | CH₃ | CH₃ | 3.429 min | Z |
| | | | | | | m/z = 406.1 | |
| | | | | | | [M + H]⁺ | |
| | | | | | | 168° C. | |
| 38 | Cl | H | H | CH₃ | CH₃ | 3.503 min | Z |
| | | | | | | m/z = 369.1 | |
| | | | | | | [M + H]⁺ | |
| | | | | | | 151° C. | |
| 39 | CN | H | H | H | CH₃ | 2.752 min | Z |
| | | | | | | m/z = 346.4 | |
| | | | | | | [M + H]⁺ | |
| | | | | | | 133° C. | |
| 40 | CN | H | H | H | CH₃ | 65° C. | Z |
| 41**) | Cl | 6-SO₂CH₃ | H | CH₃ | CH₃ | 3.163 min | Z |
| | | | | | | m/z = 447.0 | |
| | | | | | | [M]⁺ | |
| 42 | CN | 6-SO₂CH₃ | H | CH₃ | CH₃ | 2.897 min | Z |
| | | | | | | m/z = 438.1 | |
| | | | | | | [M + H]⁺ | |
| 43 | Br | 6-OCH₃ | H | CH₃ | CH₃ | 3.537 min | Z |
| | | | | | | m/z = 445.0 | |
| | | | | | | [M]⁺ | |
| | | | | | | 105° C. | |
| 44 | NO₂ | H | H | H | CH₃ | 2.900 min | Z |
| | | | | | | m/z = 366.1 | |
| | | | | | | [M + H]⁺ | |
| | | | | | | 150° C. | |
| 45 | NO₂ | H | H | C(O)CH₃ | CH₃ | 3.678 min | Z |
| | | | | | | m/z = 430.0 | |
| | | | | | | [M + Na]⁺ | |
| | | | | | | 157° C. | |
| 46 | CN | 6-S(O)CH₃ | H | CH₃ | CH₃ | 2.618 min | Z |
| | | | | | | m/z = 422.1 | |
| | | | | | | [M + H]⁺ | |
| 47 | NO₂ | H | H | CH₂CH₃ | CH₃ | 3.290 min | Z |
| | | | | | | m/z = 394.1 | |
| | | | | | | [M + H]⁺ | |
| | | | | | | 123° C. | |
| 48 | NO₂ | 4-CF₃ | H | CH₃ | CH₃ | 3.670 min | Z |
| | | | | | | m/z = 447.9 | |
| | | | | | | [M + H]⁺ | |
| 49 | NO₂ | 3-OCH₃ | H | CH₃ | CH₃ | 3.327 min | Z |
| | | | | | | m/z = 409.9 | |
| | | | | | | [M + H]⁺ | |
| 50 | NO₂ | 4-Cl | H | CH₃ | CH₃ | 3.563 min | Z |
| | | | | | | m/z = 413.9 | |
| | | | | | | [M]⁺ | |
| 51 | NO₂ | 3-OCH₃ | H | CH₃ | CH₃ | 3.318 min | Z |
| | | | | | | m/z = 409.9 | |
| | | | | | | [M + H]⁺ | |
| 52 | J | H | H | CH₃ | CH₃ | 3.455 min | Z |
| | | | | | | m/z = 461.4 | |
| | | | | | | [M + H]⁺ | |
| | | | | | | 169-171° C. | |
| 53 | J | H | H | CH₃ | CH₃ | 3.392 min | E |
| | | | | | | m/z = 460.8 | |
| | | | | | | [M + H]⁺ | |
| | | | | | | 186-187° C. | |
| 54 | CHO | H | H | CH₃ | CH₃ | 2.879 min | Z |
| | | | | | | m/z = 363.4 | |
| | | | | | | [M + H]⁺ | |
| 55 | 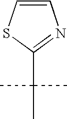 | H | H | CH₃ | CH₃ | 3.178 min m/z = 418.4 [M + H]⁺ 82-90° C. | Z |
| 56 | NO₂ | 6-CH₃ | H | CH₃ | CH₃ | 3.411 min | Z |
| | | | | | | m/z = 394.1 | |
| | | | | | | [M + H]⁺ | |
| 57 | 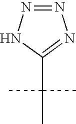 | H | H | CH₃ | CH₃ | 2.698 min m/z = 403.1 [M + H]⁺ 198-200° C. | Z |
| 58 | 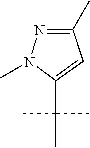 | H | H | CH₃ | CH₃ | 3.042 min m/z = 429.1 [M + H]⁺ | Z |
| 59 | 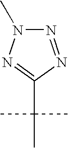 | H | H | CH₃ | CH₃ | 3.049 min m/z = 417.1 [M + H]⁺ | Z |
| 60 | 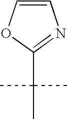 | H | H | CH₃ | CH₃ | 3.092 min m/z = 402.1 [M + H]⁺ | -Z |
| 61 | 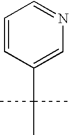 | H | H | CH₃ | CH₃ | 2.452 min m/z = 412.1 [M + H]⁺ | Z |
| 62 | 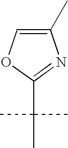 | H | H | CH₃ | CH₃ | 3.315 min m/z = 416.1 [M + H]⁺ | Z |
| 63 | 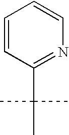 | H | H | CH₃ | CH₃ | 2.368 min m/z = 412.1 [M + H]⁺ | Z |
| 64 | 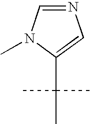 | H | H | CH₃ | CH₃ | 2.352 min m/z = 415.0 [M + H]⁺ 193-194° C. | Z |
| 65 | 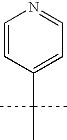 | H | H | CH₃ | CH₃ | 2.394 min m/z = 412.1 [M + H]⁺ | Z |
| 66 | NO₂ | 5-F | H | CH₃ | CH₃ | 3.192 min | Z |
| | | | | | | m/z = 398.1 | |
| | | | | | | [M + H]⁺ | |
| 67 | 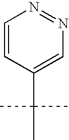 | H | H | CH₃ | CH₃ | 2.655 min m/z = 413.1 [M + H]⁺ | Z |
| 68 | 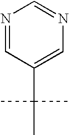 | H | H | CH₃ | CH₃ | 2.873 min m/z = 413.1 [M + H]⁺ | Z |
| 69 | 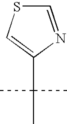 | H | H | CH₃ | CH₃ | 3.208 min m/z = 418.1 [M + H]⁺ | Z |
| 70 | 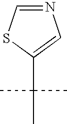 | H | H | CH₃ | CH₃ | 3.130 min m/z = 418.1 [M + H]⁺ | Z |
| 71 | 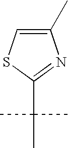 | H | H | CH₃ | CH₃ | 3.304 min m/z = 432.1 [M + H]⁺ | Z |
| 72 | 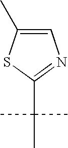 | H | H | CH₃ | CH₃ | 3.420 min m/z = 432.1 [M + H]⁺ | Z |
| 73 | NO₂ | 6-F | H | CH₃ | CH₃ | 3.229 min | Z |
| | | | | | | m/z = 398.1 | |
| | | | | | | [M + H]⁺ | |
| | | | | | | 161° C. | |
| 74 | 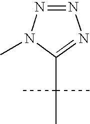 | H | H | CH₃ | CH₃ | 2.794 min m/z = 417.1 [M + H]⁺ | Z |
| 75 | 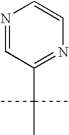 | H | H | CH₃ | CH₃ | 2.954 min m/z = 413.1 [M + H]⁺ | Z |
| 76 | NO₂ | 5-Cl | H | CH₃ | CH₃ | 3.414 min | Z |
| | | | | | | m/z = 414.0 | |
| | | | | | | [M + H]⁺ | |
| 77 | NO₂ | 6-Cl | H | CH₃ | CH₃ | 3.327 min | Z |
| | | | | | | m/z = 414.0 | |
| | | | | | | [M + H]⁺ | |
| 78 | NO₂ | 6-Br | H | CH₃ | CH₃ | 3.356 min | Z |
| | | | | | | m/z = 458.0 | |
| | | | | | | [M + H]⁺ | |
| 79 | CN | 6-Br | H | CH₃ | CH₃ | 3.388 min | Z |
| | | | | | | m/z = 440 | |
| | | | | | | [M + H]⁺ | |
| 80 | CN | 6-CN | H | CH₃ | CH₃ | 3.089 min | Z |
| | | | | | | m/z = 385 | |
| | | | | | | [M + H]⁺ | |
| | | | | | | 133-135° C. | |
| 81 | NO₂ | 5-F | 6-F | CH₃ | CH₃ | 3.266 min | Z |
| | | | | | | m/z = 416 | |
| | | | | | | [M +H]⁺ | |
| | | | | | | 136-138° C. | |
| 82 | NO₂ | 5-OCH₃ | H | CH₃ | CH₃ | 3.115 min | Z |
| | | | | | | m/z = 410 | |
| | | | | | | [M + H]⁺ | |
| | | | | | | 75-80° C. | |
| 83 | NO₂ | 6-CN | H | CH₃ | CH₃ | 3.110 min | Z |
| | | | | | | m/z = 405 | |
| | | | | | | [M + H]⁺ | |
| | | | | | | 157-159° C. | |
| 84 | NO₂ | 2-HC═CH₂ | H | CH₃ | CH₃ | 3.370 min | Z |
| | | | | | | m/z = 406 | |
| | | | | | | [M + H]⁺ | |
| 85 | Br | 5-CF₃ | H | CH₃ | CH₃ | 3.868 min | Z |
| | | | | | | m/z = 483 | |
| | | | | | | [M + H]⁺ | |
| | | | | | | 124-125° C. | |
| 86 | Br | 4-F | H | CH₃ | CH₃ | 3.580 min | Z |
| | | | | | | m/z = 433 | |
| | | | | | | [M + H]⁺ | |
| | | | | | | 134-135° C. | |
| 87 | NO₂ | 3-Cl | H | CH₃ | CH₃ | 3.452 min | Z |
| | | | | | | m/z = 414 | |
| | | | | | | [M + H]⁺ | |
| | | | | | | 95-99° C. | |
| 88 | NO₂ | 6-CF₃ | H | CH₃ | CH₃ | 3.490 min | Z |
| | | | | | | m/z = 448 | |
| | | | | | | [M + H]⁺ | |
| 89 | CN | H | H | H | CH₃ | 2.818 min | Z |
| | | | | | | m/z = 346 | |
| | | | | | | [M + H]⁺ | |
| | | | | | | 132° C. | |
| 90 | NO₂ | 6-OCH₃ | H | CH₃ | CH₃ | 3.328 min | Z |
| | | | | | | m/z = 410 | |
| | | | | | | [M + H]⁺ | |
| | | | | | | 37-40° C. | |
| 91 | 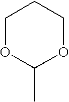 | H | H | CH₃ | CH₃ | 2.872 min m/z = 407 [M + H]⁺ | Z |
| 92 | C₆H₅ | H | H | CH₃ | CH₃ | 3.678 min | Z |
| | | | | | | m/z = 434 | |
| | | | | | | [M + Na]⁺ | |
| 93 | CN | 5-F | H | CH₃ | CH₃ | 3.207 min | Z |
| | | | | | | m/z = 377.4 | |
| | | | | | | [M + H]⁺ | |
| 94 | Br | 3-CH₃ | H | CH₃ | CH₃ | 124-129° C. | Z |
| 95 | Br | 3-F | H | CH₃ | CH₃ | 3.500 min | Z |
| | | | | | | m/z = 431.3 | |
| | | | | | | [M + H]⁺ | |
| | | | | | | 132-135° C. | |
| 96 | CN | 4-CH₃ | H | CH₃ | CH₃ | 3.345 min | Z |
| | | | | | | m/z = 373.4 | |
| | | | | | | [M + H]⁺ | |
| 97 | CN | 3-F | H | CH₃ | CH₃ | 3.216 min | Z |
| | | | | | | m/z = 377.4 | |
| | | | | | | [M + H]⁺ | |
| | | | | | | 153-159° C. | |
| 98 | 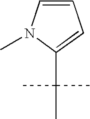 | H | H | CH₃ | CH₃ | 3.597 min m/z = 413.5 [M + H]⁺ 147-152° C. | Z |
| 99 | 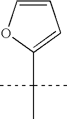 | H | H | CH₃ | CH₃ | 3.555 min m/z = 400.5 [M + H]⁺ 104-110° C. | Z |
| 100 | CN | H | H | CH₃ | CH₂F | 3.112 min | Z |
| | | | | | | m/z = 378.9 | |
| | | | | | | [M + H]⁺ | |
| 101 | NO₂ | 4-CH₃ | H | CH₃ | CH₃ | 3.403 min | Z |
| | | | | | | m/z = 393.5 | |
| | | | | | | [M + H]⁺ | |
| | | | | | | 185° C. | |
| 102 | Br | 5-OCH₃ | H | CH₃ | CH₃ | 3.401 min | Z |
| | | | | | | m/z = 393.5 | |
| | | | | | | [M + H]⁺ | |
| 103 | Br | 5-F | H | CH₃ | CH₃ | 3.622 min | Z |
| | | | | | | m/z = 331.3 | |
| | | | | | | [M + H]⁺ | |
| 104 | CN | 5-OCH₃ | H | CH₃ | CH₃ | 3.033 min | Z |
| | | | | | | m/z = 389.5 | |
| | | | | | | [M + H]⁺ | |
| 105 | 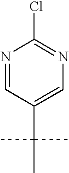 | H | H | CH₃ | CH₃ | 3.190 min m/z = 446.9 [M + H]⁺ | Z |
| 106 | 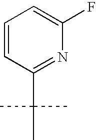 | H | H | CH₃ | CH₃ | 3.338 min m/z = 429.5 [M + H]⁺ 65-70° C. | Z |
| 107 | CN | 5-CF₃ | H | CH₃ | CH₃ | 3.555 min | Z |
| | | | | | | m/z = 427.4 | |
| | | | | | | [M + H]⁺ | |
| 108 | CN | 5-F | H | CH₃ | CH₃ | 3.051 min | Z |
| | | | | | | m/z = 377.4 | |
| | | | | | | [M + H]⁺ | |
| 109 | 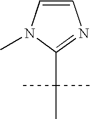 | H | H | CH₃ | CH₃ | 2.411 min m/z = 414.5 [M + H]⁺ | Z |
| 110 | NO₂ | H | H | CH₃ | CH₂F | 3.859 min | Z |
| | | | | | | m/z = 398.1 | |
| | | | | | | [M + H]⁺ | |
| 111 | Br | 5-OCHF₂ | H | CH₃ | CH₃ | 3.576 min | Z |
| | | | | | | m/z = 481.8 | |
| | | | | | | [M + H]⁺ | |
| | | | | | | 125-127° C. | |
| 112 | NO₂ | 5-Br | H | CH₃ | CH₃ | 3.579 min | Z |
| | | | | | | m/z = 458.3 | |
| | | | | | | [M + H]⁺ | |
| | | | | | | 156-160° C. | |
| 113 | CN | 5-OCHF₂ | H | CH₃ | CH₃ | 3.150 min | Z |
| | | | | | | m/z = 426.1 | |
| | | | | | | [M + H]⁺ | |
| | | | | | | 105-107° C. | |
| 114 | CN | 5-SO₂CH₃ | H | CH₃ | CH₃ | 2.798 min | Z |
| | | | | | | m/z = 437.5 | |
| | | | | | | [M + H]⁺ | |
| | | | | | | 100° C. | |
| 115 | CN | 5-SOCH₃ | H | CH₃ | CH₃ | 2.481 min | E |
| | | | | | | m/z = 422.1 | |
| | | | | | | [M + H]⁺ | |
| | | | | | | 92° C. | |
| 116 | CN | 5-SOCH₃ | H | CH₃ | CH₃ | 2.477 min | Z |
| | | | | | | m/z = 422.1 | |
| | | | | | | [M + H]⁺ | |
| 117 | CN | 5-SCH₃ | H | CH₃ | CH₃ | 3.340 min | Z |
| | | | | | | m/z = 406.1 | |
| | | | | | | [M + H]⁺ | |
| | | | | | | 147° C. | |
| 118 | Cl | 5-OCH₃ | 6-F | CH₃ | CH₃ | 3.532 min | Z |
| | | | | | | m/z = 417.1 | |
| | | | | | | [M + H]⁺ | |
| | | | | | | 128-130° C. | |
| 119 | Br | 5-OCH₃ | 6-F | CH₃ | CH₃ | 3.589 min | Z |
| | | | | | | m/z = 463.0 | |
| | | | | | | [M + H]⁺ | |
| | | | | | | 130-132° C. | |
| 120 | CN | 5-OCH₃ | 6-F | CH₃ | CH₃ | 3.155 min | Z |
| | | | | | | m/z = 407.8 | |
| | | | | | | [M + H]⁺ | |
| | | | | | | 131-133° C. | |
| 121 | Cl | 3-CF₃ | H | H | CH₃ | 3.567 min | Z |
| | | | | | | m/z = 423.0 | |
| | | | | | | [M + H]⁺ | |
| 122 | Cl | 3-CF₃ | H | CH₃ | CH₃ | 3.669 min | Z |
| | | | | | | m/z = 436.7 | |
| | | | | | | [M]⁺ | |
| | | | | | | 131° C. | |
| 123 | CN | 3-F | 5-F | CH₃ | CH₃ | 3.247 min | Z |
| | | | | | | m/z = 495.8 | |
| | | | | | | [M]⁺ | |
| 124 | 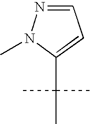 | H | H | CH₃ | CH₃ | 3.039 min m/z = 415.2 [M + H]⁺ | Z |
| 125 | 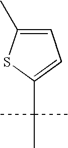 | H | H | CH₃ | CH₃ | 4.065 min m/z = 431.1 [M + H]⁺ | Z |
| 126 | 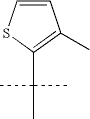 | H | H | CH₃ | CH₃ | 3.868 min m/z = 430.8 [M]⁺ | Z |
| 127 | CN | H | H | CH₂CH₃ | CH₃ | 3.358 min | Z |
| | | | | | | m/z = 374.1 | |
| | | | | | | [M + H]⁺ | |
| 128 | CN | H | H | CH(CH₃)₂ | CH₃ | 4.215 min | Z |
| | | | | | | m/z = 388.1 | |
| | | | | | | [M + H]⁺ | |
| 129 | CN | H | H | butyl | CH₃ | 3.816 min | Z |
| | | | | | | m/z = 402.2 | |
| | | | | | | [M + H]⁺ | |
| 130 | CN | H | H | allyl | CH₃ | 3.472 min | Z |
| | | | | | | m/z = 368.15 | |
| | | | | | | [M + H]⁺ | |
| 131 | Br | H | H | CH₃ | CF₃ | 7.063 min | Z |
| | | | | | | m/z = 467.1 | |
| | | | | | | [M + H]^{+ (1)} | |
| 132 | CN | H | H | CH₃ | CF₃ | 6.257 min | Z |
| | | | | | | m/z = 414.02 | |
| | | | | | | [M + H]^{+ (1)} | |
| 133 | CN | H | H | CH₃ | CF₃ | 6.649 min | E |
| | | | | | | m/z = 414.02 | |
| | | | | | | [M + H]^{+ (1)} | |
| 134 | NO₂ | H | H | CH₃ | CF₃ | 6.327 min | E |
| | | | | | | m/z = 434.05 | |
| | | | | | | [M + H]^{+ (1)} | |
| 135 | 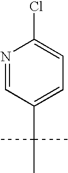 | H | H | CH₃ | CH₃ | 3.418 min m/z = 445.7 [M]⁺ 72° C. | Z |
| 136 | CN | H | H | 2-propynyl | CH₃ | 3.197 min | Z |
| | | | | | | m/z = 383.8 | |
| | | | | | | [M]⁺ | |

*)This statement refers to the stereochemistry of the double bond at the piperazine skeleton.
⁽¹⁾HPLC-column: RP-18 column (XTerra MS 5 mm from Waters); mobile phase:acetonitrile + 0.1% formic acid (A)/water + 0.1% formic acid (B), gradient: from 5:95 (A/B) to 100:0 (A/B) in 8 minutes, at room temperature; MS: Quadrupol Electrospray Ionisation, 80 V (positive mode)
Except for the compounds marked **⁾, the compounds are in each case racemic compounds with respect to the stereocenter at the piperazine skeleton. The compounds marked **⁾ are derived from L-phenylalanine and therefore have the S configuration at this stereocenter.

TABLE 2
Compounds of the general formula I in which R⁴ is CH₃
and R⁷ and R⁸ are each hydrogen
(compounds of the formula I.c).

| | | | | | | | | | (I.c) |
|---|---|---|---|---|---|---|---|---|---|
| 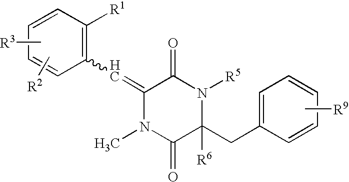 | | | | | | | | | |
| | | | | | | | | RT, [m/z] | |
| | | | | | | | | and/or | Iso- |
| Ex. | R¹ | R² | R³ | R⁵ | R⁶ | R⁹ | R¹⁰ | m.p. | mer*) |
| 137 | CN | H | H | CH₃ | CH₃ | 4-Cl | H | 3.193 min | Z |
| | | | | | | | | m/z = | |
| | | | | | | | | 394.4 | |
| | | | | | | | | [M + H]⁺ | |
| | | | | | | | | 163° C. | |
| 138 | CN | H | H | CH₃ | CH₃ | 4-F | H | 2.934 min | Z |
| | | | | | | | | m/z = | |
| | | | | | | | | 377.9 | |
| | | | | | | | | [M + H]⁺ | |
| 139 | CN | H | H | CH₃ | CH₃ | 2-F | H | 3.055 min | Z |
| | | | | | | | | m/z = | |
| | | | | | | | | 378.1 | |
| | | | | | | | | [M + H]⁺ | |
| | | | | | | | | 175° C. | |
| 140 | CN | H | H | CH₃ | CH₃ | 3-F | H | 3.083 min | Z |
| | | | | | | | | m/z = | |
| | | | | | | | | 378.1 | |
| | | | | | | | | [M + H]⁺ | |
| | | | | | | | | 145° C. | |
| 141 | CN | H | H | CH₃ | CH₃ | 2-Cl | H | 3.182 min | Z |
| | | | | | | | | m/z = | |
| | | | | | | | | 394.1 | |
| | | | | | | | | [M + H]⁺ | |
| | | | | | | | | 176° C. | |
| 142 | CN | H | H | CH₃ | CH₃ | 3-Cl | H | 3.276 min | Z |
| | | | | | | | | m/z = | |
| | | | | | | | | 394.1 | |
| | | | | | | | | [M + H]⁺ | |
| | | | | | | | | 170° C. | |
| 143 | CN | H | H | CH₃ | CH₃ | 2- | H | 3.276 min | Z |
| | | | | | | CH₃ | | m/z = | |
| | | | | | | | | 374.1 | |
| | | | | | | | | [M + H]⁺ | |
| | | | | | | | | 174° C. | |
| 144 | CN | H | H | CH₃ | CH₃ | 3- | H | 3.224 min | Z |
| | | | | | | CH₃ | | m/z = | |
| | | | | | | | | 374.1 | |
| | | | | | | | | [M + H]⁺ | |
| | | | | | | | | 145° C. | |
| 145 | CN | H | H | CH₃ | CH₃ | 4- | H | 3.274 min | Z |
| | | | | | | CH₃ | | m/z = | |
| | | | | | | | | 374.1 | |
| | | | | | | | | [M + H]⁺ | |
| | | | | | | | | 165° C. | |
| 146 | CN | H | H | CH₃ | CH₃ | 2- | H | 3.126 min | Z |
| | | | | | | OCH₃ | | m/z = | |
| | | | | | | | | 390.0 | |
| | | | | | | | | [M + H]⁺ | |
| | | | | | | | | 151° C. | |
| 147 | CN | H | H | CH₃ | CH₃ | 4- | H | 2.963 min | Z |
| | | | | | | OCH₃ | | m/z = | |
| | | | | | | | | 390.1 | |
| | | | | | | | | [M + H]⁺ | |
| | | | | | | | | 123° C. | |
| 148 | CN | H | H | CH₃ | CH₃ | 4-CN | H | 2.863 min | Z |
| | | | | | | | | m/z = | |
| | | | | | | | | 385.1 | |
| | | | | | | | | [M + H]⁺ | |
| | | | | | | | | 203° C. | |
| 149 | J | H | H | CH₃ | CH₃ | 2-F | H | 3.623 min | Z |
| | | | | | | | | m/z = | |
| | | | | | | | | 479.0 | |
| | | | | | | | | [M + H]⁺ | |
| | | | | | | | | 182° C. | |
| 150 | J | H | H | CH₃ | CH₃ | 3-F | H | 3.652 min | Z |
| | | | | | | | | m/z = | |
| | | | | | | | | 479.0 | |
| | | | | | | | | [M + H]⁺ | |
| | | | | | | | | 176° C. | |
| 151 | J | H | H | CH₃ | CH₃ | 2-Cl | H | 3.756 min | Z |
| | | | | | | | | m/z = | |
| | | | | | | | | 495.0 | |
| | | | | | | | | [M + H]⁺ | |
| | | | | | | | | 198° C. | |
| 152 | J | H | H | CH₃ | CH₃ | 3-Cl | H | 3.849 min | Z |
| | | | | | | | | m/z = | |
| | | | | | | | | 495.0 | |
| | | | | | | | | [M + H]⁺ | |
| | | | | | | | | 150° C. | |
| 153 | J | H | H | CH₃ | CH₃ | 2- | H | 3.808 min | Z |
| | | | | | | CH₃ | | m/z = | |
| | | | | | | | | 475.0 | |
| | | | | | | | | [M + H]⁺ | |
| | | | | | | | | 195° C. | |
| 154 | J | H | H | CH₃ | CH₃ | 3- | H | 3.802 min | Z |
| | | | | | | CH₃ | | m/z = | |
| | | | | | | | | 475.0 | |
| | | | | | | | | [M + H]⁺ | |
| | | | | | | | | 79° C. | |
| 155 | J | H | H | CH₃ | CH₃ | 4-CH₃ | H | 3.787 min | Z |
| | | | | | | | | m/z = | |
| | | | | | | | | 475.0 | |
| | | | | | | | | [M + H]⁺ | |
| | | | | | | | | 142° C. | |
| 156 | J | H | H | CH₃ | CH₃ | 4- | H | 3.529 min | Z |
| | | | | | | OCH₃ | | m/z = | |
| | | | | | | | | 491.0 | |
| | | | | | | | | [M + H]⁺ | |
| 157 | J | H | H | CH₃ | CH₃ | 4- | H | 3.350 min | Z |
| | | | | | | CN | | m/z = | |
| | | | | | | | | 486.0 | |
| | | | | | | | | [M + H]⁺ | |
| 158 | NO₂ | H | H | CH₃ | CH₃ | 3-F | H | 3.131 min | E/Z |
| | | | | | | | | m/z = | 1:1 |
| | | | | | | | | 398 | |
| | | | | | | | | [M + H]⁺ | |
| 159 | NO₂ | H | H | CH₃ | CH₃ | 3-F | 5-F | 2.934 min | E/Z |
| | | | | | | | | m/z = | 1:1 |
| | | | | | | | | 377.9 | |
| | | | | | | | | [M + H]⁺ | |
| 160 | J | H | H | CH₃ | CH₃ | 4-F | 5-F | 3.694 min | Z |
| | | | | | | | | m/z = | |
| | | | | | | | | 496.3 | |
| | | | | | | | | [M + H]⁺ | |
| 161 | J | H | H | CH₃ | CH₃ | 3- | H | 3.594 min | Z |
| | | | | | | OCH₃ | | m/z = | |
| | | | | | | | | 490.3 | |
| | | | | | | | | [M + H]⁺ | |
| 162 | J | H | H | CH₃ | CH₃ | 2- | H | 3.415 min | Z |
| | | | | | | CN | | m/z = | |
| | | | | | | | | 485.3 | |
| | | | | | | | | [M + H]⁺ | |
| 163 | J | H | H | CH₃ | CH₃ | 3- | H | 3.404 min | Z |
| | | | | | | CN | | m/z = | |
| | | | | | | | | 485.3 | |
| | | | | | | | | [M + H]⁺ | |
| 164 | J | H | H | CH₃ | CH₃ | 3-F | 5-F | 3.747 min | Z |
| | | | | | | | | m/z = | |
| | | | | | | | | 496.3 | |
| | | | | | | | | [M + H]⁺ | |
| 165 | J | H | H | CH₃ | CH₃ | 3- | H | 3.553 min | Z |
| | | | | | | NO₂ | | m/z = | |
| | | | | | | | | 505.3 | |
| | | | | | | | | [M + H]⁺ | |
| 166 | J | H | H | CH₃ | CH₃ | 3- | 4-F | 3.800 min | Z |
| | | | | | | CH₃ | | m/z = | |
| | | | | | | | | 492.3 | |
| | | | | | | | | [M + H]⁺ | |
| 167 | Br | H | H | CH₃ | CH₃ | 2-F | 3-F | 3.548 min | Z |
| | | | | | | | | m/z = | |
| | | | | | | | | 449.3 | |
| | | | | | | | | [M + H]⁺ | |
| | | | | | | | | 116° C. | |
| 168 | Br | H | H | CH₃ | CH₃ | 2-F | 5-F | 3.550 min | Z |
| | | | | | | | | m/z = | |
| | | | | | | | | 449.3 | |
| | | | | | | | | [M + H]⁺ | |
| | | | | | | | | 116° C. | |
| 169 | Br | H | H | CH₃ | CH₃ | 2-F | 6-F | 3.526 min | Z |
| | | | | | | | | m/z = | |
| | | | | | | | | 449.3 | |
| | | | | | | | | [M + H]⁺ | |
| | | | | | | | | 184° C. | |
| 170 | CN | H | H | CH₃ | CH₃ | 2-F | 3-F | 3.184 min | Z |
| | | | | | | | | m/z = | |
| | | | | | | | | 395.4 | |
| | | | | | | | | [M + H]⁺ | |
| | | | | | | | | 163° C. | |
| 171 | CN | H | H | CH₃ | CH₃ | 2-F | 5-F | 3.181 min | Z |
| | | | | | | | | m/z = | |
| | | | | | | | | 395.4 | |
| | | | | | | | | [M + H]⁺ | |
| | | | | | | | | 178° C. | |
| 172 | CN | H | H | CH₃ | CH₃ | 2-F | 6-F | 3.152 min | Z |
| | | | | | | | | m/z = | |
| | | | | | | | | 395.4 | |
| | | | | | | | | [M + H]⁺ | |
| | | | | | | | | 168° C. | |
| 173 | Br | H | H | CH₃ | CH₃ | 2- | H | 3.580 min | Z |
| | | | | | | OCHF₂ | | m/z = | |
| | | | | | | | | 479.3 | |
| | | | | | | | | [M + H]⁺ | |
| 174 | Br | H | H | CH₃ | CH₃ | 3- | H | 3.687 min | Z |
| | | | | | | OCHF₂ | | m/z = | |
| | | | | | | | | 479.3 | |
| | | | | | | | | [M + H]⁺ | |
| 175 | CN | H | H | CH₃ | CH₃ | 2- | H | 3.194 min | Z |
| | | | | | | OCHF₂ | | m/z = | |
| | | | | | | | | 425.4 | |
| | | | | | | | | [M + H]⁺ | |
| 176 | CN | H | H | CH₃ | CH₃ | 3- | H | 3.808 min | Z |
| | | | | | | OCHF₂ | | m/z = | |
| | | | | | | | | 425.4 | |
| | | | | | | | | [M + H]⁺ | |
| 177 | CN | H | H | CH₃ | CH₃ | 3- | H | 3.403 min | Z |
| | | | | | | CF₃ | | m/z = | |
| | | | | | | | | 427.4 | |
| | | | | | | | | [M + H]⁺ | |
| 178 | CN | H | H | CH₃ | CH₃ | 3-CF₃ | H | 3.397 min | E |
| | | | | | | | | m/z = | |
| | | | | | | | | 427.4 | |
| | | | | | | | | [M + H]⁺ | |
| 179 | NO₂ | H | H | CH₃ | CH₃ | 3-F | H | 3.095 min | E |
| | | | | | | | | m/z = | |
| | | | | | | | | 397.4 | |
| | | | | | | | | [M + H]⁺ | |
| 180 | CN | H | H | CH₃ | CH₃ | 2- | H | 2.941 min | Z |
| | | | | | | CN | | m/z = | |
| | | | | | | | | 384.4 | |
| | | | | | | | | [M + H]⁺ | |
| | | | | | | | | 153° C. | |
| 181 | CN | H | H | CH₃ | CH₃ | 3- | H | 2.933 min | Z |
| | | | | | | CN | | m/z = | |
| | | | | | | | | 384.4 | |
| | | | | | | | | [M + H]⁺ | |
| | | | | | | | | 184° C. | |
| 182 | CN | H | H | CH₃ | CH₃ | 3-F | 5-F | 3.252 min | Z |
| | | | | | | | | m/z = | |
| | | | | | | | | 395.4 | |
| | | | | | | | | [M + H]⁺ | |
| | | | | | | | | 170° C. | |
| 183 | CN | H | H | CH₃ | CH₃ | 3- | H | 3.086 min | Z |
| | | | | | | NO₂ | | m/z = | |
| | | | | | | | | 404.4 | |
| | | | | | | | | [M + H]⁺ | |
| | | | | | | | | 154° C. | |
| 184 | CN | H | H | CH₃ | CH₃ | 3- | 4-F | 3.290 min | Z |
| | | | | | | CH₃ | | m/z = | |
| | | | | | | | | 391.4 | |
| | | | | | | | | [M + H]⁺ | |
| | | | | | | | | 174° C. | |
| 185 | J | H | H | CH₃ | CH₃ | 3- | 4-F | 3.800 min | Z |
| | | | | | | CH₃ | | m/z = | |
| | | | | | | | | 492.3 | |
| | | | | | | | | [M + H]⁺ | |
| 186 | CN | H | H | CH₃ | CH₃ | 3- | 4-F | 3.094 min | Z/E |
| | | | | | | OCH₃ | | m/z = | |
| | | | | | | | | 407.4 | |
| | | | | | | | | [M]⁺ | |
| | | | | | | | | 119° C. | |

*)This statement refers to the stereochemistry of the double bond at the piperazine skeleton. The compounds prepared are in each case racemates.

TABLE 3

| | |
|---|---|
| Compounds of the general formula I in which R⁷, R⁸, R⁹ and | a |

R¹⁰ are each hydrogen (compounds of the formula I.d).

| | | | | | | | | (I.c) |
|---|---|---|---|---|---|---|---|---|
| 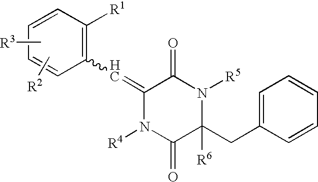 | | | | | | | | |
| | | | | | | | RT, [m/z] | |
| | | | | | | | and/or | Iso- |
| Ex. | R¹ | R² | R³ | R⁴ | R⁵ | R⁶ | m.p. | mer*) |
| 187 | CN | H | H | ethyl | H | CH₃ | 3.069 min | Z |
| | | | | | | | m/z = | |
| | | | | | | | 360.1 | |
| | | | | | | | [M + H]⁺ | |
| | | | | | | | 182° C. | |
| 188 | CN | H | H | ethyl | CH₃ | CH₃ | 3.334 min | Z |
| | | | | | | | m/z = | |
| | | | | | | | 374.1 | |
| | | | | | | | [M + H]⁺ | |
| | | | | | | | 103° C. | |
| 189 | NO₂ | H | H | CH₂CH═CH₂ | CH₃ | CH₃ | 3.290 min | Z |
| | | | | | | | m/z = | |
| | | | | | | | 406 | |
| | | | | | | | [M + H]⁺ | |
| | | | | | | | 118° C. | |
| 190 | NO₂ | H | H | 2-propynyl | CH₃ | CH₃ | 3.270 min | Z |
| | | | | | | | m/z = | |
| | | | | | | | 404.1 | |
| | | | | | | | [M + H]⁺ | |

*)This statement refers to the stereochemistry of the double bond at the piperazine skeleton. The compounds prepared are in each case racemates.

### II: Use Examples

The herbicidal activity of the compounds of the formula I was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted. This cover caused uniform germination of the test plants, unless this has been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A good herbicidal activity is given at values of at least 70 and a very good herbicidal activity is given at values of at least 85.

The plants used in the greenhouse experiments belonged to the following species:

| Bayer Code | Scientific name | Common name |
|---|---|---|
| AMARE | Amaranthus retoflexus | redroot pigweed |
| APESV | Apera spica-venti | windgrass |
| CHEAL | Chenopodium album | common lambsquarters |
| ECHCG | Echinochloa crus-galli | barnyard grass |
| GALAP | Galium aparine | catchweed bedstraw |
| LOLMU | Lolium multiflorum | Italian ryegrass |
| SETVI | Setaria viridis | green foxtail |

The compounds of Examples 3, 6, 7, 11, 12, 13, 16, 18, 24, 26, 39, 43, 44, 47, 55, 56 and 138, applied by the post-emergence method at an application rate of 0.5 kg/ha, showed good to very good herbicidal activity against AMARE.

The compound of Example 43, applied by the post-emergence method at an application rate of 0.5 kg/ha, showed good herbicidal activity against APESV.

The compounds of Examples 3, 7, 11, 12, 13, 14, 16, 18, 25, 39 and 55, applied by the post-emergence method at an application rate of 0.5 kg/ha, showed good to very good herbicidal activity against CHEAL.

The compounds of Examples 44 and 47, applied by the post-emergence method at an application rate of 0.5 kg/ha, showed very good herbicidal activity against ECHCG.

The compound of Example 137, applied by the post-emergence method at an application rate of 0.5 kg/ha, showed good herbicidal activity against GALAP.

The compound of Example 10, applied by the post-emergence method at an application rate of 0.5 kg/ha, showed good herbicidal activity against LOLMU.

The compounds of Examples 6, 7, 10, 11, 12, 13, 14, 16, 18, 25, 26, 29, 39, 44, 47, 55, 56 and 138, applied by the post-emergence method at an application rate of 0.5 kg/ha, showed good to very good herbicidal activity against SETVI.

The compounds of Examples 24, 29, 43, and 137, applied by the pre-emergence method at an application rate of 0.5 kg/ha, showed very good herbicidal activity against APESV.

The compound of Example 25, applied by the pre-emergence method at an application rate of 0.5 kg/ha, showed good herbicidal activity against CHEAL.

The compound of Example 25, applied by the pre-emergence method at an application rate of 0.5 kg/ha, showed good herbicidal activity against SETVI.

1-29. (canceled)

30. A piperazine compound of the formula I 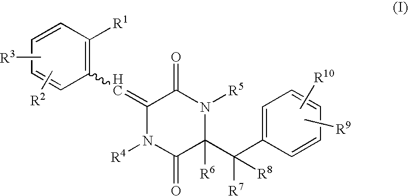
in which
R¹ is selected from the group consisting of halogen, cyano, nitro, Z—C(═O)—R¹¹, phenyl and a 5- or 6-membered heterocyclic radical which has 1, 2, 3 or 4 heteroatoms selected from the group consisting of O, N and S as ring atoms, where phenyl and the heterocyclic radical are unsubstituted or may have 1, 2, 3 or 4 substituents R^{1a} independently of one another selected from the group consisting of halogen, CN, NO₂, C₁-C₄-alkyl, C₁-C₄-haloalkyl, C₁-C₄-alkoxy and C₁-C₄-haloalkoxy, and in which
Z is a covalent bond or a CH₂ group;
R¹¹ is hydrogen, C₁-C₆-alkyl, C₃-C₆-cycloalkyl, C₂-C₆-alkenyl, C₅-C₆-cycloalkenyl, C₂-C₆-alkynyl, hydroxyl, C₁-C₆-alkoxy, C₃-C₆-alkenyloxy, C₃-C₆-alkynyl oxy, amino, C₁-C₆-alkylamino, [di-(C₁-C₆)-alkyl]amino, C₁-C₆-alkoxyamino, C₁-C₆-alkylsulfonylamino, C₁-C₆-alkylaminosulfonylamino, (di-(C₁-C₆)-alkylamino)sulfonylamino, C₃-C₆-alkenylamino, C₃-C₆-alkynylamino, N—(C₂-C₆-alkenyl)-N—(C₁-C₆-alkyl)-amino, N—(C₂-C₆-alkynyl)-N—(C₁-C₆-alkyl)-amino, N—(C₁-C₆-alkoxy)-N—(C₁-C₆-alkyl)-amino, N—(C₂-C₆-alkenyl)-N—(C₁-C₆-alkoxy)-amino, N—(C₂-C₆-alkynyl)-N—(C₁-C₆-alkoxy)-amino, phenyl, phenoxy or phenylamino;
where the alkyl moieties in the radicals listed under R¹¹ may be partially or fully halogenated and the phenyl moieties in the radicals listed under R¹¹ may carry 1, 2, 3 or 4 substituents R^{11a} selected from the group consisting of halogen, CN, NO₂, C₁-C₄-alkyl, C₁-C₄-haloalkyl, C₁-C₄-alkoxy and C₁-C₄-haloalkoxy;
R² is hydrogen, halogen, cyano, nitro, C₁-C₄-alkyl, C₁-C₄-haloalkyl, C₂-C₄-alkenyl, C₁-C₄-alkoxy, C₁-C₄-haloalkoxy, benzyl or a group S(O)ₙR²¹ in which R²¹ is C₁-C₄-alkyl or C₁-C₄-haloalkyl and n is 0, 1 or 2;
R³ is hydrogen or halogen;
R⁴ is C₁-C₄-alkyl, C₃-C₄-alkenyl or C₃-C₄-alkynyl;
R⁵ is hydrogen, C₁-C₄-alkyl, C₃-C₄-alkenyl, C₃-C₄-alkynyl or a group C(═O)R⁵¹ in which R⁵¹ is hydrogen, C₁-C₄-alkyl, C₁-C₄-haloalkyl, C₁-C₄-alkoxy or C₁-C₄-haloalkoxy;
R⁶ is C₁-C₄-alkyl, C₁-C₄-hydroxyalkyl or C₁-C₄-haloalkyl;
R⁷, R⁸ independently of one another are hydrogen, OH, C₁-C₄-alkoxy, C₁-C₄-haloalkyloxy, C₁-C₄-alkyl or C₁-C₄-haloalkyl;
R⁹, R¹⁰ independently of one another are selected from the group consisting of hydrogen, halogen, CN, NO₂, C₁-C₄-alkyl, C₁-C₄-haloalkyl, C₂-C₄-alkenyl, C₁-Cₐ-alkoxy and C₁-Cₐ-haloalkoxy;
or an agriculturally useful salt of this compound.

31. The piperazine compound according to claim 30 in which R¹ is cyano, nitro or a 5- or 6-membered heteroaromatic radical which has either 1, 2 or 3 nitrogen atoms or 1 oxygen or 1 sulfur atom and, if appropriate, 1 or 2 nitrogen atoms as ring members and which is unsubstituted or may have 1 or 2 substituents selected from R^{1a}.

32. The piperazine compound according to claim 30 in which R¹ is halogen, in particular chlorine or bromine.

33. The piperazine compound according to claim 30 in which R⁴ is methyl.

34. The piperazine compound according to claim 30 in which R⁵ is hydrogen, methyl or ethyl.

35. The piperazine compound according to claim 30 in which R⁵ is C(═O)R⁵¹ in which R⁵¹ is hydrogen, C₁-C₄-alkyl or C₁-C₄-haloalkyl.

36. The piperazine compound according to claim 30 in which R⁶ is methyl or ethyl.

37. The piperazine compound according to claim 30 in which R⁷ and R⁸ are hydrogen.

38. The piperazine compound according to claim 30 in which R¹⁰ is hydrogen.

39. The piperazine compound according claim 30 having the general formula Ia: 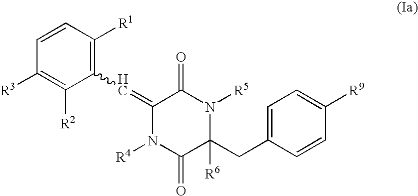
in which
R¹ is cyano or nitro;
R² is hydrogen, fluorine, chlorine, C₁-C₂-alkyl, ethenyl or C₁-C₂-alkoxy;
R³ is fluorine or hydrogen;
R⁴ is methyl;
R⁵ is hydrogen, methyl or ethyl;
R⁶ is methyl or ethyl; and
R⁹ is hydrogen or halogen,
or an agriculturally useful salt of this compound.

40. The piperazine compound according to claim 30, selected from the group consisting of:
2-[5-benzyl-1,4,5-trimethyl-3,6-dioxopiperazin-2-ylidenemethyl]benzonitrile,
2-[5-benzyl-1,4,5-trimethyl-3,6-dioxopiperazin-2-ylidenemethyl]-3-fluorobenzonitrile,
2-[5-benzyl-1,4,5-trimethyl-3,6-dioxopiperazin-2-ylidenemethyl]-3-methoxybenzonitrile,
2-[5-benzyl-1,4,5-trimethyl-3,6-dioxopiperazin-2-ylidenemethyl]-3,4-difluorobenzonitrile,
2-[5-benzyl-1,4,5-trimethyl-3,6-dioxopiperazin-2-ylidenemethyl]-3-methylbenzonitrile,
2-[5-benzyl-1,4,5-trimethyl-3,6-dioxopiperazin-2-ylidenemethyl]-3-ethenylbenzonitrile,
2-[5-benzyl-1,5-dimethyl-3,6-dioxopiperazin-2-ylidenemethyl]benzonitrile,
2-[5-benzyl-1,5-dimethyl-3,6-dioxopiperazin-2-ylidenemethyl]-3-fluorobenzonitrile,
2-[5-benzyl-1,5-dimethyl-3,6-dioxopiperazin-2-ylidenemethyl]-3-methoxybenzonitrile,
2-[5-benzyl-1,5-dimethyl-3,6-dioxopiperazin-2-ylidenemethyl]-3,4-difluorobenzonitrile,
2-[5-benzyl-1,5-dimethyl-3,6-dioxopiperazin-2-ylidenemethyl]-3-methylbenzonitrile,
2-[5-benzyl-1,5-dimethyl-3,6-dioxopiperazin-2-ylidenemethyl]-3-ethenylbenzonitrile,
2-[5-benzyl-5-ethyl-1,4-dimethyl-3,6-dioxopiperazin-2-ylidenemethyl]benzonitrile,
2-[5-benzyl-5-ethyl-1,4-dimethyl-3,6-dioxopiperazin-2-ylidenemethyl]-3-fluorobenzonitrile,
2-[5-benzyl-5-ethyl-1,4-dimethyl-3,6-dioxopiperazin-2-ylidenemethyl]-3-methoxybenzonitrile,
2-[5-benzyl-5-ethyl-1,4-dimethyl-3,6-dioxopiperazin-2-ylidenemethyl]-3,4-difluorobenzonitrile,
2-[5-benzyl-5-ethyl-1,4-dimethyl-3,6-dioxopiperazin-2-ylidenemethyl]-3-methylbenzonitrile,
2-[5-benzyl-5-ethyl-1,4-dimethyl-3,6-dioxopiperazin-2-ylidenemethyl]-3-ethenylbenzonitrile,
2-[5-benzyl-5-ethyl-1-methyl-3,6-dioxopiperazin-2-ylidenemethyl]benzonitrile,
2-[5-benzyl-5-ethyl-1-methyl-3,6-dioxopiperazin-2-ylidenemethyl]-3-fluorobenzonitrile,
2-[5-benzyl-5-ethyl-1-methyl-3,6-dioxopiperazin-2-ylidenemethyl]-3-methoxybenzonitrile,
2-[5-benzyl-5-ethyl-1-methyl-3,6-dioxopiperazin-2-ylidenemethyl]-3,4-difluorobenzonitrile,
2-[5-benzyl-5-ethyl-1-methyl-3,6-dioxopiperazin-2-ylidenemethyl]-3-methylbenzonitrile,
2-[5-benzyl-5-ethyl-1-methyl-3,6-dioxopiperazin-2-ylidenemethyl]-3-ethenylbenzonitrile,
3-benzyl-6-[1-(2-nitrophenyl)methylidene]-1,3,4-trimethylpiperazine-2,5-dione,
3-benzyl-6-[1-(2-fluoro-6-nitrophenyl)methylidene]-1,3,4-trimethylpiperazine-2,5-dione,
3-benzyl-6-[1-(2,3-difluoro-6-nitrophenyl)methylidene]-1,3,4-trimethylpiperazine-2,5-dione,
3-benzyl-6-[1-(2-methoxy-6-nitrophenyl)methylidene]-1,3,4-trimethylpiperazine-2,5-dione,
3-benzyl-6-[1-(2-methyl-6-nitrophenyl)methylidene]-1,3,4-trimethylpiperazine-2,5-dione,
3-benzyl-6-[1-(2-ethenyl-6-nitrophenyl)methylidene]-1,3,4-trimethylpiperazine-2,5-dione,
3-benzyl-6-[1-(2-nitrophenyl)methylidene]-1,3-dimethylpiperazine-2,5-dione,
3-benzyl-6-[1-(2-fluoro-6-nitrophenyl)methylidene]-1,3-dimethylpiperazine-2,5-dione,
3-benzyl-6-[1-(2,3-difluoro-6-nitrophenyl)methylidene]-1,3-dimethylpiperazine-2,5-dione,
3-benzyl-6-[1-(2-methoxy-6-nitrophenyl)methylidene]-1,3-dimethylpiperazine-2,5-dione,
3-benzyl-6-[1-(2-methyl-6-nitrophenyl)methylidene]-1,3-dimethylpiperazine-2,5-dione,
3-benzyl-6-[1-(2-ethenyl-6-nitrophenyl)methylidene]-1,3-dimethylpiperazine-2,5-dione,
3-benzyl-6-[1-(2-nitrophenyl)methylidene]-3-ethyl-1,4-dimethylpiperazine-2,5-dione,
3-benzyl-6-[1-(2-fluoro-6-nitrophenyl)methylidene]-3-ethyl-1,4-dimethylpiperazine-2,5-dione,
3-benzyl-6-[1-(2,3-difluoro-6-nitrophenyl)methylidene]-3-ethyl-1,4-dimethylpiperazine-2,5-dione,
3-benzyl-6-[1-(2-methoxy-6-nitrophenyl)methylidene]-3-ethyl-1,4-dimethylpiperazine-2,5-dione,
3-benzyl-6-[1-(2-methyl-6-nitrophenyl)methylidene]-3-ethyl-1,4-dimethylpiperazine-2,5-dione,
3-benzyl-6-[1-(2-ethenyl-6-nitrophenyl)methylidene]-3-ethyl-1,4-dimethylpiperazine-2,5-dione,
3-benzyl-6-[1-(2-nitrophenyl)methylidene]-3-ethyl-1-methylpiperazine-2,5-dione,
3-benzyl-6-[1-(2-fluoro-6-nitrophenyl)methylidene]-3-ethyl-1-methylpiperazine-2,5-dione,
3-benzyl-6-[1-(2,3-difluoro-6-nitrophenyl)methylidene]-3-ethyl-1-methylpiperazine-2,5-dione,
3-benzyl-6-[1-(2-methoxy-6-nitrophenyl)methylidene]-3-ethyl-1-methylpiperazine-2,5-dione,
3-benzyl-6-[1-(2-methyl-6-nitrophenyl)methylidene]-3-ethyl-1-methylpiperazine-2,5-dione, and
3-benzyl-6-[1-(2-ethenyl-6-nitrophenyl)methylidene]-3-ethyl-1-methylpiperazine-2,5-dione,
2-[5-(4-fluorobenzyl)-1,4,5-trimethyl-3,6-dioxopiperazin-2-ylidenemethyl]-benzonitrile,
2-[5-(4-fluorobenzyl)-1,4,5-trimethyl-3,6-dioxopiperazin-2-ylidenemethyl]-3-fluorobenzonitrile,
2-[5-(4-fluorobenzyl)-1,4,5-trimethyl-3,6-dioxopiperazin-2-ylidenemethyl]-3-methoxybenzonitrile,
2-[5-(4-fluorobenzyl)-1,4,5-trimethyl-3,6-dioxopiperazin-2-ylidenemethyl]-3,4-difluorobenzonitrile,
2-[5-(4-fluorobenzyl)-1,4,5-trimethyl-3,6-dioxopiperazin-2-ylidenemethyl]-3-methylbenzonitrile,
2-[5-(4-fluorobenzyl)-1,4,5-trimethyl-3,6-dioxopiperazin-2-ylidenemethyl]-3-ethenylbenzonitrile,
2-[5-(4-fluorobenzyl)-1,5-dimethyl-3,6-dioxopiperazin-2-ylidenemethyl]-benzonitrile,
2-[5-(4-fluorobenzyl)-1,5-dimethyl-3,6-dioxopiperazin-2-ylidenemethyl]-3-fluorobenzonitrile,
2-[5-(4-fluorobenzyl)-1,5-dimethyl-3,6-dioxopiperazin-2-ylidenemethyl]-3-methoxybenzonitrile,
2-[5-(4-fluorobenzyl)-1,5-dimethyl-3,6-dioxopiperazin-2-ylidenemethyl]-3,4-difluorobenzonitrile,
2-[5-(4-fluorobenzyl)-1,5-dimethyl-3,6-dioxopiperazin-2-ylidenemethyl]-3-methylbenzonitrile,
2-[5-(4-fluorobenzyl)-1,5-dimethyl-3,6-dioxopiperazin-2-ylidenemethyl]-3-ethenylbenzonitrile,
2-[5-(4-fluorobenzyl)-5-ethyl-1,4-dimethyl-3,6-dioxopiperazin-2-ylidenemethyl]-benzonitrile,
2-[5-(4-fluorobenzyl)-5-ethyl-1,4-dimethyl-3,6-dioxopiperazin-2-ylidenemethyl]-3-fluorobenzonitrile,
2-[5-(4-fluorobenzyl)-5-ethyl-1,4-dimethyl-3,6-dioxopiperazin-2-ylidenemethyl]-3-methoxybenzonitrile,
2-[5-(4-fluorobenzyl)-5-ethyl-1,4-dimethyl-3,6-dioxopiperazin-2-ylidenemethyl]-3,4-difluorobenzonitrile,
2-[5-(4-fluorobenzyl)-5-ethyl-1,4-dimethyl-3,6-dioxopiperazin-2-ylidenemethyl]-3-methylbenzonitrile,
2-[5-(4-fluorobenzyl)-5-ethyl-1,4-dimethyl-3,6-dioxopiperazin-2-ylidenemethyl]-3-ethenylbenzonitrile,
2-[5-(4-fluorobenzyl)-5-ethyl-1-methyl-3,6-dioxopiperazin-2-ylidenemethyl]-benzonitrile,
2-[5-(4-fluorobenzyl)-5-ethyl-1-methyl-3,6-dioxopiperazin-2-ylidenemethyl]-3-fluorobenzonitrile,
2-[5-(4-fluorobenzyl)-5-ethyl-1-methyl-3,6-dioxopiperazin-2-ylidenemethyl]-3-methoxybenzonitrile,
2-[5-(4-fluorobenzyl)-5-ethyl-1-methyl-3,6-dioxopiperazin-2-ylidenemethyl]-3,4-difluorobenzonitrile,
2-[5-(4-fluorobenzyl)-5-ethyl-1-methyl-3,6-dioxopiperazin-2-ylidenemethyl]-3-methylbenzonitrile,
2-[5-(4-fluorobenzyl)-5-ethyl-1-methyl-3,6-dioxopiperazin-2-ylidenemethyl]-3-ethenylbenzonitrile,
3-(4-fluorobenzyl)-6-[1-(2-nitrophenyl)methylidene]-1,3,4-trimethylpiperazine-2,5-dione,
3-(4-fluorobenzyl)-6-[1-(2-fluoro-6-nitrophenyl)methylidene]-1,3,4-trimethylpiperazine-2,5-dione,
3-(4-fluorobenzyl)-6-[1-(2,3-difluoro-6-nitrophenyl)methylidene]-1,3,4-trimethylpiperazine-2,5-dione,
3-(4-fluorobenzyl)-6-[1-(2-methoxy-6-nitrophenyl)methylidene]-1,3,4-trimethylpiperazine-2,5-dione,
3-(4-fluorobenzyl)-6-[1-(2-methyl-6-nitrophenyl)methylidene]-1,3,4-trimethylpiperazine-2,5-dione,
3-(4-fluorobenzyl)-6-[1-(2-ethenyl-6-nitrophenyl)methylidene]-1,3,4-trimethylpiperazine-2,5-dione,
3-(4-fluorobenzyl)-6-[1-(2-nitrophenyl)methylidene]-1,3-dimethylpiperazine-2,5-dione,
3-(4-fluorobenzyl)-6-[1-(2-fluoro-6-nitrophenyl)methylidene]-1,3-dimethylpiperazine-2,5-dione,
3-(4-fluorobenzyl)-6-[1-(2,3-difluoro-6-nitrophenyl)methylidene]-1,3-dimethylpiperazine-2,5-dione,
3-(4-fluorobenzyl)-6-[1-(2-methoxy-6-nitrophenyl)methylidene]-1,3-dimethylpiperazine-2,5-dione,
3-(4-fluorobenzyl)-6-[1-(2-methyl-6-nitrophenyl)methylidene]-1,3-dimethylpiperazine-2,5-dione,
3-(4-fluorobenzyl)-6-[1-(2-ethenyl-6-nitrophenyl)methylidene]-1,3-dimethylpiperazine-2,5-dione,
3-(4-fluorobenzyl)-6-[1-(2-nitrophenyl)methylidene]-3-ethyl-1,4-dimethylpiperazine-2,5-dione,
3-(4-fluorobenzyl)-6-[1-(2-fluoro-6-nitrophenyl)methylidene]-3-ethyl-1,4-dimethylpiperazine-2,5-dione,
3-(4-fluorobenzyl)-6-[1-(2,3-difluoro-6-nitrophenyl)methylidene]-3-ethyl-1,4-dimethylpiperazine-2,5-dione,
3-(4-fluorobenzyl)-6-[1-(2-methoxy-6-nitrophenyl)methylidene]-3-ethyl-1,4-dimethylpiperazine-2,5-dione,
3-(4-fluorobenzyl)-6-[1-(2-methyl-6-nitrophenyl)methylidene]-3-ethyl-1,4-dimethylpiperazine-2,5-dione,
3-(4-fluorobenzyl)-6-[1-(2-ethenyl-6-nitrophenyl)methylidene]-3-ethyl-1,4-dimethylpiperazine-2,5-dione,
3-(4-fluorobenzyl)-6-[1-(2-nitrophenyl)methylidene]-3-ethyl-1-methylpiperazine-2,5-dione,
3-(4-fluorobenzyl)-6-[1-(2-fluoro-6-nitrophenyl)methylidene]-3-ethyl-1-methylpiperazine-2,5-dione,
3-(4-fluorobenzyl)-6-[1-(2,3-difluoro-6-nitrophenyl)methylidene]-3-ethyl-1-methylpiperazine-2,5-dione,
3-(4-fluorobenzyl)-6-[1-(2-methoxy-6-nitrophenyl)methylidene]-3-ethyl-1-methylpiperazine-2,5-dione,
3-(4-fluorobenzyl)-6-[1-(2-methyl-6-nitrophenyl)methylidene]-3-ethyl-1-methylpiperazine-2,5-dione,
3-(4-fluorobenzyl)-6-[1-(2-ethenyl-6-nitrophenyl)methylidene]-3-ethyl-1-methylpiperazine-2,5-dione,
2-[5-benzyl-1,4,5-trimethyl-3,6-dioxopiperazin-2-ylidenemethyl]-3-bromobenzonitrile,
2-[5-benzyl-1,4,5-trimethyl-3,6-dioxopiperazin-2-ylidenemethyl]isophthalonitrile,
3-benzyl-6-[1-(2,3-difluoro-6-nitrophenyl)methylidene]-1,3,4-trimethylpiperazine-2,5-dione,
3-benzyl-6-[1-(2-nitro-5-methoxyphenyl)methylidene]-1,3,4-trimethylpiperazine-2,5-dione,
2-[5-benzyl-1,4,5-trimethyl-3,6-dioxopiperazin-2-ylidenemethyl]-3-nitrobenzonitrile,
3-benzyl-6-[1-(2-ethenyl-6-nitrophenyl)methylidene]-1,3,4-trimethylpiperazine-2,5-dione,
3-benzyl-6-[1-(3-chloro-2-nitrophenyl)methylidene]-1,3,4-trimethylpiperazine-2,5-dione,
3-benzyl-6-[1-(2-nitro-6-trifluoromethylphenyl)methylidene]-1,3,4-trimethylpiperazine-2,5-dione,
2-[5-benzyl-1,5-dimethyl-3,6-dioxopiperazin-2-ylidenemethyl]benzonitrile,
3-benzyl-6-[1-(2-methoxy-6-nitrophenyl)methylidene]-1,3,4-trimethylpiperazine-2,5-dione,
2-[5-benzyl-1,4,5-trimethyl-3,6-dioxopiperazin-2-ylidenemethyl]-4-fluorobenzonitrile,
2-[5-benzyl-1,4,5-trimethyl-3,6-dioxopiperazin-2-ylidenemethyl]-5-methylbenzonitrile,
2-[5-benzyl-1,4,5-trimethyl-3,6-dioxopiperazin-2-ylidenemethyl]-6-fluorobenzonitrile,
3-benzyl-1,3,4-trimethyl-6-[2-(1-methyl-1H-pyrrol-2-yl)benzylidene]piperazine-2,5-dione,
3-benzyl-6-(2-furan-2-yl-benzylidene)-1,3,4-trimethylpiperazine-2,5-dione,
2-[5-benzyl-5-fluoromethyl-1,4-dimethyl-3,6-dioxopiperazin-2-ylidenemethyl]-benzonitrile,
3-benzyl-1,3,4-trimethyl-6-(4-methyl-2-nitrobenzylidene)piperazine-2,5-dione,
2-[5-benzyl-1,4,5-trimethyl-3,6-dioxopiperazin-2-ylidenemethyl]-4-methoxybenzonitrile,
3-benzyl-6-[2-(2-chloropyrimidin-5-yl)benzylidene]-1,3,4-trimethylpiperazine-2,5-dione,
3-benzyl-6-[2-(6-fluoropyridin-2-yl)benzylidene]-1,3,4-trimethylpiperazine-2,5-dione,
2-[5-benzyl-1,4,5-trimethyl-3,6-dioxopiperazin-2-ylidenemethyl]-4-fluorobenzonitrile,
2-[5-benzyl-1,4,5-trimethyl-3,6-dioxopiperazin-2-ylidenemethyl]-4-trifluoromethylbenzonitrile,
3-benzyl-1,3,4-trimethyl-6-[2-(1-methyl-1H-imidazol-2-yl)benzylidene]piperazine-2,5-dione,
3-benzyl-3-fluoromethyl-1,4-dimethyl-6-(2-nitrobenzylidene)piperazine-2,5-dione,
3-benzyl-6-(5-bromo-2-nitrobenzylidene)-1,3,4-trimethylpiperazine-2,5-dione,
2-[5-benzyl-1,4,5-trimethyl-3,6-dioxopiperazin-2-ylidenemethyl]-4-difluoromethoxybenzonitrile,
2-[5-benzyl-1,4,5-trimethyl-3,6-dioxopiperazin-2-ylidenemethyl]-4-methanesulfonyl-benzonitrile,
2-[5-benzyl-1,4,5-trimethyl-3,6-dioxopiperazin-2-ylidenemethyl]-4-methanesulfinyl-benzonitrile,
2-[5-benzyl-1,4,5-trimethyl-3,6-dioxopiperazin-2-ylidenemethyl]-4-methylsulfanylbenzonitrile,
2-[5-benzyl-1,4,5-trimethyl-3,6-dioxopiperazin-2-ylidenemethyl]-3-fluoro-4-methoxybenzonitrile,
2-[5-benzyl-1,4,5-trimethyl-3,6-dioxopiperazin-2-ylidenemethyl]-4,6-difluorobenzonitrile,
3-benzyl-1,3,4-trimethyl-6-[2-(2-methyl-2H-pyrazol-3-yl)benzylidene]piperazine-2,5-dione,
3-benzyl-1,3,4-trimethyl-6-[2-(5-methylthiophen-2-yl)benzylidene]piperazine-2,5-dione,
3-benzyl-1,3,4-trimethyl-6-[2-(3-methylthiophen-2-yl)benzylidene]piperazine-2,5-dione,
2-[5-benzyl-4-ethyl-1,5-dimethyl-3,6-dioxopiperazin-2-ylidenemethyl]benzonitrile,
2-[5-benzyl-4-isopropyl-1,5-dimethyl-3,6-dioxopiperazin-2-ylidenemethyl]-benzonitrile,
2-[5-benzyl-4-butyl-1,5-dimethyl-3,6-dioxopiperazin-2-ylidenemethyl]benzonitrile,
2-[4-allyl-5-benzyl-1,5-dimethyl-3,6-dioxopiperazin-2-ylidenemethyl]benzonitrile,
2-[5-benzyl-5-trifluoromethyl-1,4-dimethyl-3,6-dioxopiperazin-2-ylidenemethyl]-benzonitrile,
3-benzyl-6-[1-(2-nitrophenyl)methylidene]-1,4-dimethyl-3-trifluoromethylpiperazine-2,5-dione,
3-benzyl-6-[2-(6-chloropyridin-3-yl)benzylidene]-1,3,4-trimethylpiperazine-2,5-dione,
2-[5-benzyl-1,5-dimethyl-4-prop-2-ynyl-3,6-dioxopiperazin-2-ylidenemethyl]-benzonitrile,
3-(3-fluorobenzyl)-6-[1-(2-nitrophenyl)methylidene]-1,3,4-trimethylpiperazine-2,5-dione,
3-(3,5-difluorobenzyl)-6-[1-(2-nitrophenyl)methylidene]-1,3,4-trimethylpiperazine-2,5-dione,
2-[5-(2,3-difluorobenzyl)-1,4,5-trimethyl-3,6-dioxopiperazin-2-ylidenemethyl]-benzonitrile,
2-[5-(2,5-difluorobenzyl)-1,4,5-trimethyl-3,6-dioxopiperazin-2-ylidenemethyl]-benzonitrile,
2-[5-(2,6-difluorobenzyl)-1,4,5-trimethyl-3,6-dioxopiperazin-2-ylidenemethyl]-benzonitrile,
2-[5-(2-difluoromethoxybenzyl)-1,4,5-trimethyl-3,6-dioxopiperazin-2-ylidenemethyl]benzonitrile,
2-[5-(3-difluoromethoxybenzyl)-1,4,5-trimethyl-3,6-dioxopiperazin-2-ylidenemethyl]benzonitrile,
2-[5-(3-trifluoromethylbenzyl)-1,4,5-trimethyl-3,6-dioxopiperazin-2-ylidenemethyl]benzonitrile,
3-(3-fluorobenzyl)-6-[1-(2-nitrophenyl)methylidene]-1,3,4-trimethylpiperazine-2,5-dione,
2-[5-(2-cyanobenzyl)-1,4,5-trimethyl-3,6-dioxopiperazin-2-ylidenemethyl]-benzonitrile,
2-[5-(3-cyanobenzyl)-1,4,5-trimethyl-3,6-dioxopiperazin-2-ylidenemethyl]-benzonitrile,
2-[5-(3,5-difluorobenzyl)-1,4,5-trimethyl-3,6-dioxopiperazin-2-ylidenemethyl]-benzonitrile,
2-[5-(3-nitrobenzyl)-1,4,5-trimethyl-3,6-dioxopiperazin-2-ylidenemethyl]-benzonitrile,
2-[5-(4-fluoro-3-methylbenzyl)-1,4,5-trimethyl-3,6-dioxopiperazin-2-ylidenemethyl]benzonitrile,
2-[5-(4-fluoro-3-methoxybenzyl)-1,4,5-trimethyl-3,6-dioxopiperazine-2-ylidenemethyl]benzonitrile,
1-allyl-3-benzyl-3,4-dimethyl-6-[1-(2-nitrophenyl)methylidene]piperazine-2,5-dione and
3-benzyl-6-[1-(2-nitrophenyl)methylidene]-1-prop-2-ynyl-3,4-dimethylpiperazine-2,5-dione.

41. The piperazine compound of claim 30 where the exo double bond at the piperazine ring has the (Z) configuration.

42. A composition comprising a herbicidally effective amount of at least one piperazine compound of the formula I, or an agriculturally useful salt thereof, according to claim 30 and auxiliaries customary for formulating crop protection agents.

43. A method for controlling unwanted vegetation wherein a herbicidally effective amount of at least one piperazine compound of the formula I, or an agriculturally useful salt thereof, according to claim 30 is allowed to act on plants, their seed and/or their habitat.

44. The method of claim 43, wherein the piperazine compound has the general formula Ia: 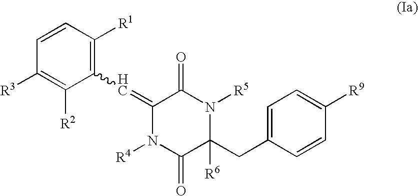
in which
R¹ is cyano or nitro;
R² is hydrogen, fluorine, chlorine, C₁-C₂-alkyl, ethenyl or C₁-C₂-alkoxy;
R³ is fluorine or hydrogen;
R⁴ is methyl;
R⁵ is hydrogen, methyl or ethyl;
R⁶ is methyl or ethyl; and
R⁹ is hydrogen or halogen,
or an agriculturally useful salt of this compound.

45. The method of claim 43, wherein the compound is selected from the group consisting of:
2-[5-benzyl-1,4,5-trimethyl-3,6-dioxopiperazin-2-ylidenemethyl]benzonitrile,
2-[5-benzyl-1,4,5-trimethyl-3,6-dioxopiperazin-2-ylidenemethyl]-3-fluorobenzonitrile,
2-[5-benzyl-1,4,5-trimethyl-3,6-dioxopiperazin-2-ylidenemethyl]-3-methoxybenzonitrile,
2-[5-benzyl-1,4,5-trimethyl-3,6-dioxopiperazin-2-ylidenemethyl]-3,4-difluorobenzonitrile,
2-[5-benzyl-1,4,5-trimethyl-3,6-dioxopiperazin-2-ylidenemethyl]-3-methylbenzonitrile,
2-[5-benzyl-1,4,5-trimethyl-3,6-dioxopiperazin-2-ylidenemethyl]-3-ethenylbenzonitrile,
2-[5-benzyl-1,5-dimethyl-3,6-dioxopiperazin-2-ylidenemethyl]benzonitrile,
2-[5-benzyl-1,5-dimethyl-3,6-dioxopiperazin-2-ylidenemethyl]-3-fluorobenzonitrile,
2-[5-benzyl-1,5-dimethyl-3,6-dioxopiperazin-2-ylidenemethyl]-3-methoxybenzonitrile,
2-[5-benzyl-1,5-dimethyl-3,6-dioxopiperazin-2-ylidenemethyl]-3,4-difluorobenzonitrile,
2-[5-benzyl-1,5-dimethyl-3,6-dioxopiperazin-2-ylidenemethyl]-3-methylbenzonitrile,
2-[5-benzyl-1,5-dimethyl-3,6-dioxopiperazin-2-ylidenemethyl]-3-ethenylbenzonitrile,
2-[5-benzyl-5-ethyl-1,4-dimethyl-3,6-dioxopiperazin-2-ylidenemethyl]benzonitrile,
2-[5-benzyl-5-ethyl-1,4-dimethyl-3,6-dioxopiperazin-2-ylidenemethyl]-3-fluorobenzonitrile,
2-[5-benzyl-5-ethyl-1,4-dimethyl-3,6-dioxopiperazin-2-ylidenemethyl]-3-methoxybenzonitrile,
2-[5-benzyl-5-ethyl-1,4-dimethyl-3,6-dioxopiperazin-2-ylidenemethyl]-3,4-difluorobenzonitrile,
2-[5-benzyl-5-ethyl-1,4-dimethyl-3,6-dioxopiperazin-2-ylidenemethyl]-3-methylbenzonitrile,
2-[5-benzyl-5-ethyl-1,4-dimethyl-3,6-dioxopiperazin-2-ylidenemethyl]-3-ethenylbenzonitrile,
2-[5-benzyl-5-ethyl-1-methyl-3,6-dioxopiperazin-2-ylidenemethyl]benzonitrile,
2-[5-benzyl-5-ethyl-1-methyl-3,6-dioxopiperazin-2-ylidenemethyl]-3-fluorobenzonitrile,
2-[5-benzyl-5-ethyl-1-methyl-3,6-dioxopiperazin-2-ylidenemethyl]-3-methoxybenzonitrile,
2-[5-benzyl-5-ethyl-1-methyl-3,6-dioxopiperazin-2-ylidenemethyl]-3,4-difluorobenzonitrile,
2-[5-benzyl-5-ethyl-1-methyl-3,6-dioxopiperazin-2-ylidenemethyl]-3-methylbenzonitrile,
2-[5-benzyl-5-ethyl-1-methyl-3,6-dioxopiperazin-2-ylidenemethyl]-3-ethenylbenzonitrile,
3-benzyl-6-[1-(2-nitrophenyl)methylidene]-1,3,4-trimethylpiperazine-2,5-dione,
3-benzyl-6-[1-(2-fluoro-6-nitrophenyl)methylidene]-1,3,4-trimethylpiperazine-2,5-dione,
3-benzyl-6-[1-(2,3-difluoro-6-nitrophenyl)methylidene]-1,3,4-trimethylpiperazine-2,5-dione,
3-benzyl-6-[1-(2-methoxy-6-nitrophenyl)methylidene]-1,3,4-trimethylpiperazine-2,5-dione,
3-benzyl-6-[1-(2-methyl-6-nitrophenyl)methylidene]-1,3,4-trimethylpiperazine-2,5-dione,
3-benzyl-6-[1-(2-ethenyl-6-nitrophenyl)methylidene]-1,3,4-trimethylpiperazine-2,5-dione,
3-benzyl-6-[1-(2-nitrophenyl)methylidene]-1,3-dimethylpiperazine-2,5-dione,
3-benzyl-6-[1-(2-fluoro-6-nitrophenyl)methylidene]-1,3-dimethylpiperazine-2,5-dione,
3-benzyl-6-[1-(2,3-difluoro-6-nitrophenyl)methylidene]-1,3-dimethylpiperazine-2,5-dione,
3-benzyl-6-[1-(2-methoxy-6-nitrophenyl)methylidene]-1,3-dimethylpiperazine-2,5-dione,
3-benzyl-6-[1-(2-methyl-6-nitrophenyl)methylidene]-1,3-dimethylpiperazine-2,5-dione,
3-benzyl-6-[1-(2-ethenyl-6-nitrophenyl)methylidene]-1,3-dimethylpiperazine-2,5-dione,
3-benzyl-6-[1-(2-nitrophenyl)methylidene]-3-ethyl-1,4-dimethylpiperazine-2,5-dione,
3-benzyl-6-[1-(2-fluoro-6-nitrophenyl)methylidene]-3-ethyl-1,4-dimethylpiperazine-2,5-dione,
3-benzyl-6-[1-(2,3-difluoro-6-nitrophenyl)methylidene]-3-ethyl-1,4-dimethylpiperazine-2,5-dione,
3-benzyl-6-[1-(2-methoxy-6-nitrophenyl)methylidene]-3-ethyl-1,4-dimethylpiperazine-2,5-dione,
3-benzyl-6-[1-(2-methyl-6-nitrophenyl)methylidene]-3-ethyl-1,4-dimethylpiperazine-2,5-dione,
3-benzyl-6-[1-(2-ethenyl-6-nitrophenyl)methylidene]-3-ethyl-1,4-dimethylpiperazine-2,5-dione,
3-benzyl-6-[1-(2-nitrophenyl)methylidene]-3-ethyl-1-methylpiperazine-2,5-dione,
3-benzyl-6-[1-(2-fluoro-6-nitrophenyl)methylidene]-3-ethyl-1-methylpiperazine-2,5-dione,
3-benzyl-6-[1-(2,3-difluoro-6-nitrophenyl)methylidene]-3-ethyl-1-methylpiperazine-2,5-dione,
3-benzyl-6-[1-(2-methoxy-6-nitrophenyl)methylidene]-3-ethyl-1-methylpiperazine-2,5-dione,
3-benzyl-6-[1-(2-methyl-6-nitrophenyl)methylidene]-3-ethyl-1-methylpiperazine-2,5-dione, and
3-benzyl-6-[1-(2-ethenyl-6-nitrophenyl)methylene]-3-ethyl-1-methylpiperazine-2,5-dione,
2-[5-(4-fluorobenzyl)-1,4,5-trimethyl-3,6-dioxopiperazin-2-ylidenemethyl]-benzonitrile,
2-[5-(4-fluorobenzyl)-1,4,5-trimethyl-3,6-dioxopiperazin-2-ylidenemethyl]-3-fluorobenzonitrile,
2-[5-(4-fluorobenzyl)-1,4,5-trimethyl-3,6-dioxopiperazin-2-ylidenemethyl]-3-methoxybenzonitrile,
2-[5-(4-fluorobenzyl)-1,4,5-trimethyl-3,6-dioxopiperazin-2-ylidenemethyl]-3,4-difluorobenzonitrile,
2-[5-(4-fluorobenzyl)-1,4,5-trimethyl-3,6-dioxopiperazin-2-ylidenemethyl]-3-methylbenzonitrile,
2-[5-(4-fluorobenzyl)-1,4,5-trimethyl-3,6-dioxopiperazin-2-ylidenemethyl]-3-ethenylbenzonitrile,
2-[5-(4-fluorobenzyl)-1,5-dimethyl-3,6-dioxopiperazin-2-ylidenemethyl]-benzonitrile,
2-[5-(4-fluorobenzyl)-1,5-dimethyl-3,6-dioxopiperazin-2-ylidenemethyl]-3-fluorobenzonitrile,
2-[5-(4-fluorobenzyl)-1,5-dimethyl-3,6-dioxopiperazin-2-ylidenemethyl]-3-methoxybenzonitrile,
2-[5-(4-fluorobenzyl)-1,5-dimethyl-3,6-dioxopiperazin-2-ylidenemethyl]-3,4-difluorobenzonitrile,
2-[5-(4-fluorobenzyl)-1,5-dimethyl-3,6-dioxopiperazin-2-ylidenemethyl]-3-methylbenzonitrile,
2-[5-(4-fluorobenzyl)-1,5-dimethyl-3,6-dioxopiperazin-2-ylidenemethyl]-3-ethenylbenzonitrile,
2-[5-(4-fluorobenzyl)-5-ethyl-1,4-dimethyl-3,6-dioxopiperazin-2-ylidenemethyl]-benzonitrile,
2-[5-(4-fluorobenzyl)-5-ethyl-1,4-dimethyl-3,6-dioxopiperazin-2-ylidenemethyl]-3-fluorobenzonitrile,
2-[5-(4-fluorobenzyl)-5-ethyl-1,4-dimethyl-3,6-dioxopiperazin-2-ylidenemethyl]-3-methoxybenzonitrile,
2-[5-(4-fluorobenzyl)-5-ethyl-1,4-dimethyl-3,6-dioxopiperazin-2-ylidenemethyl]-3,4-difluorobenzonitrile,
2-[5-(4-fluorobenzyl)-5-ethyl-1,4-dimethyl-3,6-dioxopiperazin-2-ylidenemethyl]-3-methylbenzonitrile,
2-[5-(4-fluorobenzyl)-5-ethyl-1,4-dimethyl-3,6-dioxopiperazin-2-ylidenemethyl]-3-ethenylbenzonitrile,
2-[5-(4-fluorobenzyl)-5-ethyl-1-methyl-3,6-dioxopiperazin-2-ylidenemethyl]-benzonitrile,
2-[5-(4-fluorobenzyl)-5-ethyl-1-methyl-3,6-dioxopiperazin-2-ylidenemethyl]-3-fluorobenzonitrile,
2-[5-(4-fluorobenzyl)-5-ethyl-1-methyl-3,6-dioxopiperazin-2-ylidenemethyl]-3-methoxybenzonitrile,
2-[5-(4-fluorobenzyl)-5-ethyl-1-methyl-3,6-dioxopiperazin-2-ylidenemethyl]-3,4-difluorobenzonitrile,
2-[5-(4-fluorobenzyl)-5-ethyl-1-methyl-3,6-dioxopiperazin-2-ylidenemethyl]-3-methylbenzonitrile,
2-[5-(4-fluorobenzyl)-5-ethyl-1-methyl-3,6-dioxopiperazin-2-ylidenemethyl]-3-ethenylbenzonitrile,
3-(4-fluorobenzyl)-6-[1-(2-nitrophenyl)methylidene]-1,3,4-trimethylpiperazine-2,5-dione,
3-(4-fluorobenzyl)-6-[1-(2-fluoro-6-nitrophenyl)methylidene]-1,3,4-trimethylpiperazine-2,5-dione,
3-(4-fluorobenzyl)-6-[1-(2,3-difluoro-6-nitrophenyl)methylidene]-1,3,4-trimethylpiperazine-2,5-dione,
3-(4-fluorobenzyl)-6-[1-(2-methoxy-6-nitrophenyl)methylidene]-1,3,4-trimethylpiperazine-2,5-dione,
3-(4-fluorobenzyl)-6-[1-(2-methyl-6-nitrophenyl)methylidene]-1,3,4-trimethylpiperazine-2,5-dione,
3-(4-fluorobenzyl)-6-[1-(2-ethenyl-6-nitrophenyl)methylidene]-1,3,4-trimethylpiperazine-2,5-dione,
3-(4-fluorobenzyl)-6-[1-(2-nitrophenyl)methylidene]-1,3-dimethylpiperazine-2,5-dione,
3-(4-fluorobenzyl)-6-[1-(2-fluoro-6-nitrophenyl)methylidene]-1,3-dimethylpiperazine-2,5-dione,
3-(4-fluorobenzyl)-6-[1-(2,3-difluoro-6-nitrophenyl)methylidene]-1,3-dimethylpiperazine-2,5-dione,
3-(4-fluorobenzyl)-6-[1-(2-methoxy-6-nitrophenyl)methylidene]-1,3-dimethylpiperazine-2,5-dione,
3-(4-fluorobenzyl)-6-[1-(2-methyl-6-nitrophenyl)methylidene]-1,3-dimethylpiperazine-2,5-dione,
3-(4-fluorobenzyl)-6-[1-(2-ethenyl-6-nitrophenyl)methylidene]-1,3-dimethylpiperazine-2,5-dione,
3-(4-fluorobenzyl)-6-[1-(2-nitrophenyl)methylidene]-3-ethyl-1,4-dimethylpiperazine-2,5-dione,
3-(4-fluorobenzyl)-6-[1-(2-fluoro-6-nitrophenyl)methylidene]-3-ethyl-1,4-dimethylpiperazine-2,5-dione,
3-(4-fluorobenzyl)-6-[1-(2,3-difluoro-6-nitrophenyl)methylidene]-3-ethyl-1,4-dimethylpiperazine-2,5-dione,
3-(4-fluorobenzyl)-6-[1-(2-methoxy-6-nitrophenyl)methylidene]-3-ethyl-1,4-dimethylpiperazine-2,5-dione,
3-(4-fluorobenzyl)-6-[1-(2-methyl-6-nitrophenyl)methylidene]-3-ethyl-1,4-dimethylpiperazine-2,5-dione,
3-(4-fluorobenzyl)-6-[1-(2-ethenyl-6-nitrophenyl)methylidene]-3-ethyl-1,4-dimethylpiperazine-2,5-dione,
3-(4-fluorobenzyl)-6-[1-(2-nitrophenyl)methylidene]-3-ethyl-1-methylpiperazine-2,5-dione,
3-(4-fluorobenzyl)-6-[1-(2-fluoro-6-nitrophenyl)methylidene]-3-ethyl-1-methylpiperazine-2,5-dione,
3-(4-fluorobenzyl)-6-[1-(2,3-difluoro-6-nitrophenyl)methylidene]-3-ethyl-1-methylpiperazine-2,5-dione,
3-(4-fluorobenzyl)-6-[1-(2-methoxy-6-nitrophenyl)methylidene]-3-ethyl-1-methylpiperazine-2,5-dione,
3-(4-fluorobenzyl)-6-[1-(2-methyl-6-nitrophenyl)methylidene]-3-ethyl-1-methylpiperazine-2,5-dione,
3-(4-fluorobenzyl)-6-[1-(2-ethenyl-6-nitrophenyl)methylidene]-3-ethyl-1-methylpiperazine-2,5-dione,
2-[5-benzyl-1,4,5-trimethyl-3,6-dioxopiperazin-2-ylidenemethyl]-3-bromobenzonitrile,
2-[5-benzyl-1,4,5-trimethyl-3,6-dioxopiperazin-2-ylidenemethyl]isophthalonitrile,
3-benzyl-6-[1-(2,3-difluoro-6-nitrophenyl)methylidene]-1,3,4-trimethylpiperazine-2,5-dione,
3-benzyl-6-[1-(2-nitro-5-methoxyphenyl)methylidene]-1,3,4-trimethylpiperazine-2,5-dione,
2-[5-benzyl-1,4,5-trimethyl-3,6-dioxopiperazin-2-ylidenemethyl]-3-nitrobenzonitrile,
3-benzyl-6-[1-(2-ethenyl-6-nitrophenyl)methylidene]-1,3,4-trimethylpiperazine-2,5-dione,
3-benzyl-6-[1-(3-chloro-2-nitrophenyl)methylidene]-1,3,4-trimethylpiperazine-2,5-dione,
3-benzyl-6-[1-(2-nitro-6-trifluoromethylphenyl)methylidene]-1,3,4-trimethylpiperazine-2,5-dione,
2-[5-benzyl-1,5-dimethyl-3,6-dioxopiperazin-2-ylidenemethyl]benzonitrile,
3-benzyl-6-[1-(2-methoxy-6-nitrophenyl)methylidene]-1,3,4-trimethylpiperazine-2,5-dione,
2-[5-benzyl-1,4,5-trimethyl-3,6-dioxopiperazin-2-ylidenemethyl]-4-fluorobenzonitrile,
2-[5-benzyl-1,4,5-trimethyl-3,6-dioxopiperazin-2-ylidenemethyl]-5-methylbenzonitrile,
2-[5-benzyl-1,4,5-trimethyl-3,6-dioxopiperazin-2-ylidenemethyl]-6-fluorobenzonitrile,
3-benzyl-1,3,4-trimethyl-6-[2-(1-methyl-1H-pyrrol-2-yl)benzylidene]piperazine-2,5-dione,
3-benzyl-6-(2-furan-2-yl-benzylidene)-1,3,4-trimethylpiperazine-2,5-dione,
2-[5-benzyl-5-fluoromethyl-1,4-dimethyl-3,6-dioxopiperazin-2-ylidenemethyl]-benzonitrile,
3-benzyl-1,3,4-trimethyl-6-(4-methyl-2-nitrobenzylidene)piperazine-2,5-dione,
2-[5-benzyl-1,4,5-trimethyl-3,6-dioxopiperazin-2-ylidenemethyl]-4-methoxybenzonitrile,
3-benzyl-6-[2-(2-chloropyrimidin-5-yl)benzylidene]-1,3,4-trimethylpiperazine-2,5-dione,
3-benzyl-6-[2-(6-fluoropyridin-2-yl)benzylidene]-1,3,4-trimethylpiperazine-2,5-dione,
2-[5-benzyl-1,4,5-trimethyl-3,6-dioxopiperazin-2-ylidenemethyl]-4-fluorobenzonitrile,
2-[5-benzyl-1,4,5-trimethyl-3,6-dioxopiperazin-2-ylidenemethyl]-4-trifluoromethylbenzonitrile,
3-benzyl-1,3,4-trimethyl-6-[2-(1-methyl-1H-imidazol-2-yl)benzylidene]piperazine-2,5-dione,
3-benzyl-3-fluoromethyl-1,4-dimethyl-6-(2-nitrobenzylidene)piperazine-2,5-dione,
3-benzyl-6-(5-bromo-2-nitrobenzylidene)-1,3,4-trimethylpiperazine-2,5-dione,
2-[5-benzyl-1,4,5-trimethyl-3,6-dioxopiperazin-2-ylidenemethyl]-4-difluoromethoxybenzonitrile,
2-[5-benzyl-1,4,5-trimethyl-3,6-dioxopiperazin-2-ylidenemethyl]-4-methanesulfonylbenzonitrile,
2-[5-benzyl-1,4,5-trimethyl-3,6-dioxopiperazin-2-ylidenemethyl]-4-methanesulfinylbenzonitrile,
2-[5-benzyl-1,4,5-trimethyl-3,6-dioxopiperazin-2-ylidenemethyl]-4-methylsulfanylbenzonitrile,
2-[5-benzyl-1,4,5-trimethyl-3,6-dioxopiperazin-2-ylidenemethyl]-3-fluoro-4-methoxybenzonitrile,
2-[5-benzyl-1,4,5-trimethyl-3,6-dioxopiperazin-2-ylidenemethyl]-4,6-difluorobenzonitrile,
3-benzyl-1,3,4-trimethyl-6-[2-(2-methyl-2H-pyrazol-3-yl)benzylidene]piperazine-2,5-dione,
3-benzyl-1,3,4-trimethyl-6-[2-(5-methylthiophen-2-yl)benzylidene]piperazine-2,5-dione,
3-benzyl-1,3,4-trimethyl-6-[2-(3-methylthiophen-2-yl)benzylidene]piperazine-2,5-dione,
2-[5-benzyl-4-ethyl-1,5-dimethyl-3,6-dioxopiperazin-2-ylidenemethyl]benzonitrile,
2-[5-benzyl-4-isopropyl-1,5-dimethyl-3,6-dioxopiperazin-2-ylidenemethyl]-benzonitrile,
2-[5-benzyl-4-butyl-1,5-dimethyl-3,6-dioxopiperazin-2-ylidenemethyl]benzonitrile,
2-[4-allyl-5-benzyl-1,5-dimethyl-3,6-dioxopiperazin-2-ylidenemethyl]benzonitrile,
2-[5-benzyl-5-trifluoromethyl-1,4-dimethyl-3,6-dioxopiperazin-2-ylidenemethyl]-benzonitrile,
3-benzyl-6-[1-(2-nitrophenyl)methylidene]-1,4-dimethyl-3-trifluoromethylpiperazine-2,5-dione,
3-benzyl-6-[2-(6-chloropyridin-3-yl)benzylidene]-1,3,4-trimethylpiperazine-2,5-dione,
2-[5-benzyl-1,5-dimethyl-4-prop-2-ynyl-3,6-dioxopiperazin-2-ylidenemethyl]-benzonitrile,
3-(3-fluorobenzyl)-6-[1-(2-nitrophenyl)methylidene]-1,3,4-trimethylpiperazine-2,5-dione,
3-(3,5-difluorobenzyl)-6-[1-(2-nitrophenyl)methylidene]-1,3,4-trimethylpiperazine-2,5-dione,
2-[5-(2,3-difluorobenzyl)-1,4,5-trimethyl-3,6-dioxopiperazin-2-ylidenemethyl]-benzonitrile,
2-[5-(2,5-difluorobenzyl)-1,4,5-trimethyl-3,6-dioxopiperazin-2-ylidenemethyl]-benzonitrile,
2-[5-(2,6-difluorobenzyl)-1,4,5-trimethyl-3,6-dioxopiperazin-2-ylidenemethyl]-benzonitrile,
2-[5-(2-difluoromethoxybenzyl)-1,4,5-trimethyl-3,6-dioxopiperazin-2-ylidenemethyl]benzonitrile,
2-[5-(3-difluoromethoxybenzyl)-1,4,5-trimethyl-3,6-dioxopiperazin-2-ylidenemethyl]benzonitrile,
2-[5-(3-trifluoromethylbenzyl)-1,4,5-trimethyl-3,6-dioxopiperazin-2-ylidenemethyl]benzonitrile,
3-(3-fluorobenzyl)-6-[1-(2-nitrophenyl)methylidene]-1,3,4-trimethylpiperazine-2,5-dione,
2-[5-(2-cyanobenzyl)-1,4,5-trimethyl-3,6-dioxopiperazin-2-ylidenemethyl]-benzonitrile,
2-[5-(3-cyanobenzyl)-1,4,5-trimethyl-3,6-dioxopiperazin-2-ylidenemethyl]-benzonitrile,
2-[5-(3,5-difluorobenzyl)-1,4,5-trimethyl-3,6-dioxopiperazin-2-ylidenemethyl]-benzonitrile,
2-[5-(3-nitrobenzyl)-1,4,5-trimethyl-3,6-dioxopiperazin-2-ylidenemethyl]-benzonitrile,
2-[5-(4-fluoro-3-methylbenzyl)-1,4,5-trimethyl-3,6-dioxopiperazin-2-ylidenemethyl]benzonitrile,
2-[5-(4-fluoro-3-methoxybenzyl)-1,4,5-trimethyl-3,6-dioxopiperazine-2-ylidenemethyl]benzonitrile,
1-allyl-3-benzyl-3,4-dimethyl-6-[1-(2-nitrophenyl)methylidene]piperazine-2,5-dione and
3-benzyl-6-[1-(2-nitrophenyl)methylidene]-1-prop-2-ynyl-3,4-dimethylpiperazine-2,5-dione.

46. The method of claim 43, wherein the exo double bond at the piperazine ring has the (Z) configuration.

47. The method of claim 43, wherein R¹ is cyano, nitro or a 5- or 6-membered heteroaromatic radical which has either 1, 2 or 3 nitrogen atoms or 1 oxygen or 1 sulfur atom and, if appropriate, 1 or 2 nitrogen atoms as ring members and which is unsubstituted or may have 1 or 2 substituents selected from R^{ia}.

48. The method of claim 43, wherein R¹ is halogen, in particular chlorine or bromine.

49. The method of claim 43, in which R⁴ is methyl.
